(19) United States
(12) Patent Application Publication
(10) Pub. No.: US 2015/0105278 A1
(43) Pub. Date: Apr. 16, 2015

(54) METHODS OR RISK ASSESSMENT OF PML AND RELATED APPARATUS

(71) Applicant: WESTFAELISCHE WILHELMS-UNIVERSITAET MUENSTER, Muenster (DE)

(72) Inventors: Nicholas Schwab, Muenster (DE); Tilman Schneider-Hohendorf, Muenster (DE); Heinz Wiendl, Muenster (DE)

(73) Assignee: WESTFAELISCHE WILHELMS-UNIVERSITAET MUENSTER, Muenster (DE)

(21) Appl. No.: 14/352,402

(22) PCT Filed: Oct. 16, 2012
(86) PCT No.: PCT/EP2012/070465

§ 371 Date: Apr. 17, 2014

(30) Foreign Application Priority Data
Oct. 17, 2011 (EP) 11185439.4
Mar. 7, 2012 (EP) 12158369.4

(51) Int. Cl. G01N 33/68; C12Q 1/68

(52) U.S. Cl. CPC G01N 33/6872; G01N 33/6893; C12Q 1/6883; G01N 2333/70564; G01N 2333/70553; G01N 2800/28; G01N 2800/50; G01N 2800/52; C12Q 2600/158; C12Q 2600/118
USPC 506/9; 435/29; 435/7.24; 435/6.11; 435/6.12; 506/7; 530/387.3

(57) ABSTRACT

The invention provides a method of assessing the risk of occurrence of progressive multifocal leukoencephalopathy (PML) in a subject as well as a method of stratifying a subject undergoing VLA-4 blocking agent treatment for suspension of VLA-4 blocking agent treatment. These methods comprise detecting the level of L-selectin (CD62L) and optionally LFA-1 expressing T cells in a sample from the subject.

### CROSS-REFERENCE TO RELATED APPLICATIONS

The present application claims the benefit of and the priority to an application for “Risk Stratification of Patients Receiving VLA-4 Blocking Agents” filed on 17 Oct. 2011 with the European Patent Office, and there duly assigned serial number EP 11 185 439. The present application further claims the benefit of and the priority to an application for “Methods of Risk Assessment of PML” filed on 7 Mar. 2012 with the European Patent Office, and there duly assigned serial number EP 12 158 369. The contents of said applications filed on 17 Oct. 2011 and 7 Mar. 2012 are incorporated herein by reference for all purposes, including an incorporation of any element or part of the description, claims or drawings not contained herein and referred to in Rule 20.5(a) of the PCT, pursuant to Rule 4.18 of the PCT.

### FIELD OF THE INVENTION

The present invention generally relates to evaluating the immune response of a subject to a therapeutic agent. The present invention also relates to a method of treating a subject with a demyelinating disease or an autoimmune disease. In another aspect, the present invention concerns the identification of patients at lower or higher risk for developing progressive multifocal leukoencephalopathy. More specifically, the present invention relates to a method of assessing the risk of occurrence of progressive multifocal leukoencephalopathy (PML) in a subject. The invention also relates to a method of stratifying a subject undergoing VLA-4 blocking agent treatment for suspension of this VLA-4 blocking agent treatment. Provided is further a method of administering a VLA-4 blocking agent to a subject so as to avoid the additional occurrence of PML.

### BACKGROUND OF THE INVENTION

Multifocal Progressive multifocal leukoencephalopathy (PML) is a severe, rapidly progressive disease that destroys the myelin coating which protects nerve cells. It is caused by the JC virus, a common polymavirus. Through poorly understood interactions between host and viral factors, JCV undergoes alterations in the regulatory region and mutations in the coat protein VP1 to cause lytic infection of myelin-producing oligodendrocytes, leading to development of progressive multifocal leukoencephalopathy. PML occurs generally in severely immunocompromised individuals and in patients receiving certain immunosuppressive therapiestherapy of multiple sclerosis and Crohn's disease.

Multiple sclerosis (MS) is a chronic, inflammatory central nervous system (CNS) disease characterized pathologically by demyelination. MS has also been classified as an autoimmune disease. MS disease activity can be monitored by cranial scans, including magnetic resonance imaging (MRI) of the brain, accumulation of disability, as well as rate and severity of relapses. There are five distinct disease stages and/or types of MS, namely, (1) benign multiple sclerosis; (2) relapsing-remitting multiple sclerosis (RRMS); (3) secondary progressive multiple sclerosis (SPMS); (4) progressive relapsing multiple sclerosis (PRMS); and (5) primary progressive multiple sclerosis (PPMS).

The VLA-4 blocking agent, Natalizumab, was first approved in 2004 by the U.S. Food and Drug Administration (FDA) for the treatment of multiple sclerosis. It was subsequently withdrawn from the market after it was linked with three cases of PML. After a review of safety information and no further deaths, the drug was returned to the US market in 2006 under a special prescription program.

So far there have been over 150 cases of JCV-induced progressive multifocal leukencephalopathy (PML) associated with the treatment of MS patients with Natalizumab. It is still largely unknown how the treatment with blocking VLA-4 interferes with JC virus control or immune surveillance. Crohn's disease is a type of inflammatory bowel disease. It typically manifests in the gastrointestinal tract and can be categorized by the specific tract region affected. It is thought to be an autoimmune disease, in which the body's immune system attacks the gastrointestinal tract, causing inflammation of gastrointestinal tract. The disease manifestations usually are isolated to the digestive tract, but other manifestations such as inflammation of skin structures, the eyes, and the joints have been well described. The disease is known to have spontaneous exacerbations and remissions. Unfortunately, the cause of Crohn's disease is not known, and there is no known cure for Crohn's disease.

Crohn's disease has an immune response pattern that includes an increased production of interleukin-12, tumour necrosis factor (TNF) and interferon-y. Tumor necrosis factor (TNF) has been identified as an important cytokine in the pathogenesis of Crohn's disease, with elevated concentrations playing a role in pathologic inflammation. The increased production of TNF by macrophages in patients with Crohn's disease results in elevated concentrations of TNF in the stool, blood, and mucosa. In recent years, biologic response modifiers that inhibit TNF activity have become potential therapies for treating Crohn's disease.

The humanized monoclonal immunoglobulin Natalizumab is used in the treatment of both MS and Crohn's disease. Natalizumab is both clinically effective and generally well-tolerated. However, Natalizumab treatment for longer than 18 months has been found to be associated with an enhanced risk of developing PML. PML has almost exclusively been found in immunocompromised individuals, especially in subjects with reduced cellular immunity. It has also been reported in rheumatic diseases. PML has for example been found in individuals with hematological malignancies and lymphoproliferative diseases, individuals with Hodgkin's lymphoma, individuals with systemic lupus erythematosus or subjects receiving immunosuppressive medication such as transplant patients. In addition to Natalizumab therapy, PML has also been found to be associated with therapy using the monoclonal antibodies Rituximab, used in the treatment of lymphomas, leukemias, transplant rejection and certain autoimmune disorders, and Efalizumab, formerly used in the treatment of autoimmune diseases, in particular psoriasis. In view of the risk of PML, Efalizumab has currently been withdrawn from the U.S. market. Natalizumab was now restricted as a monotherapy for relapsing remitting multiple sclerosis (RRMS) patients with high disease activity.

So far there have been over 150 cases of JCV-induced PML associated with the treatment of MS patients with Natalizumab, where the mortality rate of so far 20%. It is still largely unknown how the treatment with blocking integrin α4β1/VLA-4 interferes with JCV control or immune surveillance (Tan, C. S, and Koralnik, I. J., Lancet Neurol. (2010) 9, 4, 425-437).

Prognosis of PML is poor, since no specific therapy is available. While only 20% of the Natalizumab-treated PML patients so far died, the overall mortality of PML has been reported to be above 50%. In the absence of any therapy it would be particularly helpful to be able to predict the risk whether an individual is likely to develop PML. Hence, there exists a need for means to determine at an early stage, i.e. before the onset of the disease, whether an HIV positive individual is likely to suffer from PML.

Recent studies suggest that patients under treatment with the very late activation antigen 4 (VLA-4, integrin α4β1) blocking agent Natalizumab for more than 12 months are at elevated risk for PML, with the risk increasing after approximately 18 months of treatment, and can reach risk levels of up to 1:120. It is not known if the risk of developing PML continues to increase, remains the same, or decreases after a patient has been on Natalizumab for more than three years. Since there is a clear risk association between Natalizumab and the development of PML after long-term treatment of the VLA-4 blocking agent Natalizumab, there is an urgent need to identify those patients who are more prone to PML. However, only few candidates have evolved: (1) treatment duration, (2) pre-treatment with immunosuppressive drugs, and (3) presence of JCV antibodies in serum.

U.S. Pat. Pub. No. 2010/0196318 discloses testing for serum anti-JCV antibody prior to initiating Natalizumab therapy in patients. However, the detection of JCV antibody in an individual does not predict the risk for PML and therefore cannot advise a medical professional whether or not to continue the treatment. U.S. Pat. Pub. No. 2009/0216107 discloses a method of screening patients undergoing Natalizumab treatment by testing the patient's cerebrospinal fluid to detect the presence of cytomegalovirus, JCV, Toxoplasma gondii, Epstein-Barr virus, Cryptococcus neoformans and tuberculosis by PCR, as well as examining the retinal status to detect the presence of ocular cytomegalovirus. If an indication of the presence of the virus is detected, Natalizumab treatment should be discontinued. However, such methods are only precautionary measures which also do not indicate a risk of developing PML. There still remains a need to develop a method to determine the susceptibility to PML for patients who receive VLA-4 blocking agent on an individual basis. It would be advantageous if the determination could help the practitioner to identify patients who are particularly prone to PML or stop the treatment in time before the immune competence of the subject deteriorates.

It is therefore an object of the present invention to provide a method and apparatus that is suitable for determining the risk for PML development in a subject. It would be advantageous if such method and apparatus can be used to monitor the immune competence of patients receiving or expected to receive Natalizumab thus to avoid the possible development of PML or even another complication at a later stage. It is a further object of the invention to provide a method for assessing the likelihood of PML occurrence in a subject suffering from HIV. These objects are solved by the method of claim 1. Also provided herein are apparatuses such as kits and assays related to these purposes.

It is also another objective of the present invention to develop an improved method of treating patients with VLA-4 blocking agents.

### SUMMARY OF THE INVENTION

The present invention generally relates to the determination of a subject's immune competence. More specifically, the present invention provides inter alia a method for assessing the susceptibility to a condition associated with JC virus and a method of PML risk stratification, and means for the methods. In one aspect, this disclosure provides a method for determining the susceptibility of a patient undergoing VLA-4 blocking agent treatment to a JCV-induced disease.

The present invention is based, at least in part, on the finding that the binding of VLA-4 influences the expression of adhesion molecules CD62L and LFA-1 and basic immune cell functions such as migratory capacity. Without being limited to any particular theory, it has been discovered that some adhesion molecules, CD62L and LFA-1 included, are differentially expressed in patients who developed PML.

The methods and uses provided by the present invention are based on employing L-selectin (CD62L) and optionally lymphocyte function-associated antigen-1 (LFA-1) as a biomarker for identifying a predisposition of a subject of developing PML. Use of such molecules as biomarkers, in conjunction with further biomarkers or tests, provides indication of which patients are more likely to suffer from PML. The biomarkers provided in the present invention can assist physicians in the determination of appropriate therapeutic regimen. In the context of the present invention CD62L levels may be determined using any desired technique. In some embodiments means may be employed that indirectly indicate CD62L levels and optionally LFA-1 levels, for example by assessing indicators from which levels of CD62L and optionally LFA-1 can be inferred.

Accordingly, the biomarkers provided in the present invention may be advantageously used to assess the immune competence of subjects, preferably subjects who are receiving or expected to receive long-term VLA-4 blocking agent treatment, as well as to determine the risk of the subject to suffer from PML.

According to a first aspect, the present invention provides a method of assessing the risk of occurrence of PML in a subject. The method can be seen as method of treating patients with a demyelinating disease or an autoimmune disease, including, but not limited to, multiple sclerosis (MS), Crohn's disease (CD) and rheumatoid arthritis. As mentioned earlier, a number of MS and CD patients receiving VLA-4 blocking agents may become susceptible to PML. Therefore, in another aspect the present invention provides a method of treatment comprising administering VLA-4 blocking agents, measuring the expression of CD62L and optionally LFA-1 on T cells in sample from a patient to be treated, and stopping or continuing the administration of VLA-4 blocking agents based on the measured level of the expression.

In another aspect, the present invention provides a way of screening such patients who are more prone to PML. The method generally includes providing a sample from the subject and detecting the level of CD62L expressing T cells in the sample from the subject.

In another aspect, the present invention provides a method of predicting whether a patient is at risk of developing PML. The method comprises a) measuring the expression of at least one biomarker from T cell, and b) comparing the expression with a reference value. The term “predict” generally means to determine in advance. As used herein, the term “predict” means to determine the risk for PML or assess the risk or occurrence for PML if the patient continues to receive VLA-4 blocking agent treatment. The present invention is also related to a kit comprising CD62L binding assay and optionally LFA-1 binding assay and use thereof for determining the susceptibility of a patient to progressive multifocal leukoencephalopathy. According to a particular embodiment of the present invention, the T cells are CD3⁺ T cells. In one embodiment the method includes detecting the level of CD62L expressing T cells in a sample from the subject. According to a particular embodiment the method according to the first aspect further includes detecting the level of LFA-1 expressing T cells in the sample from the subject. According to some embodiments of the method according to the first aspect, the expression of CD62L is monitored at certain, e.g. predetermined, time intervals. According to a particular embodiment of the method according to the first aspect, the expression of both CD62L and LFA-1 is monitored at certain, e.g. predetermined, time intervals.

According to a further embodiment of the method according to the first aspect, the subject is diagnosed as being in need of treatment with a VLA-4 blocking agent. In such an embodiment the level of CD62L expressing T cells and optionally LFA-1 expressing T cells in the sample from the subject may be analysed.

In one embodiment of the method according to the first aspect a decreased level of CD62L expressing T cells and optionally of LFA-1 expressing T cells, relative to a threshold value, indicates a risk of occurrence of PML. A level of CD62L expressing T cells and optionally of LFA-1 expressing T cells that is about at a threshold value or above a threshold value indicates no risk of occurrence of PML when compared to healthy subjects.

According to an embodiment of the method according to the first, the second, the third and/or the fourth aspect, the method includes comparing the level of CD62L and optionally LFA-1 expressing T cells in the sample to a threshold value.

According to further embodiments of the method according to the first, the second, the third and/or the fourth aspect, the method includes determining the migration of CD45⁻ CD49d⁺ immune cells, such as CD45⁺CD49d⁺ T cells. In some embodiments migration is measured using a transendothelial chemotaxis assay. In some embodiments migration is measured using a chemotaxis assay, for instance employing a blank permeable membrane.

In a related second aspect the invention provides a method of screening one or more individuals for susceptibility to a condition associated with JCV infection. The method generally includes providing a sample from each of the one or more subjects. The method includes detecting the level of CD62L expressing T cells and/or detecting the level of LFA-1 expressing T cells in the sample from each of the one or more subjects.

In some embodiments of the method according to the second aspect an altered, such as a decreased or an increased, level of CD62L and/or LFA-1 expressing T cells, relative to a threshold value, may indicate a susceptibility to a condition associated with JCV infection. In such embodiments a method according to the second aspect may include determining that the subject is susceptible to a condition associated with JCV infection.

In one embodiment of the method according to the second aspect, a decreased level of CD62L expressing T cells and/or of LFA-1 expressing T cells, relative to a threshold value, indicates that the subject is susceptible to a condition associated with JCV infection. A level of CD62L expressing T cells and optionally of LFA-1 expressing T cells that is about at a threshold value or above a threshold value indicates that the subject is not susceptible to a condition associated with JCV infection when compared to healthy subjects.

According to a particular embodiment, the method according to the second aspect includes comparing the level of CD62L and optionally LFA-1 expressing T cells in the sample to a threshold value.

In a further aspect there are provided methods of monitoring the susceptibility of a JCV related condition in a subject. The methods include monitoring the level of CD62L1 expressing T cells of the subject. Generally these T cells are included, including provided, in a sample from the subject. In some embodiments the method further includes monitoring the level of LFA-1 expressing T cells of the subject. Monitoring the expression of CD62L and optionally LFA-1 on T cells is generally carried out using a sample from the subject. Monitoring may be carried out at predetermined time intervals. In some embodiments monitoring begins prior to a treatment. In some embodiments a respective treatment may be a VLA-4 blocking agent treatment.

In a related aspect there is provided a method of monitoring the susceptibility of a subject to PML. The method includes monitoring the level of expression of CD62L on T cells. In some embodiments the method further includes monitoring the level of expression of LFA-1 on T cells. Generally these T cells are included, including provided, in a sample from the subject. In a further aspect there is disclosed a method of screening patients who are known or suspected to be prone to occurrence of PML. The method generally includes detecting the level of CD62L expressing T cells in a sample from the subject. In some embodiments the method further includes detecting the level of LFA-1 expressing T cells in the sample. The method may also include comparing the result to a threshold value.

According to a third aspect, the invention provides a method of stratifying a subject that/who is undergoing VLA-4 blocking agent treatment for suspension of VLA-4 blocking agent treatment. The method generally includes providing a sample from the subject. The method further includes detecting the level of T cells in the sample from the subject, with the T cells expressing CD62L. In some embodiments of the method according to the third aspect, the T cells are expressing both CD62L and LFA-1.

In some embodiments of the method, an altered, such as a decreased or an increased, level of CD62L and optionally LFA-1 expressing T cells, relative to a threshold value, may indicate a risk of occurrence of PML. In such embodiments a method according to the third aspect may include stratifying the subject for suspension of VLA-4 blocking agent treatment.

In one embodiment of the method, a decreased level of CD62L expressing T cells and optionally of LFA-1 expressing T cells expressing T cells, relative to a threshold value, indicates a risk of occurrence of PML. A level of CD62L expressing T cells and optionally of LFA-1 expressing T cells that is about at a threshold value or above a threshold value indicates no risk of occurrence of PML when compared to healthy subjects.

According to a particular embodiment, the method includes comparing the level of CD62L and optionally LFA-1 expressing T cells in the sample to a threshold value.

According to a fourth aspect, the invention provides a method of stratifying a subject in need of VLA-4 blocking agent treatment for risk of PML occurrence. The method generally includes providing a sample from the subject. The method further includes detecting the level of T cells which express CD62L, in the sample from the subject.

In some embodiments of the method according to the fourth aspect an altered, such as a decreased or an increased, level of CD62L expressing T cells, relative to a threshold value, may indicate an increased risk of PML occurrence. In such embodiments a method according to the fourth aspect may include stratifying the subject for risk of PML occurrence. According to a particular embodiment, the method according to the fourth aspect includes comparing the level of CD62L expressing T cells in the sample to a threshold value.

According to a further aspect, the invention relates to the in-vitro use of a capture probe, which is specific for CD62L, for assessing the risk of occurrence of PML in a subject.

In typical embodiments the capture probe is an immunoglobulin or a proteinaceous binding molecule with immunoglobulin-like functions, with the binding molecule or the immunoglobulin being specific for CD62L.

The subject may in some embodiments suffer from an autoimmune disease, such as HIV, The subject may be undergoing treatment with one or more VLA-4 blocking agents.

According to a further aspect the invention relates to the in-vitro use of a capture probe, which is specific for LFA-1, for assessing the risk of occurrence of PML in a subject. The subject may in some embodiments suffer from an autoimmune disease. In some embodiments the subject may undergo treatment with one or more VLA-4 blocking agents. In typical embodiments the capture probe is an immunoglobulin or a proteinaceous binding molecule with immunoglobulin-like functions specific for LFA-1.

According to another aspect, the invention relates to the in-vitro use of a capture probe, which is specific for at least one of CD11A, CD18 and LFA-1, for assessing the risk of occurrence of PML in a subject.

In typical embodiments the capture probe is an immunoglobulin or a proteinaceous binding molecule with immunoglobulin-like functions specific for at least one of CD11A, CD18 and LFA-1.

According to a further aspect, the invention relates to the in-vitro use of a capture probe, which is specific for at least one of CD11A, CD18 and LFA-1, for stratifying a subject undergoing VLA-4 blocking agent treatment for suspension of the VLA-4 blocking agent treatment.

In typical embodiments the capture probe is an immunoglobulin or a proteinaceous binding molecule with immunoglobulin-like functions specific for at least one of CD11A, CD18 and LFA-1.

According to another aspect the invention relates to the use of a CD62L and optionally LFA-1 binding assay kit for determining the susceptibility of a subject undergoing or intended to undergo VLA-4 blocking agent treatment to PML.

According to a particular embodiment of the use according to this aspect the CD62L and optionally LFA-1 binding assay kit employs a CD62L capture probe and optionally a LFA-1 capture probe. According to a further particular embodiment of the use, the subject is a multiple sclerosis or a Crohn's disease patient.

According to a further aspect the invention relates to the use of a CD62L binding assay kit for determining the susceptibility of an HIV positive subject to PML. The CD62L binding assay kit comprises a CD62L capture probe.

According to another aspect there is provided a method of treating a subject. The method includes administering one or more VLA-4 blocking agents to the subject. The method may further include detecting the level of expression of one or more biomarkers disclosed herein on T cells, such as CD3⁺ T cells, of the subject. In some embodiments the expression of the one or more biomarkers on T cells is monitored. Generally these T cells are included, including provided, in a sample from the subject. The method may also include determining migration of immune cells, such as CD45 CD49⁺ cells and T cells. In some embodiments, a respective biomarker is one or more of CD62L and LFA-1. In some embodiments the method may include both (i) detecting the level of expression of the one or more biomarkers on T cells and (ii) determining migration of immune cells. In some embodiments the subject may be suffering from a demyelinating or autoimmune disorder.

The subject is in some embodiments suffering from a pathologic inflammatory disease within the CNS. The subject may in some embodiments be diagnosed to have an autoimmune disease, such as multiple sclerosis, e.g. relapsing remitting multiple sclerosis and secondary progressive multiple sclerosis or Crohn's disease. In some embodiments the VLA-blocking agent is CD49d specific, i.e. specific for the integrin α4 chain. Examples of a suitable VLA-4 blocking agent include, but are not limited to, the monoclonal antibodies Natalizumab, HP2/1, HP1/3, HP1/2, including humanized HP1/2, HP1/7, HP2/4, B-5G10, TS2/16, L25, P4C2, AJM300 and the recombinant anti-VLA4 antibodies described in U.S. patents U.S. Pat. No. 6,602,503 and U.S. Pat. No. 7,829,092, a low molecular weight VLA-4 antagonist such as SB-683699, a CS-1 peptidomimetic as disclosed in e.g. U.S. patents U.S. Pat. No. 5,821,231, U.S. Pat. No. 5,869,448, U.S. Pat. No. 5,869,448, U.S. Pat. No. 5,936,065, U.S. Pat. No. 6,265,572, U.S. Pat. No. 6,288,267, U.S. Pat. No. 6,365,619, U.S. Pat. No. 6,423,728, U.S. Pat. No. 6,426,348, U.S. Pat. No. 6,458,844, U.S. Pat. No. 6,479,666, U.S. Pat. No. 6,482,849, U.S. Pat. No. 6,596,752, U.S. Pat. No. 6,667,331, U.S. Pat. No. 6,668,527, U.S. Pat. No. 6,685,617, U.S. Pat. No. 6,903,128 or U.S. Pat. No. 7,015,216, a phenylalanine derivative as disclosed in e.g. U.S. patents U.S. Pat. No. 6,197,794, U.S. Pat. No. 6,229,011, U.S. Pat. No. 6,329,372, U.S. Pat. No. 6,388,084, U.S. Pat. No. 6,348,463, U.S. Pat. No. 6,362,204, U.S. Pat. No. 6,380,387, U.S. Pat. No. 6,445,550, U.S. Pat. No. 6,806,365, U.S. Pat. No. 6,835,738, U.S. Pat. No. 6,855,706, U.S. Pat. No. 6,872,719, U.S. Pat. No. 6,878,718, U.S. Pat. No. 6,911,451, U.S. Pat. No. 6,916,933, U.S. Pat. No. 7,105,520, U.S. Pat. No. 7,153,963, U.S. Pat. No. 7,160,874, U.S. Pat. No. 7,193,108, U.S. Pat. No. 7,250,516 or U.S. Pat. No. 7,291,645, alphafeto protein, a beta-amino acid compound as disclosed in e.g. patent applications US 2004/0229859 or US 2006/0211630, a semi-peptide compound as disclosed in e.g. U.S. Pat. No. 6,376,538, the tripeptide Leu-Asp-Val and a pegylated molecule as disclosed in U.S. patent application US 2007/066533 or U.S. patent U.S. Pat. No. 6,235,711.

In another aspect there is provided a method of treating an autoimmune disease in a subject so as to avoid the additional occurrence of PML. The method includes administering one or more VLA-4 blocking agents to the subject, generally an effective amount of the VLA-4 blocking agent(s), over a period of time, followed by discontinuing the administration for a period of time.

In relation to the method of treatment, the present invention provides the following items:
Item 1. A method of treating a patient comprising administering to the patient VLA-4 blocking agent, detecting the level of T cells expressing L-selectin (CD62L) in a sample from the patient, and continuing or stopping the administration based on the level of expression, wherein the administration is stopped if a decreased level of CD62L expressing T cells, relative to a threshold value, is detected.
Item 2. The method of item 1, wherein the patient suffers from a demyelinating disease or an autoimmune disease.
Item 3. The method of item 2, wherein the VLA-4 blocking agent is an immunoglobulin or a proteinaceous binding molecule with immunoglobulin-like functions.
Item 4. The method of any one of items 1 to 3, further comprising detecting the level of T cells expressing lymphocyte function-associated antigen-1 (LFA-1) in the sample, wherein the administration is stopped if a decreased level of LFA-1 expressing T cells, relative to a threshold value, is detected.
Item 5. The method of any one of items 1 to 4, further comprising further administering to the patient VLA-4 blocking agent the treatment if an increased level of CD62L, relative to the threshold value, is detected after the treatment is stopped.
Item 6. The method according to any one of the preceding items, wherein the method further comprises determining the migration of CD45⁺CD49d⁺ immune cells.
Item 7. The method according to item 6, wherein the immune cells are T cells.
Item 8. The method of any one of items 1 to 7, wherein the sample is one of a blood sample, a blood cell sample, a lymph sample and a sample of cerebrospinal fluid.
Item 9. The method of any one of items 1 to 8, wherein detecting the level of CD62L expressing T cells comprises detecting at least one of:

(i) the number of T cells in the sample from the subject that have CD62L on the cell surface,

(ii) the amount of CD62L present on T cells of the sample from the subject, and

(iii) the amount of nucleic acid formation from the SELL gene encoding CD62Lin T cells of the sample from the subject.
Item 10. The method of any one of items 4 to 9, wherein detecting the level of LFA-1 expressing T cells comprises detecting at least one of:

(i) the number of T cells in the sample from the subject that have LFA-1 on the cell surface,

(ii) the amount of LFA-1 present on T cells of the sample from the subject, and

(iii) the amount of nucleic acid formation from the ITGAL gene encoding CD11A and the ITGB2 gene encoding CD18 in T cells of the sample from the subject.
Item 11. The method of any one of items 1 to 10, wherein the T cells are at least one of CD4⁺ T cells and CD8⁺ T cells.
Item 12. The method of any one of items 1 to 11, comprising repeatedly detecting the level of at least one of CD62L expressing T cells and LFA-1 expressing T cells in a sample from the subject.
Item 13. The method of any one of items 11 to 12, wherein (i) detecting the number of T cells in the sample that have CD62Lon the cell surface and/or (ii) detecting the amount of CD62L present on T cells of the sample comprises contacting T cells in/of the sample with a capture probe, the capture probe being specific for CD62L, and detecting the amount of the capture probe binding to CD62L.
Item 14. The method of any one of items 11 to 16, wherein (i) detecting the number of T cells in the sample that have LFA-1 on the cell surface and/or (ii) detecting the amount of LFA-1 present on T cells of the sample comprises contacting T cells in/of the sample with a capture probe, the capture probe being specific for LFA-1, and detecting the amount of the capture probe binding to LFA-1.

In some embodiments the expression of the one or more biomarkers on T cells, including CD4⁺, CD3⁺ or CD8⁺ T cells are monitored in a sample from the subject. In some embodiments, a respective biomarker is one or more of CD62L and LFA-1. In some embodiments discontinuing the administration of the one or more VLA-4 blocking agents is effected after detection of a decreased or an increased level of the one or more biomarkers on T cells, including CD4⁺, CD3⁺ or CD8⁺ T cells, relative to a threshold value. For example, the level of CD62L expressing T cells in the subject and optionally the level of LFA-1 expressing T cells in the subject may be altered.

Determining the level of CD62L and optionally LFA-1 expressing T cells in any of the above aspects and embodiments may include detecting the number, percentage or level of T cells in the sample from the subject that have CD62L and optionally LFA-1 on the cell surface. Determining the level of CD62L and optionally LFA-1 expressing T cells may also include detecting the amount or level of CD62L and optionally LFA-1 present on T cells of the sample from the subject. Determining the level of CD62L expressing T cells may also include detecting, in T cells of the sample from the subject, the amount or level of nucleic acid formation from the SELL gene encoding CD62L. Determining the level of LFA-1 expressing T cells may also include detecting the amount or level of nucleic acid formation from the ITGAL gene encoding CD11A and/or the ITGB2 gene encoding CD18.

### BRIEF DESCRIPTION OF THE DRAWINGS

The invention will be better understood with reference to the detailed description when considered in conjunction with the non-limiting examples and the accompanying drawings.

FIG. 1A depicts the percentage (%) of CD62L surface expressing CD3⁺ CD4⁺ T cells, as determined by flow cytometric measurements using peripheral blood derived mononuclear cells (PBMC). Cells were isolated from EDTA blood by density gradient centrifugation, frozen, thawed for analysis, and stained with fluorescence labelled immunoglobulins against CD3, CD4 and CD62L. Cells were gated as shown in FIG. 1C. The boxes in FIG. 1A represent 50% of each cohort (25^{th}-75^{th} percentile) while 80% of all individuals reside within the limits of each box and its whiskers (10^{th}-90^{th} percentile). The line within the boxes indicates the mean, the plus (+) represents the median of the respective cohort. Each dot represents an individual patient. The white box represents 21 control subjects without any acute or chronic disorder (healthy controls). The dotted box represents subjects diagnosed for MS, who are in stable condition and did not receive any prior immune-modulating treatment (MS naïve). The light grey box represents patients diagnosed for MS, who received baseline treatments other than Natalizumab as lined-out in FIG. 15. These blood withdrawings took place right before the escalation to Natalizumab therapy (MS baseline). The dark grey box indicates patients diagnosed for MS, who after receiving baseline treatments as lined-out in FIG. 15 received Natalizumab continuously for 18 months or longer (18-66 months of Natalizumab treatment, MS Natalizumab). The six numbered MS (Natalizumab) pre-PML patients all match the criteria of the dark grey cohort, but developed PML later on at different time points throughout Natalizumab long-term therapy as lined out in FIG. 15. The dotted line indicates the threshold for increased PML risk under long-term Natalizumab therapy (mean of the dark grey cohort minus two times its standard deviation).

FIG. 1B depicts the percentage (%) of CD62L surface expressing CD3⁺ CD4⁺ T cells. See the explanation for FIG. 1A for details. The MS (Natalizumab) acute- and post-PML cohorts both match the dark grey cohort but were sampled after PML onset, either while suffering from acute PML (MS (Natalizumab) acute-PML) or after PML subsided (MS (Natalizumab) post-PML, e.g. the beginning of immune reconstitution inflammatory syndrome (IRIS). Two patients with other monoclonal antibody-associated PMLs, one suffering from severe psoriasis treated with Efalizumab and one suffering from B-cell lymphoma treated with Rituximab (other monoclonal antibody-associated acute-PML), and seven HIV/AIDS PML patients (HIV-associated acute-PML) served as additional PML controls. The dashed lines indicate sequential samples, if identical patients were available at different time points during disease development.

FIG. 1C shows illustrative flow cytometry measurements with gating to life lymphocytes, CD3⁺ T cells as well as CD4+ and CD8+ T cells.

FIG. 1D depicts data of flow cytometric measurements of peripheral blood derived mononuclear cells (PBMC). Cells were isolated from EDTA blood (EDTA: 1.2 to 2 mg/ml blood) by density gradient centrifugation and subsequently frozen. For analysis, cells were thawed and stained with fluorescence labelled antibodies against CD3, CD4 and CD62L. 200,000 cells were used per staining. After flow cytometry measurement, cells were first gated to life lymphocytes, then CD3⁺ cells then CD4⁺ cells and finally on CD62L⁺ cells (cf. also FIG. 1C). The graph depicts CD3⁺ CD4⁺ living lymphocytes that are positive for CD62L (1-selectin) expression. The groups are as following: HD=healthy controls without any pathology or treatment; NAT=patients suffering from relapsing/remitting multiple sclerosis long-term treated with Natalizumab (18+ months of treatment); HIV=patients suffering from HIV infection treated with HAART medication; HIV PML=patients suffering from HIV infection treated with HAART medication that developed PML alongside therapy.

FIG. 2 shows percentages of CD14⁺ monocytes, CD4⁺ and CD8⁺ T cells, CD19⁺ B cells, and CD56⁺ NK cells (of PBMC) in peripheral blood of patients receiving long-term Natalizumab therapy (≧18 months). White dots represent healthy donors (n=16-39), black dots represent untreated MS patients (n=12), and grey dots represent Natalizumab patients treated ≧18 months continuously (n=34). Significance of differences between the groups is indicated by asterisks (*p<0.05, **p<0.01, ***p<0.001).

FIG. 3 depicts data analysis of flow cytometric measurements of peripheral blood derived mononuclear cells (PBMC). EDTA blood was obtained from patients and healthy control subjects as indicated above, PBMC were isolated by density gradient isolation and cryo-preserved in 50% RPMI, 40% FCS and 10% DMSO. Samples were subsequently thawed and stained in phosphate buffered saline (200 mM EDTA, 0.5% BSA) for surface markers (CD3, CD4, CD8, and CD62L). 1: Healthy controls, n=73; 2: Untreated RRMS patients, n=12; 3: RRMS patients before Natalizumab therapy, n=30; 4: RRMS patients after long-term Natalizumab therapy, which is defined as a therapy of more than 18 months, n=78; 5: HIV⁻ patients (CDC stadium B1-C2), n=5; 6: HIV⁺ patients (CDC stadium C3), n=9. White circles: RRMS patients under long-term Natalizumab therapy before onset of PML; Black circles: HIV⁺ patients after onset of PML. The percentage of CD62L positive cells of CD3⁺CD4⁺ T cells or CD3⁺CD8⁺ T cells is shown. An isotype control was used to define a threshold between CD62L positive and negative cells.

FIG. 4 shows the immune cell composition in peripheral blood (dots) and CSF (triangles) of patients under Natalizumab therapy (n=18; treatment ≧18 months). Given are percentages of monocytes, CD4⁻ and CD8⁺ T cells and B cells (of total leukocytes).

FIG. 5 shows the in vitro migration of isolated PBMC over primary human microvascular endothelial cells (HBMEC). Given are the absolute values of migrated T cells per μl of sample represented by individual dots of healthy donors (open circles, n=10), untreated MS patients (black, n=16) or Natalizumab patients (grey, n=29). Migration has been assessed after 6 h.

FIG. 6 shows the in vitro migration of isolated PBMC over primary human choroid plexus-derived epithelial cells (HCPEpiC). Given are the absolute values of migrated T cells per μl of sample represented by individual dots of healthy donors (open circles, n=6), untreated MS patients (black, n=6) or Natalizumab patients (grey, n=15). Migration has been assessed after 6 h.

FIG. 7 shows the percentages of LFA-1 expressing T cells before Natalizumab treatment (month 0) and in the time course of therapy (months 1, 3, 6, 12, 15-20, 21-25, 26-30, 31-35, 36-40, 41-45, 46-50, 51-55; n=39 patients). Black symbols indicate the mean calculated from patients at given time points, standard error of the mean are given. The white and grey circles represent two patients who later developed PML.

FIG. 8 shows the percentages of CD62L expressing T cells before Natalizumab treatment (month 0) and in the time course of therapy (months 1, 3, 6, 12, 15-20, 21-25, 26-30, 31-35, 36-40, 41-45, 46-50, 51-55; n=39 patients). Black symbols indicate the mean calculated from patients at given time points, standard error of the mean are given. The white and grey circles represent two patients who later developed PML.

FIG. 9 shows the migration of CD3⁺ T cells (in percent, related to untreated MS patients set to 100%) before Natalizumab treatment (month 0) and in the time course of therapy (months 1, 3, 6, 12, 15-20, 21-25, 26-30, 31-35, 36-40, 41-45, 46-50, 51-55; n=50 patients). Black symbols indicate the mean calculated from patients at given time points, standard error means are given. The white and grey circles represent two patients who later developed PML.

FIG. 10 shows relative quantification of CD11a as compared to hS18 on thawed PBMC from MS patients before (month 0) and in the time course of therapy (months 1, 3, 6, 12, 15-20, 21-25, 26-30, 31-40, 41-50; n=27 patients) as assessed by real-time PCR. Lower delta CT values indicate a higher expression of the target.

FIG. 11 shows relative quantification of Runx-3 as compared to hS 18 on thawed PBMC from MS patients before (month 0) and in the time course of therapy (months 1, 3, 6, 12, 15-20, 21-25, 26-30, 31-40, 41-50; n=28 patients) as assessed by real-time PCR. Lower delta CT values indicate a higher expression of the target.

FIG. 12 shows relative quantification of CD62L as compared to hS18 on thawed PBMC from MS patients before (month 0) and in the time course of therapy (months 1, 3, 6, 12, or more; n=28 patients) as assessed by real-time PCR. Lower delta CT values indicate a higher expression of the target.

FIG. 13 depicts dot plots of samples of the six MS Natalizumab pre-PML patients and one exemplary MS patient before the start of Natalizumab therapy. The numbering of pre-PML patients is in line with the numbering used in FIG. 15. PBMC were first gated on “live lymphocytes”, then CD3⁺ (T cells), then CD4⁺ and finally plotted on CD62L vs. CD45RA to illustrate the loss of CD62L (especially striking on the CD45RA⁺ (naive) CD4⁺ T cells).

FIG. 14 lists the monoclonal antibodies used for flow cytometry.

FIG. 15 lists all patients included in this study. Given are cohort/patient, number of patients, year of birth, sex, first manifestation of MS, EDSS, pre-treatments, JCV antibody seropositivity, cycles of Natalizumab, % CD62L of CD4⁺ T cells (mean, standard deviation, and 10-90 percentile) of the following cohorts: Healthy controls, MS (naïve), MS (baseline treatments), MS (Natalizumab), MS (Natalizumab) pre-PML, MS (Natalizumab) acute-PML, MS (Natalizumab) post-PML, other monoclonal antibody-associated acute-PML and HIV-associated acute-PML, corresponding to the groups in FIG. 1A and FIG. 1B.

### DETAILED DESCRIPTION OF THE INVENTION

It must be noted that, as used herein, the singular forms “a”, “an”, and “the” include plural references unless the context clearly indicates otherwise. Thus, for example, reference to “an antibody” includes one or more of such different antibodies and reference to “the method” includes reference to equivalent steps and methods known to those of ordinary skill in the art that could be modified or substituted for the methods described herein.

The present invention provides methods of determining a prognosis or the risk for PML occurrence. Using a method according to the invention a subject can be identified as being at a higher risk of developing PML when compared to otherwise apparently similar subjects, e.g. subjects of comparable health/disease state or risk factor exposure. In some embodiments a method according to the invention can thus be taken to define a method of assessing the risk level of a subject with regard to PML occurrence. The invention also allows stratifying patients for risk of PML.

The present invention is based on the surprising finding that CD62L levels on T cells can be used as a biomarker for the risk evaluation of occurrence of PML in a subject. In part the present invention is also based on the finding that the binding of VLA-4 influences the expression of the cell surface molecules CD62L and LFA-1 and basic immune cell functions such as migratory capacity. Without being bound by any particular theory, the present inventors have discovered that some cell surface molecules, including CD62L and LFA-1, are differentially expressed on T cells in subjects who/that develop or have developed PML. In addition, the present inventors have found that CD62L is already differently expressed on T cells in subjects who/that are about to develop PML. Use of such molecules as biomarkers, in conjunction with further biomarkers or tests, provides an indication as to which subjects are more likely to suffer from PML. The biomarkers provided in the present invention can assist physicians in determining an appropriate therapeutic regimen.

Accordingly, the biomarkers provided in the present invention can be advantageously used to diagnose the immune competence of a subject, such as a subject who/that is receiving or expected to receive long-term VLA-4 blocking agent treatment, a subject who/that is HIV positive as well as to diagnose the risk of the subject to suffer from PML.

PML is a formerly rare, but severe, subacute, rapidly progressive demyelinating disease of the brain, which was first characterized in 1958. PML has today reached epidemic proportions, mostly due to the fact that HIV/AIDS has resulted in a remarkable increase in the frequency of PML. In some locales, HIV infection has been found to account for more than 90% of the predisposing disorders associated with PML. PML is caused by lytic infection of oligodendrocytes by the John Cunningham virus (JCV), a double-stranded, not enveloped human polyomavirus. JCV infects children, and seropositivity in adults is reported to be between 50% and 60%, with higher prevalence in men than in women (Soelberg Sørensen, P., et al., Multiple Sclerosis Journal (2012) 18, 2, 143-152). The nonenveloped JCV virion is taken up into cells via clathrin dependent receptor-mediated endocytosis. The supposedly transmittable form of JCV has commonly been referred to as the JCV archetype, as it has been assumed that all other genotypes originate from it. These assumptions, are, however, so far not supported by sound evidence, i.e. it is not established whether the transmittable form of JCV is indeed the archetypal form of the virus. It is further not known whether JCV superinfections can occur after initial childhood infection (White, M. K., & Khalili, K., J. Infect. Disease [2011] 203, 5, 578-586). PML is thought to be caused by reactivation of JCV, which can stay latent in a variety of tissues such as the kidneys, the tonsils, B lymphocytes and lymphoid organs as well as the central nervous system. Fragments of JCV DNA have even been found in oligodendrocytes and astrocytes in non-PML brain. The archetypal form of JCV seems to be exclusively found in the kidneys of non-PML individuals. Pathological JCV PML-type variants, which always have, relative to the JCV archetype, an altered regulatory region, form in the host via an unknown mechanism. Compared to the JCV archetype, pathological JCV PML-type variants have also been found to contain in >80% of cases an amino acid substitution in the major capsid protein, VP1, typically in the outer loops. Further, deletions, duplications, and point mutations in the noncoding regulatory region and/or the coding region, have been reported.

JCV causes lytic infection and death of myelin producing oligodendrocytes in the white matter. It also infects astrocytes in a non-productive fashion; an abortive infection can lead to multinucleated giant astrocytes. PML typically results in focal neurologic deficits such as aphasia, hemiparesis and cortical blindness. It is currently diagnosed by analysing cerebrospinal fluid or a brain biopsy specimen for the presence of JCV DNA.

In the context of natalizumab treatment known risk factors for development of PML include the duration of natalizumab exposure, prior immunosuppressive therapy and the presence of anti-JCV antibodies (Soelberg Sørensen et al., 2012, supra). The elevated risks associated with prior use of immunosuppressants, the duration of natalizumab exposure and presence of anti-JCV antibodies appear to be independent of each other. The overall incidence of PML is reported to be about two in 1000 natalizumab-treated patients (ibid.).

One method of the invention is a method of diagnosing or aiding in the diagnosis of the risk of development of a condition associated with JCV in a subject. JCV associated conditions and symptoms of PML generally include defects of motor and/or cognitive performance. Symptoms/conditions that may occur are for instance weakness, hemiparesis, hemiplegia, i.e. partial paralysis, ataxia, altered mental status, visual field disturbances including loss of vision, impaired speech including aphasia, cognitive deterioration, as well as the so called Alien hand syndrome.

A related method of the invention is a method of diagnosing or aiding in the diagnosis of the risk of occurrence of PML in a subject. This method of assessing the risk of occurrence of PML may also be taken as a method of diagnosing the susceptibility of the subject to PML. In this regard the term “susceptibility” as used herein refers to the degree of risk of a subject to an indicated condition, which may be a pathological condition, such as a disease or disorder. The term “susceptibility to PML” refers to the likelihood that PML will occur in a subject. It is understood that a respective diagnosis/assessment involves a valuation which may subsequently turn out to be less than 100% precise for a given individual. Such assessment is in some embodiments to be taken as an indication of the balance of probabilities rather than as a solid predication.

A respective method according to the present invention involves analysis of a sample from the subject in vitro. Typically the sample is, essentially consists of, or includes body fluid from the subject. The term “essentially consists of is understood to allow the presence of additional components in a sample or a composition that do not affect the properties of the sample or a composition. As an illustrative example, a pharmaceutical composition may include excipients if it essentially consists of an active ingredient.

The sample may in some embodiments be one of a whole blood sample, a blood cell sample, a lymph sample and a sample of cerebrospinal fluid. In some embodiments the method may include providing a sample from the subject. The sample may have been taken at any desired point in time before carrying out the method of the invention. Generally time interval between taking the sample and carrying out the method of the invention is selected to allow analysis of viable cells. It is within the skilled artisan's experience to determine a respective time interval during which T cells in a sample can be expected to remain viable. As a general orientation, the inventors have found that in the form of EDTA blood, i.e. after adding a final amount of about 1-2 mg/ml EDTA (typically potassium EDTA), cells remain viable and suitable for carrying out a method according to the invention during a time interval of up to 48 hours during which the sample is kept in fluid form at room temperature, i.e. about 18° C. Cells may for instance be kept at a temperature in the range from about 2° C. to about 37° C., such as from about 4° C. to about 37° C. or below. In some embodiments the sample is kept at about 32° C. or below. In some embodiments the sample is kept at a temperature of about 25° C. or below. As an illustrative example, a whole blood sample may be kept at about 25° C. or below. As a further example a cerebrospinal fluid sample may be kept at about 25° C. or below. As yet a further example a lymph sample may be kept at about 25° C. or below. In some embodiments the sample is kept at a temperature of about 22° C. or below, such as about 18° C. or below. In some embodiments the sample is kept at about 15° C. or below, such as below 10° C. In some embodiments the sample is kept at about 4° C. or at about 8° C. As an illustrative example, a whole blood sample may be kept at about 8° C. or below. As a further example a cerebrospinal fluid sample may be kept at about 8° C. or below.

As used herein, the term “viable” refers to a cell that maintains homeostasis by the use of one or more energy consuming mechanisms. Thus a “viable” cell for example includes a cell in which productive oxidative metabolism occurs to produce the necessary energy; a cell in which only glycolysis is used to produce energy, as well as a cell which maintains cellular integrity, such as the ability to exclude, or actively remove, certain molecules from the interior of the cell, by energy consuming mechanisms. In some embodiments, a viable cell is capable of undergoing mitosis, cell growth, differentiation, and/or proliferation. The expression “viable cell” can be taken to be synonymous with a “living cell”, which includes a cell that is quiescent (and thus not going through the cell cycle), but nonetheless alive because energy production and consumption occurs in such a cell to maintain homeostasis.

In some embodiments the sample has been taken on the same or on the previous day, such as about 48 hours, about 42 hours, about 36 hours, about 30 hours, about 28 hours, about 24 hours, about 18 hours, about 15 hours, about 12 hours, about 10 hours, about 8 hours, about 6 hours or less before the method of the invention is being carried out. In some embodiments the sample has been taken within a period of up to about 48 hours, i.e. 0 to about 48 hours, to about 42 hours, to about 36 hours, to about 30 hours, to about 28 hours, to about 24 hours, to about 18 hours, to about 15 hours or 0 to about 12 hours before the method of the invention is being carried out. The subject, also addressed as a patient or an individual herein, from which/whom the sample has been obtained is an animal, generally a mammal. The sample may be obtained or derived from any animal, including mammalian species such as a rabbit, a mouse, a rat, a Guinea pig, a hamster, a dog, a cat, a pig, a cow, a goat, a sheep, a horse, a monkey, an ape or a human.

In some embodiments the sample from the individual is a frozen sample. Generally the sample is frozen within the above detailed time intervals, e.g. 0 to about 48 or 0 to about 42 hours, and/or at the above exemplified time points, such as about 48 hours, about 36 hours or less, after the sample has been obtained from the individual. A frozen sample may be formed by freezing an obtained sample after adding a cryoprotective agent such as DMSO, glycerol and/or hydroxyethyl starch. In some embodiments, for instance where the sample is a blood cell sample, serum may in addition be added before freezing. As an illustrative example DMSO may be used in a final concentration in the range from about 2% to about 10%, such as about 2%, about 4%, about 5% or about 10% DMSO. Typically the sample is then frozen at a controlled rate to a temperature less than −50° C., whereafter the sample may for instance be stored, including long-term storage, at a temperature below −130° C. such as −160° C., e.g. in liquid nitrogen for extended periods of time.

In some embodiments of a method according to the invention a sample as provided from the individual is depleted of erythrocytes, in some embodiments at least essentially cleared of erythrocytes, if required. Depletion or removal of erythrocytes may for example be required in case the sample is a whole blood sample or a blood cell sample. Lysis of erythrocytes may be carried out osmotically or chemically. Osmotic lysis is suitable in the contest of the present invention since erythrocytes lyse at an osmolarity at which leukocytes remain intact. In the art typically a solution of ammonium chloride is used for osmotic lysis, which may further include potassium bicarbonate and/or EDTA. A commercially available reagent may be used, such as the FCM Lysing solution by Santa Cruz (order no sc-3621), Erythrolyse Red Blood Cell Lysing Buffer by AbD Serotec or RBC Lysis Solution by 5 PRIME. Chemical lysis of erythrocytes may for example be achieved using an organic solvent such as diethylether or chloroform, and/or a surfactant, a copper containing solution or via adding one of certain bacterial or animal toxins. After lysis of erythrocytes the remaining blood cells may be collected, for example by means of centrifugation.

In a method according to the invention, the level of T cells in the sample that have the protein L-selectin and optionally LFA-1 on their surface is detected. T cells are known to the skilled artisan as lymphocytes, i.e. nucleated blood cells that are also called white blood cells. T cells mature in the thymus and can be distinguished from other lymphocytes in that they have the T cell receptor on their cell surface. The main known role of the T cell is recognition of antigens bound to major histocompatibility complex (MHC) molecules. The T cell receptor (TCR) is a heterodimer, which consists of a 34 kD α-chain, linked by a disulphide bond to a 34 kD β-chain in about 95% of T cells. Both chains span the plasma membrane and have accordingly an extracellular portion, each of which includes a variable region, termed Vα and Vβ, respectively. About 5% of T cells have a T cell receptor that consists of a γ- and a δ-chain instead of an α- and a β-chain, which likewise have extracellular variable regions. T cell receptors can, like immunoglobulins, recognize a very large number of different epitopes.

It is to be understood that “level”, “value” and “amount” are used interchangeably in the application to refer to a measurement that is made using any analytical method for detecting the biomarker in a sample and that indicates the presence, absence, absolute amount or concentration, relative amount or concentration, titer, a level, an expression level, a ratio of measured levels, or the like, of, for, or corresponding to the biomarker in the sample. The exact nature of the “value” or “level” depends on the specific design and components of the particular analytical method employed to detect the biomarker.

An “epitope” is antigenic and thus an epitope may also be taken to define an “antigenic structure” or “antigenic determinant”. Thus, a binding domain of an immunoglobulin or of a proteinaceous binding molecule with immunoglobulin-like functions is an “antigen-interaction-site”. The term “antigen-interaction-site” defines, in accordance with the present invention, a motif of a polypeptide, which is able to specifically interact with a specific antigen or a specific group of antigens, e.g. L-selectin and optionally LFA-1 in different species. This binding/interaction is also understood to define a “specific recognition”.

The term “epitope” also refers to a site on an antigen such as CD3, CD4 or CD8, with which an immunoglobulin or a proteinaceous binding molecule with immunoglobulin-like functions forms a complex. In some embodiments, an epitope is a site on a molecule against which an immunoglobulin or a proteinaceous binding molecule with immunoglobulin-like functions will be produced and/or to which an antibody will bind. For example, an epitope can be recognized by an immunoglobulin or a proteinaceous binding molecule with immunoglobulin-like functions. The epitope may be a “linear epitope”, which is an epitope where an amino acid primary sequence contains the epitope recognized. A linear epitope typically includes at least 3, and more usually, at least 5 amino acids in a unique sequence. A linear epitope may for example include about 8 to about 10 amino acids in a unique sequence. The epitope may also be a “conformational epitope”, which in contrast to a linear epitope, is an epitope where the primary sequence of the amino acids that includes the epitope is not the sole defining component of the epitope recognized (e.g., an epitope wherein the primary sequence of amino acids is not necessarily recognized by the antibody defining the epitope). Typically a conformational epitope includes a larger number of amino acids than a linear epitope. With regard to recognition of conformational epitopes, an immunoglobulin or a proteinaceous binding molecule with immunoglobulin-like functions recognizes a 3-dimensional structure of the antigen, such as a peptide or protein or a fragment thereof. As an illustrative example, when a protein molecule folds to form a three dimensional structure, certain amino acids and/or all or portions of the polypeptide backbone forming the conformational epitope become juxtaposed, allowing an antibody to recognize the epitope. Methods of determining conformation of epitopes include, but are not limited to, x-ray crystallography, 2-dimensional nuclear magnetic resonance spectroscopy, site-directed spin labelling and electron paramagnetic resonance spectroscopy.

In some embodiments the presence of the T cell receptor on the surface of a cell may be used to identify the cell as a T cell. As the T cell receptor has variable regions it may, nevertheless, be advantageous to use another cell surface protein to identify a T cell. An example of suitable protein in this regard is a T cell co-receptor. Two illustrative examples of a co-receptor of the T cell receptor are the protein complex CD3 (Cluster of Differentiation 3) and the protein CD247. CD3 has four chains, which are in mammals one D3γ chain, one CD3δ chain, and two CD3ε chains. These chains associate with a molecule known as the T-cell receptor and at least one T-cell surface glycoprotein CD3 zeta chain also known as T-cell receptor T3 zeta chain or CD247 (Cluster of Differentiation 247). CD247 may be present on the cell surface as either a ζ₂ complex or a ζ/η complex. The complex of TCR, CD247 and CD3 can generate an activation signal in T lymphocytes. The TCR, ζ-chain(s), and CD3 molecule together define the TCR complex. In practicing a method according to the invention identifying the presence of CD3 on a particular cell or plurality of cells is often a convenient way of identifying T cells. Therefore the terms “CD3⁺ T cell” and “T cell” are used interchangeable herein to address a T cell and to distinguish a T cell from other cell types.

A further example of a co-receptor of the T cell receptor is the transmembrane protein CD8 (Cluster of Differentiation 8). Most T cells that have CD8 on their surface are cytotoxic T cells. CD8 plays an important role in binding to the class I major histocompatibility complex. Two isoforms of the protein, namely CD8-alpha and -beta, are known. Each such chain contains a domain that resembles an immunoglobulin variable domain. CD8 is a dimer of two of these chains, either a homo- or a heterodimer.

CD4⁺ T cells in addition to CD3 have the CD4 (Cluster of Differentiation 4) protein on their surface, a glycoprotein consisting of four extracellular immunoglobulin domains, termed D₁ to D₄, and a small cytoplasmic region. The CD4 protein is known to be used by HIV-1 to gain entry into T cells of a host. CD4⁺ T cells can be classified into a variety of cell populations with different functions and should thus not be taken to define a unitary set of cells. Typical examples of a CD4⁺ T cell are a T helper cell, a regulatory T cell and a memory T cell.

In some embodiments a method according to the invention includes identifying CD3⁺ T cells in the sample, for example by employing an immunoglobulin, an immunoglobulin fragment or a proteinaceous binding molecule with antibody-like functions as further explained below. By “fragment” in reference to a polypeptide such as an immunoglobulin or a proteinaceous binding molecule is meant any amino acid sequence present in a corresponding polypeptide, as long as it is shorter than the full length sequence and as long as it is capable of performing the function of interest of the protein—in the case of an immunoglobulin specifically binding to the desired target, e.g. antigen (CD62L and optionally LFA-1, for example). The term “immunoglobulin fragment” refers to a portion of an immunoglobulin, often the hypervariable region and portions of the surrounding heavy and light chains that displays specific binding affinity for a particular molecule. A hypervariable region is a portion of an immunoglobulin that physically binds to the polypeptide target.

In some of these embodiments identifying CD3⁺ T cells in the sample serves in distinguishing CD3⁺ T cells from other cells such as CD3⁻ cells or non-T cells. In some embodiments CD4⁺ T cells are identified in the sample. Identifying CD4⁺ T cells typically serves in distinguishing CD4⁺ T cells from other cells such as CD4⁻ T cells or non-T cells. In some embodiments CD8⁺ T cells are identified in the sample. Identifying CD8⁺ T cells typically serves in distinguishing CD8⁺ T cells from other cells such as CD8⁻ T cells or non-T cells. It is understood that CD4⁺ T cells and CD8⁺ T cells are typically also CD3⁺ T cells so that a CD3⁺ T cell identified may also for instance be a CD4⁺ T cell rather than be distinguished from a CD4⁺ T cell. Accordingly, in some embodiments in a first step a T cell may be identified as a CD3⁺ T cell. In a second step it may be determined whether the CD3⁺ T cell is a CD4⁻ T cell. It may also be determined whether the CD3⁺ T cell is a CD8⁺ T cell. In some embodiments T cells are identified by the presence of CD3. Of the thus identified T cells CD4⁺ T cells are distinguished from CD8⁺ T cells.

In some embodiments a method according to the invention includes enriching and/or isolating CD3⁺ T cells from the sample. In some embodiments a method according to the invention includes enriching and/or isolating CD4⁺ T cells and/or CD8⁺ T cells from the sample. In some embodiments T cells are enriched, including sorted, based on the presence of CD3 on the cell surface. Of the thus enriched T cells, those T cells that have CD4 on their surface, i.e. CD4⁺ T cells, may be further enriched. In some embodiments, of the T cells that have been enriched based on the presence of CD3, those T cells that have CD8 on their surface, i.e. CD8⁺ T cells, may be further enriched. As an illustrative example, the sample may be from an individual undergoing treatment with a VLA-4 blocking agent. CD3⁺ T cells may be enriched in a first step, of which CD4⁺ T cells may be enriched in a second step, thereby obtaining enriched CD3⁺ CD4⁺ T cells. The CD3⁺ CD4⁺ T cells of the individual undergoing treatment with a VLA-4 blocking agent may then be used in a method according to the invention. Furthermore, CD8⁺ T cells may be enriched in a second step, thereby obtaining enriched CD3⁺CD8⁻ T cells from the individual undergoing treatment with a VLA-4 blocking agent. CD8 positive T cells or CD4 positive T cells of the individual may for instance be used to analyse the expression of CD62L thereon.

In some embodiments enriching and/or isolating CD3⁺ T cells, CD4⁺ T cells and/or CD8⁺ T cells from the sample includes cell sorting and/or selection, for instance via negative magnetic immunoadherence or flow cytometry. In some embodiments enriching and/or isolating such cells consist of cell sorting or selection. Such a technique may be based on contacting the cells with a plurality of antibodies directed to cell surface markers present on the cells negatively selected. As an illustrative example, to enrich for CD4⁺ cells by negative selection, a plurality of antibodies may include antibodies directed to CD14, CD20, CD11b, CD16, HLA-DR, and CD8, while to enrich for CD8⁺ cells by negative selection, a plurality of antibodies may include antibodies directed to CD14, CD20, CD11b, CD16, and HLA-DR.

In some embodiments it may be desired to enrich for or positively select for T cells that express CD3⁺. In some embodiments undesired cells are depleted by contacting them with particles/beads on which antibodies are immobilized that bind to proteins found on undesired cells, but not on desired cells. In some embodiments desired cells are collected from the sample by contacting them with beads on which antibodies are immobilized that bind to proteins found on the desired cells, but not on undesired cells.

For isolation of a desired population of cells by positive or negative selection, the amount and concentration of cells and particle/bead surface can be varied. In certain embodiments it may be desired to reduce the volume in which beads and cells are contacted, for instance to ensure maximum contact of cells and beads. For example, in one embodiment, a concentration of about 2 billion cells/ml is used. In one embodiment, a concentration of about 1 billion cells/ml is used. In a further embodiment, a concentration of more than about 100 million cells/ml is used. In some embodiments a concentration of cells of about 10 million cells/ml or more is used. In some embodiments cells are at a concentration of about 15, including about 20, about 25 or about 30 million cells/ml. In some embodiments a concentration of cells of about 35, about 40, about 45, about 50 million cells/ml or more is used. In some embodiments a concentration of cells of about 75 million cells/ml is used. In some embodiments cells are at a concentration of about 80 million cells/ml. In some embodiments cells are at a concentration of about 85 million cells/ml. The concentration of cells may for example be about 90, including about 95, about 100, or about 125 million cells/ml or more. In some embodiments a concentration of cells of about 150 million cells/ml or more is used. The use of high cell concentrations may in some embodiments result in increased cell yield, cell activation, and cell expansion. In some embodiments the use of high cell concentrations may allow more efficient capture of cells that may express e.g. CD62L in low number.

The word “about” as used herein refers to a value being within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. The term “about” is also used to indicate that the amount or value in question may be the value designated or some other value that is approximately the same. The phrase is intended to convey that similar values promote equivalent results or effects according to the invention. In this context “about” may refer to a range above and/or below of up to 10%. The word “about” refers in some embodiments to a range above and below a certain value that is up to 5%, such as up to up to 2%, up to 1%, or up to 0.5% above or below that value. In one embodiment “about” refers to a range up to 0.1% above and below a given value.

Those skilled in the art will recognize or be able to ascertain, using no more than routine experimentation, many equivalents to the specific embodiments of the invention described herein. Such equivalents are intended to be encompassed by the present invention. Throughout this specification and the claims which follow, unless the context requires otherwise, the word “comprise”, and variations such as “comprises” and “comprising”, will be understood to imply the inclusion of a stated integer or step or group of integers or steps, but not the exclusion of any other integer or step or group of integer or step. Several documents are cited throughout the text of this specification. Each of the documents cited herein (including all patents, patent applications, scientific publications, manufacturer's specifications, instructions, etc.) is incorporated by reference in their entirety. Nothing herein is to be construed as an admission that the invention is not entitled to antedate such disclosure by virtue of prior invention.

Progressive multifocal encephalopathy is a fatal demyelinating disease of the central nervous system (CNS), caused by the lytic infection of oligodendrocytes by a human polyomavirus, JC virus (JCV). PML is rare disease which mostly develops in patients with underlying immunosuppressive conditions, including Hodgkin's lymphoma, lymphoproliferative diseases and in those undergoing antineoplastic therapy and AIDS. As discussed earlier, PML is also associated with demyelinating disease or autoimmune disease patients including multiple sclerosis and Crohn's disease who are treated with antibody-based regimens (natalizumab, efalizumab and rituximab). This indicates the existence of a strong link between the underlying immunosuppressive conditions and development of PML. MRIs of the brain can detect active disease and, in some cases, in the preclinical state. However, given the rapid progression of PML, scans would have to be taken frequently to provide hope of earlier detection. Since PML is a potentially fatal disease with no specific therapy available, there is a pressing need for a method screening for patients at increased risk for PML.

To address this need, the present invention provides a method of assessing an individual's immune competence and susceptibility to PML. This is also described herein as assessing the “risk of occurrence” of PML for an individual. As used herein, the term “susceptibility” or “risk of occurrence” refers to the degree of risk of a subject to a given disease or pathological condition. The term susceptibility or risk of occurrence to PML refers to the likelihood of a subject to develop or suffer from PML, particularly if the patient continues with the treatment. As will be understood by those skilled in the art, such determination is usually not intended to be correct for 100% of the subjects to be analyzed.

According to the present invention, the term “patient” or “subject” refers to animals, preferably mammals, and more preferably, humans. For the purpose of the present application, a “patient undergoing VLA4 blocking agent treatment” is defined as a patient who is expected to receive, receiving or having received VLA-4 blocking agent. VLA-4 (Very Late Antigen-4, also termed Integrin α4β1) is expressed on the surface of all leukocytes except neutrophils. It recognizes the vascular cell adhesion molecule-1(VCAM-1), an inducible cell surface molecule which mediates the adhesion of lymphocytes, monocytes, eosinophils, and basophils to vascular endothelium. VCAM-1 binds to the VLA-4 when the leukocytes are activated by chemotactic agents or other stimuli, and mediates leukocyte extravasion to sites of tissue inflammation. Structurally, VLA-4 is a heterodimer composed of CD49d (α4) and CD29 (β1) (Hemler et al, “Structure of the Integrin VLA-4 and Its Cell-Cell and Cell Matrix Adhesion Functions,” 1990, Immunological Reviews, 114:45-65.

A VLA-4 blocking agent is defined as a molecule which binds to VLA-4 antigen on the surface of a leukocyte with sufficient specificity to inhibit the VLA-4NCAM-1 interaction. In a preferred embodiment, the blocking agent binds to VLA-4 integrin with a Kd of less than 10⁻⁶ M. VLA-4 blocking agents may be a VLA-4 binding antibody (a full length VLA-4 binding antibody) or VLA-4 binding antibody fragment, such as a Fab fragment, a F(ab′)₂ fragment, a bivalent fragment including two Fab fragments linked by a disulfide bridge at the hinge region, a Fd fragment consisting of the VH and CHI domains, a Fv fragment consisting of the VL and VH domains of a single arm of an antibody, a dAb fragment which consists of a VH domain), or an isolated complementarity determining region (CDR) that retains functionality.

VLA-4 blocking agents inhibit the migration of leukocytes from the blood to the central nervous system by disrupting adhesion between the T-cell and endothelial cells. This is believed to result in the reduction of proinflammatory cytokines, and thus the reduction of the occurrence of pathologic inflammatory disease within the CNS.

VLA-4 blocking agents include, besides Natalizumab (Biogen U.S. Pat. No. 5,840,299), MAbs HP2/1, HP1/3 (Elices et al, 1990, “VCAM-1 on Activated Endothelium Interacts with the Leukocyte Integrin VLA-4 at a Site Distinct from the VLA-4/Fibronectin Binding Site”, Cell 60:577-584), HP1/2 (Sanchez-Madrid et al, 1986, “VLA-3: A novel polypeptide association within the VLA molecular complex: cell distribution and biochemical characterization,” Eur. J. Immunol, 16:1343-1349), humanized HP1/2 (U.S. Pat. No. 6,602,503), HP1/7, HP2/4, B-5G10, TS2/16 (Pulido et al, 1991, “Functional Evidence for Three Distinct and Independently Inhibitable Adhesion Activities Mediated by the Human Integrin VLA-4,” J Biol. Chem. 266(16):10241-5), mAB L25 (Becton Dickinson GmBH, Germany), P4C2 (Abcam, Cambridge, UK), and AJM300 (Ajinomoto, Japan), and recombinant anti-VLA4 antibodies as described in U.S. Pat. No. 6,602,503 and U.S. Pat. No. 7,829,092. one embodiment, the VLA-blocking agent is CD49d(α4) specific.

Furthermore, the VLA-4 blocking agents may also be VLA-4 antagonists that are not monoclonal antibodies, such as SB-683699 (GlaxoSmithKline, Middlesex, UK),a small molecule dual a4 antagonist and small molecules CS-1 peptidomimetics (U.S. Pat. Nos. 5,821,231, 5,869,448, 5,869,448; 5,936,065; 6,265,572; 6,288,267; 6,365,619; 6,423,728; 6,426,348; 6,458,844; 6,479,666; 6,482,849; 6,596,752; 6,667,331; 6,668,527; 6,685,617; 6,903,128; and 7,015,216), phenylalanine derivatives (U.S. Pat. Nos. 6,197,794; 6,229,011; 6,329,372; 6,388,084; 6,348,463; 6,362,204; 6,380,387; 6,445,550; 6,806,365; 6,835,738; 6,855,706; 6,872,719; 6,878,718; 6,911,451; 6,916,933; 7,105,520; 7,153,963; 7,160,874; 7,193,108; 7,250,516; and 7,291,645) alphafeto protein (U.S. Pat. Pub. No. 2010/0150915), Beta-amino acid compounds (U.S. Pat. Pub. Nos. 2004/0229859 and 2006/0211630), semi-peptide compounds (U.S. Pat. No. 6,376,538), Leu-Asp-Val tripeptide (U.S. Pat. No. 6,552,216), pegylated molecules as described in (U.S. Pat. Pub. No. 2007/066533, U.S. Pat. No. 6,235,711).

In some embodiments, the patient is diagnosed with multiple sclerosis, including relapsing-remitting multiple sclerosis (RRMS) and secondary progressive multiple sclerosis (SPMS), as well as Crohn's disease and other autoimmune diseases. The term “treatment” as used herein, means to reduce, stabilize, or inhibit progression of a symptom which are associated with the presence and/or progression of a disease or pathological condition

Isolation of a desired population of cells may in some embodiments include general cell enrichment techniques such as centrifugation, filtration or cell chromatography. Generally, isolating or enriching a desired population of cells may be carried out according to any desired technique known in the art. In some embodiments isolation of a desired population of cells may include the use of a commercially available cell isolation kit. T cells may for instance be obtained from peripheral blood, from blood, cerebrospinal fluid, or enriched fractions thereof. T cells may for instance be obtained from peripheral blood mononuclear cells (PBMC) such as human PBMCs. In some embodiments PBMC may for instance be enriched using a standard technique based on cell density and/or cell size. As an illustrative example, PBMC may be enriched or isolated via density gradient centrifugation, for example using sucrose, dextran, Ficoll® or Percoll®. T cells may then be enriched or purified from the obtained PBMCs, for example using a commercially available T cell isolation kit such as the Dynabeads® Untouched™ Human CD4 T Cells kit available from Invitrogen or the StemSep® Human CD4⁺ T Cell Enrichment Kit from STEMCELL Technologies Inc.

The term “purified” is understood to be a relative indication in comparison to the original environment of the cell, thereby representing an indication that the cell is relatively purer than in the natural environment. It therefore includes, but does not only, refer to an absolute value in the sense of absolute purity from other cells (such as a homogeneous cell population). Compared to the natural level, the level after purifying the cell will generally be at least 2-5 fold greater (e.g., in terms of cells/ml). Purification of at least one order of magnitude, such as about two or three orders, including for example about four or five orders of magnitude is expressly contemplated. It may be desired to obtain the cell at least essentially free of contamination, in particular free of other cells, at a functionally significant level, for example about 90%, about 95%, or 99% pure. With regard to a nucleic acid, peptide or a protein, the above applies mutatis mutandis. In this case purifying the nucleic acid, peptide or protein will for instance generally be at least 2-5 fold greater (e.g., in terms of mg/ml).

The term “isolated” indicates that the cell or cells or the peptide(s) or nucleic acid molecule(s) has/have been removed from its/their normal physiological environment, e.g. a natural source, or that a peptide or nucleic acid is synthesized. Use of the term “isolated” indicates that a naturally occurring sequence has been removed from its normal cellular (i.e., chromosomal) environment. Thus, the sequence may be in a cell-free solution or placed in a different cellular environment. Thus, a cell or cells may be included in a different medium such as an aqueous solution than provided originally, or placed in a different physiological environment. By “isolated” in reference to a polypeptide or nucleic acid molecule is meant a polymer of amino acids (2 or more amino acids) or nucleotides coupled to each other, including a polypeptide or nucleic acid molecule that is isolated from a natural source or that is synthesized. The term “isolated” does not imply that the sequence is the only amino acid chain or nucleotide chain present, but that it is essentially free, e.g. about 90-95% pure or more, of e.g. non-amino acid material and/or non-nucleic acid material, respectively, naturally associated with it.

By the use of the term “enriched” in reference to a polypeptide, a nucleic acid or a cell is meant that the specific amino acid/nucleotide sequence or cell, including cell population, constitutes a significantly higher fraction (2-5 fold) of the total amino acid sequences or nucleic acid sequence present in the sample of interest than in the natural source from which the sample was obtained. The polypeptide, a nucleic acid or a cell may also constitute a significantly higher fraction than in a normal or diseased organism or than in normal or diseased cells or in the cells from which the sequence was taken. This could be caused by preferential reduction in the amount of other amino acid/nucleotide sequences or cells present, or by a preferential increase in the amount of the specific amino acid/nucleotide sequence or cell of interest, or by a combination of the two. However, it should be noted that enriched does not imply that there are no other amino acid sequences, nucleotide sequences or cells present. The term merely defines that the relative amount of the sequence of interest has been significantly increased. The term significant here is used to indicate that the level of increase is useful to the person achieving such an increase, and generally means an increase relative to other amino acid sequences of about at least 2-fold, for example at least about 5- to 10-fold or even more. The term is meant to cover only those situations in which man has intervened to increase the proportion of the desired amino acid sequence, nucleotide sequence or cell.

Where desired, further matter may be added to the sample for analysis, for example dissolved or suspended in the sample. It is understood that any dilution due to such addition of matter has to be accounted for and may need to be considered when calculating the level of L-selectin (CD62L) expressing T cells, including CD4⁺ or CD8⁺ T cells. As an illustrative example one or more buffer compounds may be added to the sample. Numerous buffer compounds are used in the art and may be used to carry out the various methods described herein. Examples of buffers include, but are not limited to, solutions of salts of phosphate, carbonate, succinate, carbonate, citrate, acetate, formate, barbiturate, oxalate, lactate, phthalate, maleate, cacodylate, borate, N-(2-acetamido)-2-amino-ethanesulfonate (also called (ACES), N-(2-hydroxyethyl)-piperazine-N′-2-ethanesulfonic acid (also called HEPES), 4-(2-hydroxyethyl)-1-piperazine-propanesulfonic acid (also called HEPPS), piperazine-1,4-bis(2-ethanesulfonic acid) (also called PIPES), (24[tris(hydroxymethyl)-methylamino]-1-ethansulfonic acid (also called TES), 2-cyclohexyl-amino-ethansulfonic acid (also called CHES) and N-(2-acetamido)-iminodiacetate (also called ADA). Any counter ion may be used in these salts; ammonium, sodium, and potassium may serve as illustrative examples. Further examples of buffers include, but are not limited to, triethanolamine, diethanolamine, ethylamine, triethylamine, glycine, glycylglycine, histidine, tris(hydroxymethyl)aminomethane (also called TRIS), bis-(2-hydroxyethyl)-imino-tris(hy-droxymethyl)methane (also called BIS-TRIS), and N-[Tris(hydroxymethyl)-methyl]-glycine (also called TRICINE), to name a few. A respective buffer may be an aqueous solution of such buffer compound or a solution in a suitable polar organic solvent. Further examples of matter that may be added to the sample include salts, detergents or chelating compounds. As yet a further illustrative example, nuclease inhibitors may need to be added in order to maintain a nucleic acid molecule in an intact state.

In an embodiment of a method according to the invention the level of L-selectin (CD62L) expressing CD3⁺ T cells in the sample is detected. The terms “expression”, “gene expression” or “expressing” as used herein relate to the entirety of regulatory pathways converting the information encoded in the nucleic acid sequence of a gene first into messenger RNA (mRNA) and then to a protein. Accordingly, the expression of a gene includes its transcription into a primary hnRNA, the processing of this hnRNA into a mature RNA and the translation of the mRNA sequence into the corresponding amino acid sequence of the protein. In this context, it is also noted that the term “gene product” refers not only to a protein, including e.g. a final protein (including a splice variant thereof) encoded by that gene and a respective precursor protein where applicable, but also to the respective mRNA, which may be regarded as the “first gene product” during the course of gene expression.

The protein L-selectin may be any respective variant or isoform of the respective species, e.g. human. The protein may for example be the human protein of the Swissprot/Uniprot accession number P14151 (version 145 as of 22 Feb. 2012) or the human protein of the Swissprot/Uniprot accession number Q9UJ43 (version 97 as of 22 February 2012). This protein may for instance be encoded by the SELL gene of GenBank accession number NG_{—}016132 (version NG_{—}016132.1 as of 1 Feb. 2012; GI:270047500). The protein may for example be encoded by the mRNA of GenBank accession number BC020758 (version BCO20758.1 as of 4 Aug. 2008; GI: 18088807). The protein may in some embodiments be the mouse protein of the Swissprot/Uniprot accession number P18337 (version 116 as of 22 Feb. 2012). In some embodiments the protein may be the rat protein of the Swissprot/Uniprot accession number P30836 (version 94 as of 22 Feb. 2012) or the rat protein of the Swissprot/Uniprot accession number Q63762 (version 89 as of 22 Feb. 2012). The protein may also be the bovine protein of the Swissprot/Uniprot accession number P98131 (version 82 as of 22 Feb. 2012) or the bovine protein of the Swissprot/Uniprot accession number F1N4U9 (version 8 as of 22 Feb. 2012).

As discussed earlier, PML is observed in patients administered with the VLA-4 blocking agent Natalizumab. It is still unclear how such treatment interferes with JC virus control. Due to the few biomarkers available to identify patients at risk for developing PML, and that the benefit of such treatment outweighs the risk in most cases, there is an urgent need for a test which preemptively stratifies patients who are more prone to PML. As used herein, a “biomarker” is a substance or a condition whose detection indicates a particular biological state, such as a weakened immune competence.

From another perspective, the present invention provides a method of screening patients who are more susceptible to PML. The term “screen” or “screening” as used herein refers to the process of determining the susceptibility, propensity, risk, or risk assessment of a subject for having or developing a disorder. The method of the present invention comprises monitoring the biomarker expressed in a sample from a patient. The term “monitor,” as used herein, generally refers to the overseeing, supervision, regulation, watching, tracking or surveillance of an activity. For example, the term “monitoring the expression of CD62L” refers to measuring and following the expression of mRNA or protein of CD62L over time at predetermined time intervals.

The present invention provides a novel biomarker for the risk evaluation for patients undergoing VLA-4 blocking agent. The inventors have surprisingly discovered that the change in the level of mRNA or protein expression of the biomarkers presently provided can be associated with a weakened immune state and a susceptibility to PML. One of the novel biomarkers is CD62L protein, also known as L-selectin and SELL. It is a cell adhesion molecule found on leukocytes which has been described to be one of the key molecules involved in the homing of T cells to the secondary lymphoid organs. CD62L mediates lymphocyte homing to high endothelial venules of peripheral lymphoid tissue and leukocyte rolling on activated endothelium at inflammatory sites. Most peripheral blood B cells, T cells, monocytes and granulocytes express CD62L/L-selectin. However, some natural killer cells, spleen lymphocytes, bone marrow lymphocytes, bone marrow myeloid cells, thymocytes, and certain hematopoietic malignant cells also express CD62L. Its expression is commonly used to differentiate between central- and effector-memory T cells.

The expression of the biomarkers disclosed herein can be measured prior to, at the same time, or at an early stage of the treatment of VLA-4 blocking agents. To monitor the expression of the biomarker, a sample is first isolated from the patient. The sample may be any bodily fluid. Preferably, peripheral blood is used so mononuclear blood cells can be obtained. Peripheral mononuclear blood cells can be isolated using any techniques known to a skilled person in the art, such as density gradient centrifugation using lymphocyte separation medium (PAA Laboratories, Pasching, Austria) or Ficoll-Paque (Amerisham biosciences, Uppsala, Sweden).

It is to be understood that the term “expression” as used herein refers to the transcription from a gene to give an RNA, as well as the translation from the RNA molecule to give a protein.

The processes of isolating T cells are not limited to any particular technique. Preferably, T cells can be isolated depending upon cell density, affinity of antibody against cell surface epitope, cell size, and/or degree of fluorescent emission. For example, T cells may be isolated by conducting a density gradient centrifugation using albumin, dextran, Ficoll, metrizamid, Percoll, MACS® (Miltenyi Biotec, Bergisch Gladbach, Germany) and further identified using appropriate antibodies, such as anti-CD4 T cell antibody. Centrifugal elutriation, cell size, FACS using fluorescence, magnetic bead isolation or other methods known in the art may also be used.

Expression may be identified by any method known in the art. Needless to say, the expression monitored can be, for example, mRNA expression or protein expression of the biomarkers. Any method for determining biomarker expression may be used to compare the expression level. Suitable methods include immunoassays, such as competitive and non-competitive assay systems, using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, and protein A immunoassays. The assays will develop a signal which is indicative for the presence or absence of the biomarkers. The signal strength can be correlated directly or indirectly (for example, reverse-proportional) to the amount of polypeptide present in a sample. Other suitable method measuring a physical or chemical property specific for the protein is precise molecular mass or NMR spectrum. The methods may include, biosensors, optical devices coupled to immunoassays, biochips, analytical devices such as mass-spectrometers, NMR-analyzers, or chromatography devices. Further methods include microplate ELISA-based methods, fully automated or robotic immunoassays (such as ELECSYS analyzers from Roche Diagnostics), CBA (an enzymatic cobalt binding assay) and latex agglutination assays.

Provided herein is also the use of mRNAs as biomarkers to monitor the patient's immune competence during the course of treatment with VLA-4 blocking agents. For example, the mRNA level of CD62L and optionally LFA-1 can be used to determine whether the treatment might lead to the development of PML. The method provided herein is useful for the initial evaluation of whether to such treatment should be pursued. The method is also useful for following the progress of the treatment and enabling a timely withdrawl, should any loss or deterioration of immune incometence is detected. The biomarkers of the present invention may also be used to track the effectiveness of the treatment and to gather information needed to make adjustments in the patient's treament by, for example, decreasing or even increasing the dose as needed.

The determination of expression may be based on the normalized expression level of the biomarkers. Expression levels are normalized by correcting the absolute expression level of a biomarker by comparing its expression to the expression of a gene that is not a biomarker. Alternatively, the expression level can be provided as a relative expression level.

In an embodiment of a method according to the invention, the level of LFA-1 expressing T cells in the sample is detected. LFA-1 is an integrin-type cell adhesion molecule that is predominantly involved in leukocyte trafficking and extravasation. LFA-1 binds to CD54, the Intercellular Adhesion Molecule 1, on antigen-presenting cells. LFA-1 is a heterodimer having a β-chain, termed CD18, and an α-chain, termed CD11a. Both the α-chain and the β-chain contain a von Willebrand factor type A domain (VWFA domain) in their N-terminal portion, also called inserted domain (I-domain). that plays a central role in regulating ligand binding. Also known as CD11a/CD18, LFA-1 plays a crucial role in many cellular and immunological processes (migration, antigen presentation, cytotoxicity, cell proliferation and haematopoiesis) by displaying both signaling and adhesive properties.

The protein CD18 may be any respective variant or isoform of the respective species, e.g. human. In some embodiments CD18 is the human protein of the Swissprot/Uniprot accession number Swissprot/Uniprot accession number P05107 (version 164 as of 22 Feb. 2012) or the human protein of the Swissprot/Uniprot accession number B4E021 (version 24 as of 22 Feb. 2012). The protein may also be the goat protein of the Swissprot/Uniprot accession number Q5VI41 (version 40 as of 25 Jan. 2012), the porcine protein of the Swissprot/Uniprot accession number P53714 (version 85 as of 22 Feb. 2012) or the bovine protein of the Swissprot/Uniprot accession number P32592 (version 101 as of 22 Feb. 2012). CD18 may be the protein encoded by the ITGB2 gene, for example the mouse gene of GenBank Gene ID No 12575 as of 19 Feb. 2012 or the human gene of GenBank Gene ID No 3689 as of 19 Feb. 2012.

The protein CD45 may be any respective variant or isoform of the respective species, e.g. human. The protein may for example be the human protein of the Swissprot/Uniprot accession number P20701 (version 137 as of 22 Feb. 2012) or the human protein of the Swissprot/Uniprot accession number Q96HB 1 (version 76 as of 22 Feb. 2012). The protein may also be the mouse protein of the Swissprot/Uniprot accession number P24063 (version 108 as of 22 Feb. 2012) or the bovine protein of the Swissprot/Uniprot accession number P61625 (version 56 as of 22 Feb. 2012). CD45 may be the protein encoded by the ITGAL gene, such as the human gene of GenBank Gene ID No 3683 as of 5 Feb. 2012, the bovine gene of GenBank Gene ID No 281874 as of 4 Feb. 2012 or the mouse gene of GenBank Gene ID No 16408 as of 14 Feb. 2012.

The word “detect” or “detecting” refers to any method that can be used to detect the presence of a nucleic acid (DNA and RNA) or a protein/polypeptide. When used herein in combination with the words “level”, “amount” or “value” the word “detect” or “detecting” is understood to generally refer to a quantitative rather than a qualitative level. Accordingly, the method includes a quantification of CD62L and optionally LFA-1—i.e. the amount or number of CD62L expressing and optionally LFA-1 expressing T cells, e.g. CD3 positive T cells, is determined. In this regard the words “value,” “amount” and “level” are used interchangeably herein. The terms “value,” “amount” and “level” also refer to the rate of synthesis of CD62L and optionally LFA-1 in CD3⁺ T cells, as explained further below. The rate of synthesis of CD62L may for example be assessed by determining the synthesis rate of messenger RNA (mRNA) encoded by the selectin L (SELL) gene. Synthesis of CD62L mRNA refers to any mRNA transcribed from a SELL gene (e.g. GenBank accession No. NG_{—}016132, version NG_{—}016132.1, GI: 70047500). Currently two transcript variants of human SELL are known, termed variant 1 (GenBank accession No. NM_{—}000655, version NM_{—}000655.4, GI:262206314) and variant 2 (GenBank accession No. NR_{—}029467, version NR_{—}029467.1; GI:262205323). Synthesis of CD18 mRNA refers to any mRNA transcribed from a ITGB2 gene. Synthesis of CD45 mRNA refers to any mRNA transcribed from a ITGAL gene.

The rate of synthesis of LFA-1 may in some embodiments be detected by determining the synthesis rate of mRNA encoded by the ITGAL gene and the ITGB2 gene. Synthesis of ITGAL mRNA refers to any mRNA transcribed from an ITGAL gene. Currently two transcript variants of the human integrin alpha L gene are known, termed variant 1 (GenBank accession No. NM_{—}002209, version NM_{—}002209.2, GI:167466214) and variant 2 (GenBank accession No. NM_{—}001114380, version NM_{—}001114380.1; GI:167466216). Human mRNA of the human ITGAL gene may also have or include the sequence of GenBank accession No. BC008777 (version BC008777.2, GI:33870544). Four transcript variants of the mouse ITGAL gene are known, termed variant 1 (GenBank accession No. NM_{—}001253872, version NM_{—}001253872.1, GI:359751454), variant 2 (GenBank accession No. NM_{—}008400, version NM_{—}008400.3; GI:359751456), variant 3 (GenBank accession No. NM_{—}001253873, version NM_{—}001253873.1; GI:359751457) and variant 4 (GenBank accession No. NM_{—}001253874, version NM_{—}001253874.1; GI:359751459). Further illustrative examples of ITGAL mRNA the synthesis rate of which may be analyzed, are dog mRNA with the sequence of GenBank accession No. XM_{—}547024 (version XM_{—}547024.2, GI:73958404), wild boar mRNA with the sequence of GenBank accession No. EF585976 (version EF585976.1, GI:156601155) and rat mRNA with the sequence of GenBank accession No. BC101849 (version BC101849.1, GI:74353690).

Synthesis of ITGB2 mRNA refers to any mRNA transcribed from an ITGB2 gene. Currently two transcript variants of the human integrin beta 2 gene are known, termed variant 1 (GenBank accession No. NM_{—}000211, version NM_{—}000211.3, GI:188595673) and variant 2 (GenBank accession No. NM_{—}001127491, version NM_{—}001127491.1; GI:188595676). Human mRNA of the human ITGAL gene may also have or include the sequence of GenBank accession No. S75297 (version S75297.1; GI:242219). Further examples of ITGB2 mRNA, the synthesis of which may be determined, include, but are not limited to, mouse mRNA with the sequence of GenBank accession No. NM_{—}008404 (version NM_{—}008404.4, GI:145966904), rat mRNA with the sequence of GenBank accession No. NM_{—}001037780 (version NM_{—}001037780.2, GI:163937848), dog mRNA with the sequence of GenBank accession No. XM_{—}849290 (version XM_{—}849290.3, GI:359323519) and chicken mRNA with the sequence of GenBank accession No. NM_{—}205251 (version NM_{—}205251.1, GI:46048727).

In the context of the present invention the terms “detect” or “detecting” typically refer to a method that can be used to determine the amount of a nucleic acid or a protein or from which such an amount can be inferred. Examples of such methods include, but are not limited to, RT-PCR, RNAse protection assay, Northern analysis, Western analysis, ELISA, radioimmunoassay or fluorescence titration assay. The detection method may include an amplification of the signal caused by the nucleic acid or protein, such as a polymerase chain reaction (PCR) or the use of the biotin-streptavidin system, for example in form of a conjugation to an immunoglobulin, as also explained in more detail below. The detection method may for example include the use of an immunoglobulin, which may be linked to an attached label, such as for instance in Western analysis or ELISA. Where desired, an intracellular immunoglobulin may be used for detection. Some or all of the steps of detection may be part of an automated detection system. Illustrative examples of such systems are automated real-time PCR platforms, automated nucleic acid isolation platforms, PCR product analysers and real-time detection systems.

The rate of synthesis of CD62L, CD18 and/or CD45 may also be assessed by determining the synthesis rate of the respective protein/polypeptide, including the post-translational modifications of the initial translation product. CD62L is for example synthesized in the form of a pro-L-selectin after removal of the N-terminal signal peptide, which directs the protein to its cell membrane location. L-selectin is then formed after removal of the N-terminal propeptide. Further, a plurality of N-linked glycosylations occur. Likewise, CD162 is for example synthesized in the form of a pro-protein after removal of the N-terminal signal peptide. CD162 has complex, core-2, sialylated and fucosylated O-linked oligosaccharides and contains the Sialyl-Lewis^{x} (sLe^{x}) glycan. Further, CD162 is postranslationally modified by sulfation, which is required for P- and L-selectin binding. Any of these synthesis steps may be detected alone or in combination, for example based on the accumulation of products of a post-translational modification.

Any method that can be used to detect the presence of a nucleic acid or a protein in the context of the present invention. Such a method may include established standard procedures well known in the art. Examples of such techniques include, but are not limited to, RT-PCR, RNAse protection assay, Northern analysis, Western analysis, ELISA, radioimmunoassay or fluorescence titration assay. The detection method may include an amplification of the signal caused by the nucleic acid or protein, such as a polymerase chain reaction (PCR) or the use of the biotin-streptavidin system, for example in form of a conjugation to an immunoglobulin. The detection method may for example include the use of an antibody, which may be linked to an attached label, such as for instance in Western analysis or ELISA. Where desired, an intracellular antibody may be used for detection. Some or all of the steps of detection may be part of an automated detection system. Illustrative examples of such a system are automated real-time PCR platforms, automated nucleic acid isolation platforms, PCR product analyzers and real-time detection systems. The term “antibody” as used herein, is understood to include an immunoglobulin and an immunoglobulin fragment that is capable of specifically binding a selected protein, e.g. L-selectin or a protein specific for T cells, as well as a respective proteinaceous binding molecule with immunoglobulin-like functions. An antibody may for instance be an EGF-like domain, a Kringle-domain, a fibronectin type I domain, a fibronectin type II domain, a fibronectin type III domain, a PAN domain, a G1a domain, a SRCR domain, a Kunitz/Bovine pancreatic trypsin Inhibitor domain, tendamistat, a Kazal-type serine protease inhibitor domain, a Trefoil (P-type) domain, a von Willebrand factor type C domain, an Anaphylatoxin-like domain, a CUB domain, a thyroglobulin type I repeat, an LDL-receptor class A domain, a Sushi domain, a Link domain, a Thrombospondin type I domain, an immunoglobulin domain or a an immunoglobulin-like domain (for example a domain antibody or a camel heavy chain antibody), a C-type lectin domain, a MAM domain, a von Willebrand factor type A domain, a Somatomedin B domain, a WAP-type four disulfide core domain, a F5/8 type C domain, a Hemopexin domain, an SH2 domain, an SH3 domain, a Laminin-type EGF-like domain, a C2 domain, a “Kappabody” (Ill. et al., Protein Eng (1997) 10, 949-957), a “Minibody” (Martin et al., EMBO J (1994) 13, 5303-5309), a “Diabody” (Holliger et al., PNAS U.S.A. 90, 6444-6448 (1993)), a “Janusin” (Traunecker et al., EMBO J (1991) 10, 3655-3659 or Traunecker et al., Int J Cancer (1992) Suppl 7, 51-52), a nanobody, an adnectin, a tetranectin, a microbody, an affilin, an affibody or an ankyrin, a crystallin, a knottin, ubiquitin, a zinc-finger protein, an autofluorescent protein, an ankyrin or ankyrin repeat protein or a leucine-rich repeat protein (cf. also below).

A measurement of a level or amount may for instance rely on spectroscopic, photochemical, photometric, fluorometric, radiological, enzymatic or thermodynamic means. An example of a spectroscopical detection method is fluorescence correlation spectroscopy. A photochemical method is for instance photochemical cross-linking. The use of photoactive, fluorescent, radioactive or enzymatic labels, respectively are examples for photometric, fluorometric, radiological and enzymatic detection methods. An example of a thermodynamic detection method is isothermal titration calorimetry. The measurement used is generally selected to be of a sensitivity that allows detection of CD62L or LFA-1 expressing cells in the range of a selected threshold value, in particular of a sensitivity that allows determining whether CD62L or LFA-1 expressing cells are above or below the threshold value. The term “determining” generally refer to any form of measurement, and include determining if an element is present or absent. These terms may include either quantitative and/or qualitative determinations. Typically a binding partner of CD62L and LFA-1, respectively, may be used in combination with a detectable marker. Such a binding partner of CD62L or LFA-1 has a detectable affinity and specificity for CD62L or LFA-1, respectively. Typically, binding is considered specific when the binding affinity is higher than 10⁻⁶ M. A binding partner of CD62L and LFA-1, respectively, has in some embodiments an affinity of about 10⁻⁸ M or higher, or of about 10⁻⁹ M or higher. As indicated above, in some embodiments T cells in the sample are identified by the presence of the CD3 protein on their surface; or T cells may be enriched or isolated via the the presence of the CD3 protein on their surface. Identification of CD3⁺ T cells may again be carried out using spectroscopic, photochemical, photometric, fluorometric, radiological, enzymatic or thermodynamic means. Identification and enrichment or isolation of T cells may likewise be carried out by using a suitable binding partner of CD3⁺. Accordingly the above said applies mutatis mutandis to identifying and enriching or isolating T cells. Further, T cells may be identified or isolated in a similar manner, using suitable surface proteins known in the art, for example the T cell receptor. In some embodiments a suitable binding partner of CD3 and a further suitable binding partner of a surface protein characteristic for T cells such as the T cell receptor are combined to identify CD3⁺ T cells. Typically a binding partner of CD3 may be used in combination with a detectable marker. Likewise a binding partner of CD3 may be used in combination with a detectable marker. In some embodiments a suitable binding partner of CD3, a suitable binding partner of a surface protein characteristic for T cells such as the T cell receptor and a suitable binding partner of CD62L are combined to identify CD62L expressing CD3⁺ T cells. In some embodiments a suitable binding partner of CD3⁺, a suitable binding partner of a surface protein characteristic for T cells such as the T cell receptor and a suitable binding partner of LFA-1 are combined to identify LFA-1 expressing CD3⁺ T cells. In some embodiments a suitable binding partner of CD3 and a suitable binding partner of CD62L are combined to identify CD62L expressing T cells. In some embodiments a suitable binding partner of CD3⁻ and a suitable binding partner of LFA-1 are combined to identify LFA-1 expressing T cells.

The term “specific” as used herein is understood to indicate that the binding partner is directed against, binds to, or reacts with CD62L and CD3, respectively. Thus, being directed to, binding to or reacting with includes that the binding partner specifically binds to CD62L, LFA-1, CD4, CD8 or CD3, as applicable. The term “specifically” in this context means that the binding partner reacts with CD62L, LFA-1, CD4, CD8 or CD3, as applicable, or/and a portion thereof, but at least essentially not with another protein. The term “another protein” includes any protein, including proteins closely related to or being homologous to e.g. CD62L, LFA-1 or CD3 against which the binding partner is directed to. The term “does not essentially bind” means that the binding partner does not have particular affinity to another protein, i.e., shows a cross-reactivity of less than about 30%, when compared to the affinity to CD62L, LFA-1 or CD3. In some embodiments the binding partner shows a cross-reactivity of less than about 20%, such as less than about 10%. In some embodiments the binding partner shows a cross-reactivity of less than about 9, 8, or 7%, when compared to the affinity to CD62L, LFA-1 or CD3. In some embodiments the binding partner shows a cross-reactivity of less than about 6%, such as less than about 5%, when compared to the affinity to CD62L, LFA-1 or CD3. Whether the binding partner specifically reacts as defined herein above can easily be tested, inter alia, by comparing the reaction of a respective binding partner with CD62L, with LFA-1 or with CD3, as applicable, and the reaction of the binding partner with (an) other protein(s). The term “specifically recognizing”, which can be used interchangeably with the terms “directed to” or “reacting with” means in the context of the present disclosure that a particular molecule, generally an immunoglobulin, an immunoglobulin fragment or a proteinaceous binding molecule with immunoglobulin-like functions is capable of specifically interacting with and/or binding to at least two, including at least three, such as at least four or even more amino acids of an epitope as defined herein. Generally the immunoglobulin or proteinaceous binding molecule can thereby form a complex with the respective epitope of e.g. CD62L, LFA-1 or CD3. Such binding may be exemplified by the specificity of a “lock-and-key-principle”. “Specific binding” can also be determined, for example, in accordance with Western blots, ELISA-, RIA-, ECL-, IRMA-tests, FACS, IHC and peptide scans.

A respective binding partner of e.g. CD62L, LFA-1 or CD3, as well as a binding partner for another selected cell-characteristic protein, may be an immunoglobulin, a fragment thereof or a proteinaceous binding molecule with immunoglobulin-like functions. Examples of (recombinant) antibody fragments are immunoglobulin fragments such as Fab fragments, Fv fragments, single-chain Fv fragments (scFv), diabodies or domain antibodies (Holt, L. J., et al., Trends Biotechnol. (2003), 21, 11, 484-490). An example of a proteinaceous binding molecule with antibody-like functions is a mutein based on a polypeptide of the lipocalin family (WO 03/029462, Beste et al., Proc. Natl. Acad. Sci. USA (1999) 96, 1898-1903). Lipocalins, such as the bilin binding protein, the human neutrophil gelatinase-associated lipocalin, human Apolipoprotein D or glycodelin, possess natural ligand-binding sites that can be modified so that they bind to selected small protein regions known as haptens. Examples of other proteinaceous binding molecules are the so-called glubodies (see e.g. international patent application WO 96/23879 or Napolitano, E. W., et al., Chemistry & Biology (1996) 3, 5, 359-367), proteins based on the ankyrin scaffold (Mosavi, L. K., et al., Protein Science (2004) 13, 6, 1435-1448) or crystalline scaffold (e.g. internation patent application WO 01/04144) the proteins described in Skerra, J. Mol. Recognit. (2000) 13, 167-187, AdNectins, tetranectins and avimers. Avimers contain so called A-domains that occur as strings of multiple domains in several cell surface receptors (Silverman, J., et al., Nature Biotechnology (2005) 23, 1556-1561). Adnectins, derived from a domain of human fibronectin, contain three loops that can be engineered for immunoglobulin-like binding to targets (Gill, D. S. & Damle, N. K., Current Opinion in Biotechnology (2006) 17, 653-658). Tetranectins, derived from the respective human homotrimeric protein, likewise contain loop regions in a C-type lectin domain that can be engineered for desired binding (ibid.). Peptoids, which can act as protein ligands, are oligo(N-alkyl) glycines that differ from peptides in that the side chain is connected to the amide nitrogen rather than the a carbon atom. Peptoids are typically resistant to proteases and other modifying enzymes and can have a much higher cell permeability than peptides (see e.g. Kwon, Y.-U., and Kodadek, T., J. Am. Soc. (2007) 129, 1508-1509). A molecule that forms a complex with a binding partner of e.g. CD62L, LFA-1 or CD4 may likewise be an immunoglobulin, a fragment thereof or a proteinaceous binding molecule with immunoglobulin-like functions, as explained above. Thus, in an exemplary embodiment detecting the amount of CD62L, e.g. on a cell surface, may carried out using a first antibody or antibody fragment capable of specifically binding CD62L, as well as a second antibody or antibody fragment capable of specifically binding the first antibody or antibody fragment.

An immunoglobulin may be monoclonal or polyclonal. The term “polyclonal” refers to immunoglobulins that are heterogenous populations of immunoglobulin molecules derived from the sera of animals immunized with an antigen or an antigenic functional derivative thereof. For the production of polyclonal immunoglobulins, one or more of various host animals may be immunized by injection with the antigen. Various adjuvants may be used to increase the immunological response, depending on the host species. “Monoclonal immunoglobulins” or “Monoclonal antibodies” are substantially homogenous populations of immunoglobulins to a particular antigen. They may be obtained by any technique which provides for the production of immunoglobulin molecules by continuous cell lines in culture. Monoclonal immuno-globulins may be obtained by methods well known to those skilled in the art (see for example, Köhler et al., Nature (1975) 256, 495-497, and U.S. Pat. No. 4,376,110). An immunoglobulin or immunoglobulin fragment with specific binding affinity only for e.g. CD62L, CD3, LFA-1, CD8 or CD4 can be isolated, enriched, or purified from a prokaryotic or eukaryotic organism. Routine methods known to those skilled in the art enable production of both immunoglobulins or immunoglobulin fragments and proteinaceous binding molecules with immunoglobulin-like functions, in both prokaryotic and eukaryotic organisms.

In this regard the terms “immunize”, “immunization”, or “immunizing” refer to exposing the immune system of an animal to an antigen or to an epitope thereof as illustrated in more detail below. The antigen may be introduced into the animal using a desired route of administration, such as injection, inhalation or ingestion. Upon a second exposure to the same antigen, the adaptive immune response, in particular T cell and B cell responses, is enhanced.

In more detail, an immunoglobulin may be isolated by comparing its binding affinity to a protein of interest, e.g. L-selectin, with its binding affinity to other polypeptides. Humanized forms of the antibodies of the present invention may be generated using one of the procedures known in the art such as chimerization or CDR grafting. In general, techniques for preparing monoclonal antibodies and hybridomas are well known in the art. Any animal such as a goat, a mouse or a rabbit that is known to produce antibodies can be immunized with the selected polypeptide, e.g. L-selectin. Methods for immunization are well known in the art. Such methods include subcutaneous or intraperitoneal injection of the polypeptide. One skilled in the art will recognize that the amount of polypeptide used for immunization and the immunization regimen will vary based on the animal which is immunized, including the species of mammal immunized, its immune status and the body weight of the mammal, as well as the antigenicity of the polypeptide and the site of injection.

The polypeptide may be modified or administered in an adjuvant in order to increase the peptide antigenicity. Methods of increasing the antigenicity of a polypeptide are well known in the art. Such procedures include coupling the antigen with a heterologous protein (such as globulin or β-galactosidase) or through the inclusion of an adjuvant during immunization.

Typically, the immunized mammals are bled and the serum from each blood sample is assayed for particular antibodies using appropriate screening assays. As an illustrative example, anti-CD62L or anti-LFA-1 immunoglobulins may be identified by immunoprecipitation of 125I-labeled cell lysates from CD62L or LFA-1-expressing cells (see, Sanchez-Madrid et al., 1986 and Hemler et al., 1987). Anti-CD62L or anti-LFA-1 immunoglobulins may also be identified by flow cytometry, e.g., by measuring fluorescent staining of Ramos cells incubated with an antibody believed to recognize CD62L or LFA-1.

For monoclonal immunoglobulins, lymphocytes, typically splenocytes, from the immunized animals are removed, fused with an immortal cell line, typically myeloma cells, such as SP2/0-Agl4 myeloma cells, and allowed to become monoclonal immunoglobulin producing hybridoma cells. Typically, the immortal cell line such as a myeloma cell line is derived from the same mammalian species as the lymphocytes. Illustrative immortal cell lines are mouse myeloma cell lines that are sensitive to culture medium containing hypoxanthine, aminopterin and thymidine (“HAT medium”). Typically, HAT-sensitive mouse myeloma cells are fused to mouse splenocytes using 1500 molecular weight polyethylene glycol (“PEG 1500”). Hybridoma cells resulting from the fusion may then be selected using HAT medium, which kills unfused and unproductively fused myeloma cells (unfused splenocytes die after several days because they are not transformed).

Any one of a number of methods well known in the art can be used to identify a hybridoma cell which produces an immunoglobulin with the desired characteristics. Typically the culture supernatants of the hybridoma cells are screened for immunoglobulins against the antigen. Suitable methods include, but are not limited to, screening the hybridomas with an ELISA assay, Western blot analysis, or radioimmunoassay (Lutz et al., Exp. Cell Res. [1988] 175, 109-124). Hybridomas prepared to produce anti-CD62L or anti-LFA-1 immunoglobulins may for instance be screened by testing the hybridoma culture supernatant for secreted antibodies having the ability to bind to a recombinant CD62L or LFA-1 expressing cell line. To produce antibody homologs which are within the scope of the invention, including for example, anti-CD62L or anti-LFA-1 antibody homologs, that are intact immunoglobulins, hybridoma cells that tested positive in such screening assays can be cultured in a nutrient medium under conditions and for a time sufficient to allow the hybridoma cells to secrete the monoclonal immunoglobulins into the culture medium. Tissue culture techniques and culture media suitable for hybridoma cells are well known in the art. The conditioned hybridoma culture supernatant may be collected and for instance the anti-CD62L immunoglobulins optionally further purified by well-known methods. Alternatively, the desired immunoglobulins may be produced by injecting the hybridoma cells into the peritoneal cavity of an unimmunized mouse. The hybridoma cells proliferate in the peritoneal cavity, secreting the immunoglobulin which accumulates as ascites fluid. The immunoglobulin may be harvested by withdrawing the ascites fluid from the peritoneal cavity with a syringe.

Hybridomas secreting the desired immunoglobulins are cloned and the class and subclass are determined using procedures known in the art. For polyclonal immunoglobulins, immunoglobulin containing antisera is isolated from the immunized animal and is screened for the presence of immunoglobulins with the desired specificity using one of the above-described procedures. The above-described antibodies may also be immobilized on a solid support. Examples of such solid supports include plastics such as polycarbonate, complex carbohydrates such as agarose and sepharose, acrylic resins and such as polyacrylamide and latex beads. Techniques for coupling antibodies to such solid supports are well known in the art.

A plurality of conventional display technologies is available to select an immunoglobulin, immunoglobulin fragment or proteinaceous binding molecule. Li et al. (Organic & Biomolecular Chemistry (2006), 4, 3420-3426) have for example demonstrated how a single-chain Fv fragment capable of forming a complex with a selected DNA adapter can be obtained using phage display. Display techniques for instance allow the generation of engineered immunoglobulins and ligands with high affinities for a selected target molecule. It is thus also possible to display an array of peptides or proteins that differ only slightly, typically by way of genetic engineering. Thereby it is possible to screen and subsequently evolve proteins or peptides in terms of properties of interaction and biophysical parameters. Iterative rounds of mutation and selection can be applied on an in vitro basis.

In vitro display technology for the selection of peptides and proteins relies on a physical linkage between the peptide or protein and a nucleic acid encoding the same. A large panel of techniques has been established for this purpose, with the most commonly used being phage/virus display, ribosome display, cell-surface display, ‘peptides on plasmids’, mRNA display, DNA display, and in vitro compartmentalisation including micro-bead display (for reviews see e.g. Rothe, A., et al., FASEB J. (2006) 20, 1599-1610; Sergeeva, A., et al., Advanced Drug Delivery Reviews (2006) 58, 1622-1654).

Different means of physically linking a protein or peptide and a nucleic acid are also available. Expression in a cell with a cell surface molecule, expression as a fusion polypeptide with a viral/phage coat protein, a stabilised in vitro complex of an RNA molecule, the ribosome and the respective polypeptide, covalent coupling in vitro via a puromycin molecule or via micro-beads are examples of ways of linking the protein/peptide and the nucleic acid presently used in the art. A further display technique relies on a water-in-oil emulsion. The water droplets serve as compartments in each of which a single gene is transcribed and translated (Tawfik, D. S., & Griffiths, A. D., Nature Biotech. (1998) 16, 652-656, US patent application 2007/0105117). This physical linkage between the peptide or protein and the nucleic acid (encoding it) provides the possibility of recovering the nucleic acid encoding the selected protein or peptide. Compared to techniques such as immunoprecipitation, in display techniques thus not only binding partners of a selected target molecule can be identified or selected, but the nucleic acid of this binding partner can be recovered and used for further processing. Present display techniques thus provide means for e.g. target discovery, lead discovery and lead optimisation. Vast libraries of peptides or proteins, e.g. antibodies, potentially can be screened on a large scale.

As indicated above, a detectable marker may be coupled to a binding partner of CD62L, of LFA-1, of CD4, of CD8 or CD3, as the case may be, or a molecule that forms a complex with the binding partner of CD62L, LFA-1, CD4, CD8 or CD3. A respective detectable marker, which may be coupled to a binding partner of CD62L, LFA-1, CD4, CD8 or CD3, or a molecule that forms a complex therewith, may be an optically detectable label, a fluorophore, or a chromophore. Examples of suitable labels include, but are not limited to, an organic molecule, an enzyme, a radioactive, fluorescent, and/or chromogenic moiety, a luminescent moiety, a hapten, digoxigenin, biotin, a metal complex, a metal and colloidal gold. Accordingly an excitable fluorescent dye, a radioactive amino acid, a fluorescent protein or an enzyme may for instance be used to detect e.g. the level of CD62L. Examples of suitable fluorescent dyes include, but are not limited to, fluorescein isothiocyanate, 5,6-carboxymethyl fluorescein, Cascade Blue®, Oregon Green®, Texas red, nitrobenz-2-oxa-1,3-diazol-4-yl, coumarin, dansyl chloride, rhodamine, amino-methyl coumarin, DAPI, Eosin, Erythrosin, BODIPY®, pyrene, lissamine, xanthene, acridine, an oxazine, phycoerythrin, a Cy dye such as Cy3, Cy3.5, Cy5, Cy5PE, Cy5.5, Cy7, Cy7PE or Cy7APC, an Alexa dye such as Alexa 647, and NBD (Naphthol basic dye). Examples of suitable fluorescent protein include, but are not limited to, EGFP, emerald, EYFP, a phycobiliprotein such as phycoerythrin (PE) or allophycocyanin, Monomeric Red Fluorescent Protein (mRFP), mOrange, mPlum and mCherry. In some embodiments a reversibly photoswitchable fluorescent protein such as Dronpa, bsDronpa and Padron may be employed (Andresen, M., et al., Nature Biotechnology (2008) 26, 9, 1035). Regarding suitable enzymes, alkaline phosphatase, soybean peroxidase, or horseradish peroxidase may serve as a few illustrative examples. In some embodiments a method of detection may include electrophoresis, HPLC, flow cytometry, fluorescence correlation spectroscopy or a modified form of these techniques. Some or all of these steps may be part of an automated separation/detection system.

In some embodiments the binding partner of e.g. CD62L, LFA-1 or CD3, as well as a binding partner for another selected cell-characteristic protein, further includes a capture molecule. Such a capture molecule allows immobilization of the binding partner, and thereby also of a complex formed between e.g. CD62L, LFA-1 or CD3, or another selected cell-characteristic protein, on a surface or on a polymeric molecule, including an immunoglobulin, an immunoglobulin fragment or a proteinaceous binding molecule with antibody-like functions. A respective surface may for instance be the surface of a micro- or nanoparticle, the surface of a container or the surface of a particularly designed device used for presentation purposes during measurement. A micro- or nanoparticle may in some embodiments include, essentially consist of or consist of a metal, a metalloid or a polymer. In some embodiments the micro- or nanoparticle is magnetic, such as paramagnetic or supermagnetic. The capture molecule may be immobilised on the surface via a covalent bond or a non-covalent bond.

The capture molecule has an affinity to a binding partner of the capture molecule and is capable of forming a complex with the binding partner of the capture molecule. Hence, the capture molecule and the binding partner of the capture molecule define a specific binding pair. Accordingly, a pair of capture molecule and binding partner of the capture molecule may be selected as desired, for example according to the binding partner of CD62L, LFA-1 or CD3 or to the measurement conditions used in detection of for instance CD62L. Examples of a capture molecule include, but are not limited to, a nucleic acid molecule, an oligonucleotide, a protein, an oligopeptide, a polysaccharide, an oligosaccharide, a synthetic polymer, a drug candidate molecule, a drug molecule, a drug metabolite, a metal ion, and a vitamin. Three illustrative examples of suitable capture molecule are biotin, dinitrophenol or digoxigenin. Where the binding partner of the capture molecule is a protein, a polypeptide, or a peptide, further examples of a capture molecule include, but are not limited to, a streptavidin binding tag such as the STREP-TAGS® described in US patent application US 2003/0083474, U.S. Pat. Nos. 5,506,121 or 6,103,493, an immunoglobulin domain, maltose-binding protein, glutathione-S-transferase (GST), calmodulin binding peptide (CBP), FLAG-peptide (e.g. of the sequence Asp-Tyr-Lys-Asp-Asp-Asp-Asp-Lys-Gly), the T7 epitope (Ala-Ser-Met-Thr-Gly-Gly-Gln-Gln-Met-Gly), maltose binding protein (MBP), the HSV epitope of the sequence Gln-Pro-Glu-Leu-Ala-Pro-Glu-Asp-Pro-Glu-Asp of herpes simplex virus glycoprotein D, the Vesicular Stomatitis Virus Glycoprotein (VSV-G) epitope of the sequence Tyr-Thr-Asp-Ile-Glu-Met-Asn-Arg-Leu-Gly-Lys, the hemagglutinin (HA) epitope of the sequence Tyr-Pro-Tyr-Asp-Val-Pro-Asp-Tyr-Ala and the “myc” epitope of the transcription factor c-myc of the sequence Glu-Gln-Lys-Leu-Ile-Ser-Glu-Glu-Asp-Leu. Where the binding partner of the capture molecule is a nucleic acid, a polynucleotide or an oligonucleotide, a capture molecule may furthermore be an oligonucleotide. Such an oligonucleotide tag may for instance be used to hybridize to an immobilised oligonucleotide with a complementary sequence.

As an illustrative example, the capture molecule may be a metal ion bound by a respective metal chelator, such as ethylenediamine, ethylenediaminetetraacetic acid (EDTA), ethylene glycol tetraacetic acid (EGTA), diethylenetriaminepentaacetic acid (DTPA), N,N-bis(carboxymethyl)glycine (also called nitrilotriacetic acid, NTA), 1,2-bis(o-aminophenoxy)ethane-N,N,N′,N′-tetraacetic acid (BAPTA), 2,3-dimercapto-1-propanol (dimmercaprol), porphine or heme. A respective metal ion may define a receptor molecule for a peptide of a defined sequence, which may also be included in a protein. In line with the standard method of immobilised metal affinity chromatography used in the art, for example an oligohistidine tag of a respective peptide or protein is capable of forming a complex with copper (Cu²⁺), nickel (Ni²⁻), cobalt (Co²⁺), or zink (Zn²⁺) ions, which can for instance be presented by means of the chelator nitrilotriacetic acid (NTA).

The capture molecule may be immobilised on a surface (vide infra) such as the surface of a particle such as a metal containing bead. The capture molecule may be immobilised by any means. It may be immobilised on a portion or the entire area of a surface. An illustrative example is the mechanical spotting of a nucleic acid capture molecule onto a metal surface. This spotting may be carried out manually, e.g. by means of a pipette, or automatically, e.g. by means of a micro robot. As an illustrative example, a protein capture molecule, a peptide capture molecule or the polypeptide backbone of a PNA capture molecule may be covalently linked to a gold surface via a thio-ether-bond.

In embodiments where both the capture molecule and the binding partner of the capture molecule are a nucleic acid molecule, including an oligonucleotide, the capture molecule typically has a nucleotide sequence that is at least partially complementary to a portion of a strand of the binding partner of the capture molecule. As a further illustrative example, Avidin or streptavidin may be employed to immobilise a biotinylated nucleic acid, or a biotin containing monolayer of gold may be employed (Shumaker-Parry, J. S., et al., Anal. Chem. (2004) 76, 918). As another illustrative example, the capture molecule may be a metal ion bound by a respective metal chelator (see above).

In this regard the term “capture probe” as used herein refers to matter, such as a molecule, in particular a polymeric molecule, that can bind a nucleic acid molecule such as a DNA or an RNA molecule, including an mRNA molecule, as well as a peptide, a protein, a saccharide, a polysaccharide or a lipid through an interaction that is sufficient to permit the agent to form a complex with the nucleic acid molecule, peptide, protein or saccharide, a polysaccharide or a lipid, generally via non-covalent bonding. In some embodiments the capture probe is a PNA molecule. As indicated above, a PNA molecule is a nucleic acid molecule in which the backbone is a pseudopeptide rather than a sugar. Accordingly, PNA generally has a charge neutral backbone, in contrast to DNA or RNA. Nevertheless, PNA is capable of hybridising at least complementary and substantially complementary nucleic acid strands, just as e.g. DNA or RNA (to which PNA is considered a structural mimic). In some embodiments the capture probe is an aptamer, including a Spiegelmer®, described in e.g. WO 01/92655. An aptamer is typically a nucleic acid molecule that can be selected from a random nucleic acid pool based on its ability to bind a selected other molecule such as a peptide, a protein, a nucleic acid molecule a or a cell. Aptamers, including Spiegelmers, are able to bind molecules such as peptides, proteins and low molecular weight compounds. Spiegelmers® are composed of L-isomers of natural oligonucleotides. Aptamers are engineered through repeated rounds of in vitro selection or through the SELEX (systematic evolution of ligands by exponential enrichment) technology. The affinity of Spiegelmers to their target molecules often lies in the pico- to nanomolar range and is thus comparable to immunoglobulins. An aptamer may also be a peptide. A peptide aptamer consists of a short variable peptide domain, attached at both ends to a protein scaffold.

In typical embodiments the capture probe is an immunoglobulin or of a proteinaceous binding molecule with immunoglobulin-like functions as defined above. In some embodiments the capture probe may be detectably labelled as explained above, for example where the capture probe is intended to be used together with a detection agent that binds to the biomarker and/or the capture probe. The capture probe and/or a respective detection agent may be detectably labelled by linking the same, typically covalently, to a detectable marker such as a radioactive label, a fluorescent moiety, a chemical entity of low molecular weight, an oligonucleotide, an enzyme, or a protein such as a fluorescent protein such as a Green Fluorescent Protein (cf. above). It is understood that the method may also include any molecules which can be used to indirectly indicate the level of the target molecule of interest such as CD62L, CD3, CD4, CD8, CD18 or CD11a. The capture probe may in some embodiments be an immunoglobulin, a portion thereof, a proteinaceous binding molecule with immunoglobulin-like functions, a receptor for the biomarker or a portion thereof or a ligand for the biomarker or a portion thereof. The detection agent may in some embodiments be an immunoglobulin, a portion thereof, a proteinaceous binding molecule with immunoglobulin-like functions, a receptor for the biomarker or a portion thereof, a ligand for the biomarker or a portion thereof or a capture probe or a portion thereof.

In some embodiments a capture probe capable of binding a particular target nucleic acid molecule such as an mRNA molecule encoding e.g. CD62L, CD18 or CD11a, is a nucleic acid molecule that includes a nucleotide sequence that is at least partially complementary to a portion of a strand of such a target nucleic acid molecule. A nucleotide sequence is the complement of another nucleotide sequence if all of the nucleotides of the first sequence are complementary to all of the nucleotides of the second sequence. Accordingly, the respective nucleotide sequence will specifically hybridise to, or undergo duplex formation with, the respective portion of the target nucleic acid molecule under suitable hybridisation assay conditions, in particular of ionic strength and temperature.

As an illustrative example, a single-stranded nucleic acid molecule may be selected as a nucleic acid capture probe. Such a single-stranded nucleic acid molecule may have a nucleic acid sequence that is at least partially complementary to at least a portion of a strand of the target nucleic acid molecule. The respective nucleotide sequence of the nucleic acid capture molecule may for example be 70, for example 80 or 85, including 100% identical to another nucleic acid sequence. The higher the percentage to which the two sequences are complementary to each other (i.e. the lower the number of mismatches), the higher is typically the sensitivity of the method of the invention. In typical embodiments the respective nucleotide sequence is substantially complementary to at least a portion of the target nucleic acid molecule. “Substantially complementary” as used herein refers to the fact that a given nucleic acid sequence is at least 90% identical to another nucleic acid sequence. A substantially complementary nucleic acid sequence is in some embodiments 95%, such as 100% identical to another nucleic acid sequence. The term “complementary” or “complement” refers to two nucleotides that can form multiple favourable interactions with one another. Such favourable interactions are specific association between opposing or adjacent pairs of nucleic acid (including nucleic acid analogue) strands via matched bases, and include Watson-Crick base pairing. As an illustrative example, in two given nucleic acid molecules (e.g. DNA molecules) the base adenosine is complementary to thymine or uracil, while the base cytosine is complementary to guanine. A nucleic acid probe used in the context of the present invention may be used to probe the sample by usual hybridization methods to detect the presence of nucleic acid molecules encoding e.g. CD62L, CD18 or CD11a.

The term “nucleic acid molecule” as used herein refers to any nucleic acid in any possible configuration, such as single stranded, double stranded or a combination thereof. Examples of nucleic acids include for instance DNA molecules, RNA molecules, analogues of the DNA or RNA generated using nucleotide analogues or using nucleic acid chemistry, locked nucleic acid molecules (LNA), protein nucleic acids molecules (PNA), alkylphosphonate and alkylphosphotriester nucleic acid molecules and tecto-RNA molecules (e.g. Liu, B., et al., J. Am. Chem. Soc. (2004) 126, 4076-4077). LNA has a modified RNA backbone with a methylene bridge between C4′ and O2′, providing the respective molecule with a higher duplex stability and nuclease resistance. Alkylphosphonate and alkylphosphotriester nucleic acid molecules can be viewed as a DNA or an RNA molecule, in which phosphate groups of the nucleic acid backbone are neutralized by exchanging the P—OH groups of the phosphate groups in the nucleic acid backbone to an alkyl and to an alkoxy group, respectively. DNA or RNA may be of genomic or synthetic origin and may be single or double stranded. Such nucleic acid can be e.g. mRNA, cRNA, synthetic RNA, genomic DNA, cDNA synthetic DNA, a copolymer of DNA and RNA, oligonucleotides, etc. A respective nucleic acid may furthermore contain non-natural nucleotide analogues and/or be linked to an affinity tag or a label.

Many nucleotide analogues are known and can be used in nucleic acids used in the methods of the invention. A nucleotide analogue is a nucleotide containing a modification at for instance the base, sugar, or phosphate moieties. As an illustrative example, a substitution of 2′-OH residues of siRNA with 2′F, 2′O-Me or 2′H residues is known to improve the in vivo stability of the respective RNA. Modifications at the base moiety may be a natural or a synthetic modification of A, C, G, and T/U, a different purine or pyrimidine base, such as uracil-5-yl, hypoxanthin-9-yl, and 2-aminoadenin-9-yl, as well as a non-purine or a non-pyrimidine nucleotide base. Other nucleotide analogues serve as universal bases. Examples of universal bases include 3-nitropyrrole and 5-nitroindole. Universal bases are able to form a base pair with any other base. Base modifications often can be combined with for example a sugar modification, such as for instance 2′-O-methoxyethyl, e.g. to achieve unique properties such as increased duplex stability.

Interactions between two or more nucleic acid molecules are generally sequence driven interactions referred to as hybridization. Sequence driven interaction is an interaction that occurs between two nucleotides or nucleotide analogs or nucleotide derivatives in a nucleotide specific manner (supra). Typically sequence driven interactions occur on the Watson-Crick face or Hoogsteen face of the respective nucleotide. The hybridization of two nucleic acids is affected by a number of conditions and parameters known to those skilled in the art. For example, the salt concentrations, pH, and temperature of the reaction all affect whether two nucleic acid molecules will hybridize. For example, in some embodiments selective hybridization conditions can be defined as stringent hybridization conditions. For example, stringency of hybridization is controlled by both temperature and salt concentration of either or both of the hybridization and washing steps. For example, conditions of hybridization that achieve selective interactions between complementary sequences may involve hybridization in high ionic strength solution (6×SSC or 6×SSPE) at a temperature that is in the range from about 12 to about 25° C. below the Tm, the melting temperature at which half of the molecules dissociate from their hybridization partners, followed by washing at a combination of temperature and salt concentration chosen so that the washing temperature is in the range from about 5° C. to about 20° C. below the Tm. The temperature and salt conditions are readily determined empirically in preliminary experiments in which samples of reference DNA immobilized on filters are hybridized to a labelled nucleic acid of interest and then washed under conditions of different stringencies. Hybridization temperatures are typically higher for DNA-RNA and RNA-RNA hybridizations than for DNA-DNA hybridizations.

In order to obtain nucleic acid probes having nucleotide sequences which correspond to altered portions of the amino acid sequence of the polypeptide of interest, chemical synthesis can be carried out. The synthesized nucleic acid probes may be first used as primers in a polymerase chain reaction (PCR) carried out in accordance with recognized PCR techniques, essentially according to standard PCR protocols utilizing the appropriate template, in order to obtain the probes that can be used in the context of the present invention.

One skilled in the art will readily be able to design such probes based on a sequence as referred to herein using methods of computer alignment and sequence analysis well known in the art. As explained above, a respective hybridization probe can be labelled by standard labelling techniques using a detectable marker, such as with a radiolabel, enzyme label, fluorescent label, biotin-avidin label, or chemiluminescence (supra). After hybridization, the probes may be visualized using known methods. A nucleic acid probe may be immobilized on a solid support. Examples of such solid supports include, but are not limited to, plastics such as polycarbonate, complex carbohydrates such as agarose and sepharose, and acrylic resins, such as polyacrylamide and latex beads. As an illustrative example one or more nucleic acid probes may be bound to or immobilized on a solid support. The solid support may be a chip, for example a DNA microchip. Techniques for coupling nucleic acid probes to such solid supports are well known in the art.

The most frequently used methods for determining the concentration of nucleic acids include the detection by autoradiography, fluorescence, chemiluminescence or bioluminescence as well as electrochemical and electrical techniques. A further suitable technique is the electrical detection of a target nucleic acid molecule as disclosed in international patent applications WO 2009/041917 and WO 2008/097190, both being incorporated herein by reference in their entirety. A technique for the specific detection of a selected nucleic acid well established in the art is based on the hybridisation between a nucleic acid capture probe and a target nucleic acid. Typically the respective nucleic acid capture probe is immobilised onto a solid support, and subsequently one of the above mentioned detection methods is employed.

As indicated above, an immunoglobulin labeled with a fluorescence dye may for instance be used to optically detect the presence of a certain protein or polypeptide. Nucleic acid intercalating dyes, such as YOYO, JOJO, BOBO, POPO, TOTO, LOLO, SYBR, SYTO, SYTOX, PicoGreen, or Oligreen as available from Molecular Probes, may be used for optical detection.

In some embodiments determining the level of expression of the gene of interest includes determining the level of transcription into mRNA. RNA encoding the protein of interest in the sample, such as CD62L, CD11A, CD18, CD3, CD4 or CD8 may be amplified using any available amplification technique, such as polymerase chain reaction (PCR), including multiplex PCR, nested PCR and amplification refractory mutation specific (ARMS) PCR (also called allele-specific PCR (AS-PCR), rolling circle amplification (RCA), nucleic acid sequence based amplification (NASBA), ligase chain reaction (LCR), QB replicase chain reaction, loop-mediated isothermal amplification (LAMP), transcription mediated amplification (TMA) and strand displacement amplification (SDA), including genome strand displacement amplification (WGSDA), multiple strand displacement amplification (MSDA), and gene specific strand displacement amplification (GS-MSDA). Detection of the obtained amplification products may be performed in numerous ways known in the art. Examples include, but are not limited to, electrophoretic methods such as agarose gel electrophoresis in combination with a staining such as ethidium bromide staining In other embodiments the method of the invention is accompanied by real time detection, such as real time PCR. In these embodiments the time course of the amplification process is monitored. A means of real time detection commonly used in the art involves the addition of a dye before the amplification process. An example of such a dye is the fluorescence dye SYBR Green, which emits a fluorescence signal only when bound to double-stranded nucleic acids.

As explained above, typically a detectable label or marker is used. Such a marker or label may be included in a nucleic acid that includes the sequence to be amplified. A marker may also be included in a primer or a probe. It may also be incorporated into the amplification product in the course of the reaction. In some embodiments such a marker compound, e.g. included in a nucleic acid, is an optically detectable label, a fluorophore, or a chromophore. An illustrative example of a marker compound is 6-carboxyfluorescein (FAM).

As an illustrative example, real-time PCR may be used to determine the level of RNA encoding the protein of interest in the sample, such as CD62L, CD11A, CD18, CD3, CD4 or CD8. Such a PCR procedure is carried out under real time detection, so that the time course of the amplification process is monitored. PCR is characterised by a logarithmic amplification of the target sequences. For the amplification of RNA, a reverse transcriptase-PCR is used. Design of the primers and probes required to detect expression of a biomarker of the invention is within the skill of a practitioner of ordinary skill in the art. In some embodiments RNA from the sample is isolated under RNAse free conditions and then converted to DNA via the use of a reverse transcriptase. Reverse transcription may be performed prior to RT-PCR analysis or simultaneously, within a single reaction vessel. RT-PCR probes are oligonucleotides that have a fluorescent moiety, also called reporter dye, attached to the 5′ end and a quencher moiety coupled to the 3′ end (or vice versa). These probes are typically designed to hybridize to an internal region of a PCR product. In the unhybridized state, the proximity of the fluor and the quench molecules prevents the detection of fluorescent signal from the probe. During PCR amplification, when the polymerase replicates a template on which an RT-PCR probe is bound, the 5′-3′ nuclease activity of the polymerase cleaves the probe. Thereby the fluorescent and quenching moieties are decoupled. Fluorescence increases then in each cycle, in a manner proportional to the amount of probe cleavage. Fluorescence signal emitted from the reaction can be measured or followed over time using equipment which is commercially available using routine and conventional techniques. Quantitation of biomarker RNA in a sample being evaluated may be performed by comparison of the amplification signal to that of one or more standard curves where known quantities of RNA were evaluated in a similar manner. In some embodiments, the difference in biomarker expression is measured as the difference in PCR cycle time to reach a threshold fluorescence, or “dCT.”

In some embodiments the level or amount of CD62L, LFA-1 and/or CD3 on the surface of cells in the sample is determined using a flow cytometry based analysis, typically in combination with immunofluorescence. Immunofluorescence is generally achieved using a binding partner as described above, which is linked to, or includes, a fluorophore as a detectable marker (supra). Flow cytometry is a technique for counting, examining, and sorting microscopic particles such as biological cells suspended in a stream of fluid. It allows a simultaneous multiparametric analysis of the physical and chemical characteristics of single cells flowing through an optical or electronic detection device. An illustrative example of a well established flow cytometry based analysis in the art is fluorescence-activated cell sorting (FACS). FACS allows sorting a heterogeneous mixture of cells into a plurality of containers, one cell at a time, based upon the specific light scattering and fluorescent characteristics of each cell. Thereby FACS allows the sorting of subpopulations of cells of interest and their further use in in vitro and in vivo assays. FACS is often be used in combination with monoclonal antibodies as a reagent to detect cells as having a particular antigen, indicative of an expressed protein (supra).

This technique allows the concurrent fast, objective and quantitative recording of fluorescent signals from individual cells and the physical separation of respective cells according to particular interest. Fluorescent signals used in flow cytometry, for instance when quantifying and/or sorting cells by any marker present on or in the cell, are typically fluorescently-tagged antibody preparations or fluorescently-tagged ligands for binding to antibodies or other antigen-, epitope- or ligand-specific agent, such as with biotin/avidin binding systems or fluorescently-labelled and optionally addressable beads (e.g. LUMINEX® microspheres). Depending of the equipment used, any desired detectable marker or combination of detectable markers can be detected by the optics and/or electronics of a flow cytometer. Current three-laser, “multidimensional”, FACS machines enable up to 14 simultaneous single-cell measurements, such as 2 light scatter detectors and 12 fluorescence plus forward detectors allowing for example the detection of fluorescent surface/intracellular markers. As an illustrative example, the three lasers of a FACS machine may be a krypton laser operating at 407 nm, an argon laser operating at 488 nm, and a dye laser operating at 595 nm.

It is to be understood that expression to be detected is not limited to the biomarkers as such, but also that of proteins or mRNA which regulates the biomarker (such as transcription factors), metabolites of said biomarker, or any subunit thereof, or any molecules whose expression can be correlated with the biomarkers. For example, the detection of LFA-1 expression can also include detecting the protein or mRNA of CD11a and Runx3.

For CD62L, monitoring may be targeted at soluble CD62L in the patient. In this case, the sample to be tested may be any bodily fluids such as serum or CSF.

The FACS technique has been used extensively in relation to antigens expressed on the surface of cells, including cells that remain alive during, and after, FACS. Similarly, the method has been used with intracellular reporter gene systems based on the expression of a detectably labelled gene product by the cell. Accordingly, the technique not only allows detecting the presence of e.g. CD62L, LFA-1, CD4 or CD8 on the cell surface, but also detecting the presence of RNA or DNA within the cell, for example RNA encoding CD62L and CD3 or CD4 (vide infra). Therefore FACS can also be used to determine the amount of nucleic acid formation from the SELL gene, which encodes CD62L, in cells, such as T cells, including CD4⁺ T cells or CD8⁺ T cells, of the sample from the subject.

In some embodiments determining the amount of CD62L, LFA-1, CD3, CD4 and/or CD8 on the surface of cells in the sample is carried out by determining the amount of CD62L, LFA-1, CD4 and/or CD8 that is accessible in the sample. Such a method can be taken to be a method of determining extracellular CD62L, LFA-1, CD3, CD4 and/or CD8 in the sample. In embodiments where cells such as T cells are immobilized on a surface, for example using a capture reagent as detailed above, before determining the amount of e.g. CD62L, LFA-1 and/or CD3, any soluble LFA-1, CD62L and/or CD3 can easily be removed, for example by way of washing. In such embodiments therefore only CD62L, LFA-1 and/or CD3 on the surface of cells is being determined. An illustrative example of a suitable technique in this regard is a radiolabel assay such as a Radioimmunoassay (RIA) or an enzyme-immunoassay such as an Enzyme Linked Immunoabsorbent Assay (ELISA). While a RIA is based on the measurement of radioactivity associated with a complex formed between an immunoglobulin or a proteinaceous binding molecule with immunoglobulin-like functions and an antigen, an ELISA is based on the measurement of an enzymatic reaction associated with a complex formed between an immunoglobulin or a proteinaceous binding molecule with immuneglobulin-like functions and an antigen. Typically a radiolabel assay or an enzyme-immunoassay involves one or more separation steps in which a binding partner of e.g. CD62L, LFA-1 or CD3 that has not formed a complex with CD62L, LFA-1 or CD3 is being removed, thereby leaving only binding partner of CD62L, LFA-1 or CD3 behind, which has formed a complex with CD62L, LFA-1 or CD3. This allows the generation of specific signals originating from the presence of CD62L, LFA-1 or CD3.

An ELISA or RIA test can be competitive for measuring the amount of CD62L, LFA-1, CD3, CD4 and/or CD8, i.e. the amount of antigen. For example, an enzyme labelled antigen is mixed with a test sample containing antigen, which competes for a limited amount of immunoglobulin or a proteinaceous binding molecule with immunoglobulin-like functions. The reacted (bound) antigen is then separated from the free material, and its enzyme activity is estimated by addition of substrate. An alternative method for antigen measurement is the double immunoglobulin/proteinaceous binding molecule sandwich technique. In this modification a solid phase is coated with specific immunoglobulin or a proteinaceous binding molecule with immunoglobulin-like functions. This is then reacted with the sample from the subject that contains the antigen. Then enzyme labelled specific immunoglobulin/proteinaceous binding molecule is added, followed by the enzyme substrate. The ‘antigen’ in the test sample is thereby ‘captured’ and immobilized on to the sensitized solid phase where it can itself then immobilize the enzyme labelled immunoglobulin/proteinaceous binding molecule. This technique is analogous to the immunoradiometric assays.

In an indirect ELISA method, an antigen is immobilized by passive adsorption on to the solid phase. A test serum may then be incubated with the solid phase and any immunoglobulin in the test serum forms a complex with the antigen on the solid phase. Similarly a solution of a proteinaceous binding molecule with immunoglobulin-like functions may be incubated with the solid phase to allow the formation of a complex between the antigen on the solid phase and the proteinaceous binding molecule. After washing to remove unreacted serum components an anti-immunoglobulin immunoglobulin anti-proteinaceous binding molecule immunoglobulin, linked to an enzyme is contacted with the solid phase and incubated. Where the second reagent is selected to be a proteinaceous binding molecule with immunoglobulin-like functions, a respective proteinaceous binding molecule that specifically binds to the proteinaceous binding molecule or the immunoglobulin directed against the antigen is used. A complex of the second proteinaceous binding molecule or immunoglobulin and the first proteinaceous binding molecule or immunoglobulin, bound to the antigen, is formed. Washing again removes unreacted material. In the case of RIA radioactivity signals are being detected. In the case of ELISA the enzyme substrate is added. Its colour change will be a measure of the amount of the immobilized complex involving the antigen, which is proportional to the antibody level in the test sample.

In another embodiment the immunoglobulin or the proteinaceous binding molecule with immunoglobulin-like functions may be immobilized onto a surface, such as the surface of a polymer bead (supra), or coated onto the surface of a device such as a polymer plate or a glass plate. As a result the immune complexes can easily be separated from other components present by simply washing the surface, e.g. the beads or plate. This is the most common method currently used in the art and is referred to as solid phase RIA or ELISA. This embodiment may be particularly useful for determining the amount of CD62L, LFA-1, CD4 and/or CD8 on the surface of cells (cf. also above). On a general basis, in any embodiment of a radiolabel assay or of an enzyme-immunoassay passive adsorption to the solid phase can be used in the first step. Adsorption of other reagents can be prevented by inclusion of wetting agents in all the subsequent washing and incubation steps. It may be advantageous to perform washing to prevent carry-over of reagents from one step to the next.

Various other modifications of ELISA have been used in the art. For example, a system where the second proteinaceous binding molecule or immunoglobulin used in the double antibody sandwich method is from a different species, and this is then reacted with an anti-immunoglobulin enzyme conjugate or an anti-proteinaceous binding molecule enzyme conjugate. This technique comes with the potential advantage that it avoids the labelling of the specific immunoglobulin or proteinaceous binding molecule, which may be in short supply and of low potency. This same technique can be used to assay immunoglobulin or proteinaceous binding molecule where only an impure antigen is available; the specific reactive antigens are selected by the antibody immobilized on the solid phase.

In another example of an ELISA assay for an antigen, a surface, a specific antigen is immobilized on a surface, e.g. a plate used, and the surface is then incubated with a mixture of reference immunoglobulins or proteinaceous binding molecules and a test sample. If there is no antigen in the test sample the reference immunoglobulin or proteinaceous binding molecule becomes fixed to an antigen sensitized surface. If there is antigen in the test solution this combines with the reference immunoglobulin or proteinaceous binding molecule, which cannot then react with the sensitized solid phase. The amount of immunoglobulin/proteinaceous binding molecule attached is then indicated by an enzyme labelled anti-globulin/anti-binding molecule conjugate and enzyme substrate. The amount of inhibition of substrate degradation in the test sample (as compared with the reference system) is proportional to the amount of antigen in the test system.

Yet a further technique that can also be carried out to quantify and thus determine the amount of CD62L, LFA-1, CD3, CD4 and/or CD8 on the surface of cells in the sample is Fluorescence Microscopy, including Ratio Fluorescence Microscopy. Fluorescence microscopy has long been used as a descriptive adjunct to quantitative biochemical techniques in studies of cellular organization and physiology. In the late 1970s, sensitive imaging detectors became commercially available and gave fluorescence microscopy the potential to be a quantitative tool. However, because of the prohibitive cost and sophistication of high-speed image processing computers, quantitative fluorescence microscopy was generally limited to relatively few laboratories with a specific interest in “digital imaging microscopy.” This situation has changed in the past 10 years with the revolution in digital technology. Inexpensive personal computers are now capable of tasks that once required large mainframe computers.

Integrated optical imaging systems are commercially available that are capable of processing an entire assay from the biological preparation to the final data. In parallel, significant improvements have been made in optical elements and imaging hardware. Sensitive fluorescent indicators of a variety of physiologically important properties have been introduced, and new fluorescent reagents are continually being developed for sensitively and specifically characterizing the intracellular distribution of proteins, nucleotides, ions, and lipids.

As quantitative microscopy becomes more widely available, researchers new to fluorescence microscopy should be aware of the factors that may complicate quantification of fluorescence. The amount of fluorescence detected is affected by the properties of illumination sources, the optical and spectroscopic properties of the microscope, and the resolution, sensitivity, and signal-to-noise properties of the detector. Fluorescence emissions are attenuated by the photobleaching that accompanies illumination. At high concentrations of fluorophore, interactions between fluorophore moieties can alter the amount and/or spectrum of fluorescence emissions. For certain fluorophores, fluorescence is also sensitive to the immediate physical environment (i.e., for example, ionic composition) of the fluorophore.

In ratio fluorescence microscopy two fluorescence images are collected and the parameter of interest is quantified as a ratio of the fluorescence in one image to that in the other image. An illustrative example of a ratio fluorescent ion indicator includes, but is not limited to, fluorescein and fura-2, the excitation spectra of which change shape upon binding protons or calcium ions, respectively. In the case of fluorescein, fluorescence excited by 490 run light is efficiently quenched by proton binding, whereas fluorescence excited by 450 nm light is relatively unaffected. Although the quantity of fluorescein fluorescence emitted by a volume when excited with 490 nm light depends on the pH of that volume, it is also affected by other factors, including the concentration of fluorescein in the volume. However, the ratio of fluorescence excited by 90 nm light to that excited by 450 nm depends on pH, but is relatively independent of many variables that affect quantification in single wavelength images: fluorophore concentration, photobleaching, lateral heterogeneity in illumination and detector sensitivity, and differences in optical path length. Spectroscopic variation in illumination and detection is circumvented by calibrating the microscopic system with known pH standards.

Fluorescence ratio images may be collected by sequentially exciting the sample with two different wavelengths of light and sequentially collecting two different images, by exciting the sample with a single wavelength of light and collecting images formed from light of two different emission wavelengths, or by exciting the sample with two wavelengths and collecting emissions of two wavelengths. Ion indicators have been developed for both excitation ratio microscopy (i.e., for example, fura-2 for calcium and fluorescein for pH) and for emission ratio microscopy (i.e., for example, indo-1 for calcium and SNARF for pH).

A further technique suitable for determining the amount of CD62L, LFA-1, CD3, CD4 and/or CD8 on the surface of cells is fluorescence resonance energy transfer (FRET). In FRET an excited fluorescent donor molecule, rather than emitting light, transfers that energy via a dipole-dipole interaction to an acceptor molecule in close proximity. If the acceptor is fluorescent, then the decrease in donor fluorescence due to FRET is accompanied by an increase in acceptor fluorescence (i.e., for example, sensitized emission). The amount of FRET depends strongly on distance, typically decreasing as the sixth power of the distance, so that fluorophores can directly report on phenomena occurring on the scale of a few nanometers, well below the resolution of optical microscopes. Among other purposes, FRET has been used to map distances and study aggregation states, membrane dynamics, or DNA hybridization.

In principle, FRET measurements can provide information about any system the components of which can be manipulated to change the proximity of donors and acceptors on the scale of a few nanometers. In practice, the ability to label a system of interest with appropriate donors and acceptors is constrained by several physical and instrumental factors. In addition to the requirement that donor and acceptor be in close proximity, the donor emission and acceptor absorption spectra should overlap significantly with minimal overlap of the direct excitation spectra of the two fluorophores. Instrumental differences between a fluorescence microscope and a spectrofluorometer, i.e., spatial confinement of the signal, reduced sensitivity, and generally limited wavelength selection, all affect the quality and quantity of information that can be extracted from a FRET experiment using a microscope. The use of FRET in its traditional incarnation as a molecular ruler to measure absolute distances is often not feasible in the fluorescence microscope. Rather, FRET ratio imaging microscopy is often used as an indicator of proximity, subject to some degree of calibration.

The simplest experimental approach is to excite the donor and measure both the direct donor emission “DD” and the sensitized emission “DA” of the acceptor (the first letter represents the species being excited, and the second letter represents the observed emission). The ratio of acceptor-to donor fluorescence, DA/DD, varies between two extremes: no energy transfer and maximal energy transfer. When donor and acceptor are sufficiently distant, no energy transfer occurs and the donor fluorescence (DD) is at its maximum, whereas the sensitized emission is zero. Acceptor fluorescence results only from direct excitation of the acceptor, and DA/DD is at its minimum. The greatest amount of energy transfer occurs when the donor and acceptor are separated by the shortest possible distance, and excited donors lose most of their energy to the acceptor.

Complete quantification of FRET can involve significant calculations, but an estimation of FRET can be obtained easily by measuring the intensity at two fixed time points and taking the ratio of these intensities.

To quantify the relative amount of an acceptor, the acceptor can also be excited directly with the wavelength ideal for acceptor fluorescence, so that “AA” is recorded rather than DA. With AA used as the reference, the ratio DD/AA can also be used as a measure of FRET. Measurement of AA does not generally affect the measurement of DD because acceptor excitation wavelengths are always longer (lower energy) than donor excitation wavelengths, thus avoiding photobleaching of the donor.

Although photobleaching should usually be minimized, it can in some cases actually be exploited to measure FRET. Photobleaching of the donor usually occurs when it is in the excited state: before fluorescence emission occurs there is some probability that photobleaching will remove that fluorophore from the excited state, and also from future excitation emission cycles. When FRET occurs, the donor is removed from the excited state before emission or photobleaching, and the bleach rate decreases because that donor remains available for another cycle of excitation emission. The efficiency of FRET can be determined from the bleach rate of donor fluorescence in the presence of acceptor compared with the bleach rate of the donor in the absence of acceptor. Experimentally, the instantaneous intensity, 1(t), is normalized to the initial intensity 1(0) and the decay of fluorescence intensity is analyzed. A major advantage of the photobleaching method is that it uses only a single excitation wavelength and only a single emission wavelength. The bleach rate of the donor in the absence of acceptor should be measured under experimental conditions identical to those for the donor-acceptor pair, because bleaching rates can vary significantly for different intracellular environments.

If (i) the amount of FRET is relatively small; (ii) the acceptor is not fluorescent; or (iii) rapid photobleaching prevents measurement of static fluorescence intensities, a photobleaching method may provide the only practical measurement of FRET. In particular, the photobleaching method should be useful with the high illumination intensities typical with lasers used for confocal microscopy.

Determining the level or amount of CD62L, LFA-1, CD4, CD8 and/or CD3 in the sample typically involves the formation of signals, e.g. signals generated by a detectable marker (supra) that can be quantified. Quantifying the signals in order to determine the level of e.g. CD62L, CD3 and/or LFA-1 in the sample may be carried out by comparing obtained signals with those of one or more reference measurements. As will be apparent from the above, the word “comparing” as used herein refers to a comparison of parameters or values in terms of absolute amounts/levels that correspond to each other. As an example, a number of cells is compared to a reference number of cells, a concentration is compared to a reference concentration, or a signal intensity obtained from a test sample is compared to the intensity of a corresponding type of signal obtained in a reference sample. A respective reference measurement may be based on the signal generated by a known amount of CD62L, LFA-1 and/or CD3. Such a known amount of CD62L, LFA-1 and/or CD3 may for example be present in a sample with a composition that resembles the sample from the subject, in which the amount of CD62L, LFA-1 and/or CD3 is to be determined. A respective reference sample may be taken to define an external reference sample. In some embodiments of a method of the invention an internal reference sample may in addition or alternatively be used. Such an internal reference sample is a sample obtained from the subject at a previous point of time. The amount of CD62L, LFA-1 and/or CD3 in such a sample may be determined to identify the changes in CD62L, LFA-1 and/or CD3 levels in the subject. In some embodiments the level or amount of CD62L, LFA-1 and CD3, respectively, in the sample may be normalized by a comparison to the level of one or more other proteins, typically cell surface proteins that are known in the art to be stably expressed. In some embodiments a technique of determining the number, amount or ratio of T cells that have e.g. CD62L and/or LFA-1 on their surface includes calibrating the analysis equipment. In embodiments where flow cytometry is used, a standardized blood cell sample may for example be used such as the IMMUNO-TROL® Control Cells commercially available from Beckman Coulter Inc. (Fullerton, Calif., USA, order No. 6607077).

In some embodiments of the method of the invention the amount or level of T cells that have both CD62L and CD3 determined in the sample may be compared to a threshold value. In some embodiments of the method of the invention the amount or level of T cells that have both LFA-1 and CD3 determined in the sample may be compared to a threshold value. In some embodiments the amount of T cells that have both CD62L and CD3 determined in the sample may be compared to a threshold value. In some embodiments the amount of T cells that have CD62L, LFA-1 and CD3 determined in the sample may be compared to a threshold value. In some embodiments the amount or level of T cells that have CD62L, LFA-1 and CD3 determined in the sample may be compared to a threshold value. In some embodiments the ratio of T cells that have CD62L and/or LFA-1 and CD3 to T cells that have only CD3, but not CD62L and/or LFA-1, may be determined in the sample may and compared to a threshold ratio. In some embodiments the ratio of T cells that have both CD62L and CD3 or both LFA-1 and CD3 to all T cells that have CD3 may be determined in the sample may and compared to a threshold ratio. In some embodiments the ratio of T cells that have CD62L, LFA-1 and CD3 determined in the sample may be compared to a threshold value. In some embodiments the amount or level of T cells that have CD62L as well as CD3 determined in the sample may be compared to a threshold value. In some embodiments the ratio of T cells that have CD62L and/or LFA-1 and CD3 to T cells that have only CD3, but not CD62L and/or LFA-1, may be determined in the sample and may compared to a threshold ratio. In some embodiments the ratio of T cells that have CD62L, LFA-1 and CD3 determined in the sample may be compared to a threshold value.

A respective threshold value may in some embodiments be a predetermined threshold value. In some embodiments the threshold value is based on the amount of cells having both CD62L and CD3 in a control sample or both LFA-1 and CD3 in a control sample. As applicable, in some embodiments the threshold value is based on the amount of cells having CD62L, LFA-1 and CD3 in a control sample. In some embodiments the threshold value is a threshold ratio based on the ratio of cells that have both CD62L and/or LFA-1 and CD3 to T cells that have only CD3, but not CD62L and/or not LFA-1, or to all T cells that have CD3 in a control sample. A respective control sample may have any condition that varies from the sample main measurement itself. Such a control sample may be a sample of, include or essentially consist of the corresponding body fluid as the sample from the subject. A control sample may for example be a sample, such as a blood sample, a plasma sample, a serum sample or a cerebrospinal fluid (liquor) sample, of a subject known not to suffer from PML. In some embodiments a respective control sample is from a subject that is age-matched. In some embodiments a respective control sample is from a subject that is known not to have a confounding disease, in some embodiments from a subject known not to have PML, or from a subject known to suffer from MS, as applicable, and in some embodiments from a subject known not to have a disease.

In some embodiments a threshold value is based on a control or reference value obtained concomitantly with the value of the sample from the subject. In some embodiments a respective control or reference value is determined at a different point in time, for example at a point in time earlier than the measurement of the sample from the subject is carried out. It is understood that the terms control and reference may in some embodiments be a range of values.

Population studies may also be used to select a threshold value. Receiver Operating Characteristic (“ROC”) arose from the field of signal detection theory developed during World War II for the analysis of radar images, and ROC analysis is often used to select a threshold able to best distinguish a diseased subpopulation from a nondiseased subpopulation. A false positive in this case occurs when a person tests positive, but actually does not have the disease. A false negative, on the other hand, occurs when the person tests negative, suggesting the person is healthy, when it actually does have the disease. To draw a ROC curve, the true positive rate (TPR) and false positive rate (FPR) are determined as the decision threshold is varied continuously. Since TPR is equivalent with sensitivity and FPR is equal to 1−specificity, the ROC graph is sometimes called the sensitivity vs (1−specificity) plot. A perfect test will have an area under the ROC curve of 1.0; a random test will have an area of 0.5. A threshold is selected to provide an acceptable level of specificity and sensitivity.

In addition to threshold comparisons, other methods for correlating assay results to a patient classification (occurrence or nonoccurrence of disease, likelihood of an outcome, etc.) include decision trees, rule sets, Bayesian methods, and neural network methods. These methods can produce probability values representing the degree to which a subject belongs to one classification out of a plurality of classifications.

The comparison to a threshold value, which may be a predetermined threshold value, can be carried out manually, semi-automatically or in a fully automated manner. In some embodiments the comparison may be computer assisted. A computer assisted comparison may employ values stored in a database as a reference for comparing an obtained value or a determined amount, for example via a computer implemented algorithm. Likewise, the comparison to a reference measurement may be carried out manually, semi-automatically or in a fully automated manner, including in a computer assisted manner.

The level of expression of CD62L and LFA-1 determined in or from a sample of a subject may be expressed in terms of cell numbers, i.e. the number of T cells that are positive for CD62L and for LFA-1. The level of expression of CD62L and LFA-1 may also be expressed in terms of the total amount of CD62L and LFA-1 in a sample. As explained above, where immobilization of cells onto a surface is employed, for example an immobilized capture probe specific for T cells, the total amount of CD62L and LFA-1 present on the respective cells may be used to express the total amount of CD62L and LFA-1. In some embodiments a high level of soluble CD62L can be expected to be included in the sample from a patient. Soluble CD62L originates for example from granulocytes. In such embodiments it may be advantageous to distinguish soluble CD62L and CD62L present on the surface of cells or to remove soluble CD62L before detecting CD62L in the detection method. Whether high levels of soluble CD62L are to be expected in a sample can easily be tested by for instance measuring a single value of the sample with and/without immobilizing T cells and subsequently washing the same. A significant difference of the obtained values indicates a high amount of soluble CD62L in the sample. The term “significant” is used to indicate that the level of decrease or increase is of statistical relevance, and typically means a deviation of a value relative to another value of about 2 fold or more, including 3 fold or more, such as at least about 5 to about 10 fold or even more.

The expression level of CD62L or LFA-1 determined in or from a sample of a subject can be compared to a single control sample or a plurality of control samples, such as a sample from a control subject, in any suitable manner. As an illustrative example, the expression level of CD62L or LFA-1 in a control sample can be characterized by an average (mean) value coupled with a standard deviation value, for example at a given time point. In some embodiments the expression level of CD62L or LFA-1 in a subject may be considered different when it is one standard deviation or more higher or lower than the average value of the corresponding expression level determined in one or more control samples. In some embodiments the determined expression level of CD62L or LFA-1 is regarded as different where the obtained value is about 1.5 standard deviations higher or lower, including about two, about three, about four or more standard deviations higher or lower than the average value determined in a control sample. In some embodiments the determined expression level of CD62L or LFA-1 is regarded as different where the obtained value is about 1.2 times or more higher or lower, including about 1.5 times, about two fold, about 2.5-fold, about three fold, about 3.5 fold, about 4-fold, about 5-fold or more higher or lower than the expression level determined in a control sample. In some embodiments the determined expression level of CD62L or LFA-1 is regarded as different where the obtained value is about 0.8-fold or less than the expression level determined in a control sample. The determined expression level of CD62L or LFA-1 may for example be regarded as different if a value is about 70%, such as about 60% or about 50% lower than the expression level determined in a control sample. In some embodiments an expression level of CD62L or LFA-1 is regarded as different if the obtained value is about 40%, including about 30% lower than the expression level determined in a control sample. An expression level of CD62L or LFA-1 is in some embodiments regarded as different if the obtained value is about 25%, such as about 20% or lower than the expression level determined in a control sample.

In some embodiments a reduced amount of CD62L or LFA-1 relative to a threshold value, indicates an elevated risk of occurrence of PML in a subject. An amount of CD62L or LFA-1 that is not below a threshold value or that is above a threshold value indicates that there is no elevated risk of occurrence of PML in the subject. A level of CD62L or LFA-1 below a threshold value may indicate a condition where the subject is in need of therapy or in need of a change of a therapy to which the subject is being exposed. If a level of CD62L or LFA-1 is detected that is above a predetermined threshold value, this may indicate that no PML has occurred, as well as that the risk of occurrence of PML is low.

In some embodiments a plurality of measurements is carried out on a plurality of samples from the same patient. In each of the samples the level of expression of CD62L or LFA-1 is determined. Typically the level of expression determined in each of the samples is compared to a threshold value as detailed above. In some embodiments the plurality of samples from the same individual is taken over a period of time at certain time intervals, including at predetermined time intervals. Such an embodiment may be taken as a method of monitoring the expression of CD62L and optionally LFA-1. Matching samples may in some embodiments be used to determine a threshold value for each corresponding time point. The average value may be determined and the standard deviation calculated for each given time point. A value determined in the sample from the subject falling outside of the mean plus 1 standard deviation may be indicative of susceptibility to PML.

In one embodiment the level of CD62L or LFA-1 is measured at certain, e.g. predetermined, time intervals. Samples from the subject may be provided that have been obtained at the corresponding time points. As an illustrative example, samples may be taken from the same subject after a time interval of about 3 months, including about every month. In some embodiments samples may be taken from the same subject at a time interval of about 6 months. In some embodiments a sample may be taken from the same subject after a time interval of about a year, i.e. about 12 months. In some embodiments a sample may be taken from the same subject after about 18 months. A value obtained from a respective sample may in some embodiments be compared to a sample taken from the same subject at a previous point of time, for example the previous measurement and/or the first measurement taken. In this way a change in the level of CD62L or LFA-1 may be detected. Matching samples may in some embodiments be used to determine a threshold value for each corresponding time point. The average value may be determined and the standard deviation calculated for each given time point. As an illustrative example, a value determined in the sample from the subject falling outside of the mean plus 1 standard deviation may for instance be indicative of the occurrence or of the risk of occurrence of PML.

In some embodiments a measurement is repeated if during monitoring, i.e. measuring the amount of CD62L or LFA-1 at certain time intervals an increase or decrease is detected, in particular if an increase or decrease beyond a threshold value is detected. In some embodiments time intervals after which the level of CD62L or LFA-1 are being determined may be shortened if during monitoring of the amount of CD62L or LFA-1 an increase or decrease has been detected. As an illustrative example, a decrease or increase in levels of one or more of CD62L or LFA-1 may have been found at a certain point of time during measurements carried out at intervals of 12 months or during measurements carried out at intervals of 18 months. After such a decrease or increase in levels has been found, monitoring of the level of CD62L or LFA-1 may be continued at time intervals of about a month. As indicated above, monitoring the amount of CD62L or LFA-1 may be included in the context of monitoring a therapy, for example in order to assess the efficacy thereof or to evaluate a subject's response to a certain treatment.

The terms “treatment” and “treating” as used herein, refer to having a therapeutic effect and at least partially alleviating or abrogating an abnormal condition in the organism of a subject. Generally a treatment reduces, stabilizes, or inhibits progression of a symptom that is associated with the presence and/or progression of a disease or pathological condition. The term “administering” relates to a method of incorporating a compound into cells or tissues of a subject. The term “therapeutic effect” refers to the inhibition or activation of factors causing or contributing to the abnormal condition. A therapeutic effect relieves to some extent one or more of the symptoms of an abnormal condition or disease. The term “abnormal condition” refers to a function in the cells or tissues of an organism that deviates from their normal functions in that organism. An abnormal condition can inter alia relate to cell proliferation, cell differentiation, or cell survival.

In embodiments where the subject is to be treated, for example with a VLA-blocking agent, monitoring expression levels may in some embodiments start prior to the treatment. In some embodiments monitoring may start at the same time or at an early stage of the treatment, e.g. administration of VLA-4 blocking agents.

As indicated above, in some embodiments a method according to the present invention includes measuring CD62L and optionally LFA-1 expression on T cell in a sample or obtained from a sample, and comparing the result obtained therefrom to a reference value. In the context of a therapy in some embodiments detecting the level of CD62L expressing T cells as well as monitoring the same includes determining whether one or more of the following indications is present:

(1) In the context of therapy with a VLA-4 blocking agent, a lack of CD62L expression may be observed after administration of a VLA-4 blocking agent. The lack of CD62L expression may be observed at a point of time, such as within about the first week. In some embodiments lack of CD62L expression may be detected within about the second week or within about the third week. A lack of CD62L expression may in some embodiments be detected within about the 1^{st} month, within about 2^{nd} month, within about 3^{rd} month, within about the 4ₜₕ, 5^{th}, 6^{th}, 7^{th}, 8^{th}, 9^{th}, 10^{th}, 11^{th}, 12^{th}, 13^{th}, 14^{th}, 15^{th} or within about the 16^{th} month. In some embodiments a lack of CD62L expression may be detected in the 17^{th} month. In some embodiments lack of CD62L expression may be detected in the 18^{th} month. A lack of CD62L expression may in some embodiments be detected within the 19ₜₕ, the 20^{th}, 21^{st}, 22^{nd}, 23^{rd}, 24^{th}, 25^{th} month or longer. For example, FIG. 8 shows that only very low levels of CD62L in a sample from one subject who later developed PML could be detected after 15 months of treatment.

(2) A differential expression level of CD62L on the T cell surface compared to a reference level obtained from “control subjects”, as indicated above. A differential expression in some embodiments refers to a “decreased” expression compared to a reference. In the context of therapy with a VLA-4 blocking agent control subjects may be defined as those who underwent VLA-4 blocking agent treatment for about one year or more, such as about 1.5 years or more, about 2 years or more, or about 3 years or longer but who have not been diagnosed with PML. The samples to be compared are in some embodiments obtained from the same or substantially the same time point after the initiation of the treatment. For example, a 1-month sample is in some embodiments compared to another 1-month sample. In the context of HIV infection a control subject may be an individual of a comparable stage of AIDS, who is known not to have PML. A “differential” expression is observed by comparing a measured expression level to a corresponding level of one or more control subjects. A “differential” expression, as used throughout the present application, is observed when the expression is lower or higher than that observed from one or more control subjects such that one of skill in the art would consider it to be of statistical significance. In such case, the differential expression is a “decreased” expression compared to a reference.

The expression of CD62L determined from a sample of a subject can be compared to control patients in any suitable manner. For example, the expression of CD62L in the control patient can be characterized by an average (mean) value coupled with a standard deviation value at a given time point. The expression of CD62L in a subject may be considered different when it is more than one standard deviation different from the average value.

As used herein, the term “level” is intended to mean the amount, accumulation or rate of synthesis of the biomarker molecule. A level can be represented, for example, by the amount or synthesis rate of messenger RNA (mRNA) encoded by a gene, the amount or synthesis rate of polypeptide corresponding to a given amino acid sequence encoded by a gene, or the amount or synthesis rate of a biochemical form of a molecule accumulated in a cell, including, for example, the amount of particular post-synthetic modifications of the nucleic acid or polypeptide. The term can be used to refer to an absolute amount of the biomarker protein or mRNA in a sample or to a relative amount of the molecule, including amounts determined under steady-state or non-steady-state conditions. Thus, a CD62L expression level may be represented by the amount of CD62L, accumulation or rate of synthesis of CD62L or the precursor form or a post-translationally modified form of CD62L.

(3) A low number of T cells expressing CD62L. This can be represented by, for example, ratio of such cells to total PBMC, number of cells per sample (e.g. mm³ blood), ratio of such cells to all T cells, or otherwise, as suitable for such representation. When the number is represented by percentage of T cells expression CD62L, a “low” percentage is defined as less than about 10%. In some embodiments a low percentage is defined as less than about 9%, such as less than about 8%, such as less than about 7%, 6%, 5%, 4%, or 3%. A low percentage of T cells expressing CD62L is in some embodiments defined as less than about 2%. %. In some embodiments a low percentage is defined as 1% or less, including about 0.5%, or less. If other methods are employed, a skilled person is able to convert the values here given according to the method used and common knowledge. If the value observed is persistently low over an extended period of time, the subject is more susceptible to PML. “Extended period of time” refers to a period of a plurality of months, such as about 5 months or more. In some embodiments an extended period of time is a period of 6 months or more, such as about 7, 8, 9, 10, or 11 months. In some embodiments an extended period of time is a period of 12 months or longer. As an illustrative example, FIG. 8 shows that the CD62L levels on T cells from subjects who later developed PML were persistently low.

(4) A lack of “recovery” of the percentage of T cells which expresses CD62L. The term “recovery” is determined by comparing the obtained amount or level to a threshold value, which may be based on a reference level. As used herein, “recovery” is defined as a return of the percentage of T cells which express CD62L back to the range of the reference level or higher.

The reference level for this purpose can be determined by various methods. In some embodiments, the reference is obtained from the same subject at the first month of the treatment of VLA-4 blocking agent. In some embodiments, the reference level may be from an earlier point in time, such as about 3 months ago. In some embodiments the earlier point in time may be 4 months ago, such as about 5 months ago. The earlier point in time may in some embodiments be about 6 months ago. In some embodiments the earlier point in time may be about 7 or about 8 months ago, or historical reference level from past course of treatment. In some embodiments, the reference level is obtained from one or more control subjects, such as about 30 or more control subjects who underwent treatment of VLA-4 blocking agent for more than 1 year. The reference level is in some embodiments obtained from about 40 or more control subjects, including about 50 or more, about 60 or more or about 70 or more control subjects who underwent treatment of VLA-4 blocking agent for more than 1 year, such as more than about 1.5 years, more than about 2 years, about 2.5 years, about 3 years, or more. In some embodiments, the reference level is measured within about the first month after the first administration of the VLA-4 blocking agent.

FIG. 8 shows that the CD62L levels of control subjects recovered (exceeding the reference level taken at the first month) after 15 months of VLA-4 blocking agent treatment.

In some embodiments, a sample from the subject to be tested is taken about one month after the treatment. PMBC is isolated from the sample and subjected to a suitable detection technique such as FACS analysis. The percentage of T cells, including CD4⁺ T cells and/or CD8⁺ T cells, which are CD62L positive is measured and compared to a reference level derived from one or more control subjects. If the measured value is lower or higher than the threshold value, it is indicative of an increased susceptibility to PML.

For instance, the following indicate reference levels for CD62L that can be used to set a threshold value:

TABLE 1
Exemplary reference values for CD62L for individuals receiving
Natalizumab

| | % of CD4⁺ T cells | |
|---|---|---|
| | positive for | reference level (mean % of |
| | CD62L (mean | CD4⁺CD62L⁺ T cells minus |
| Month | (standard deviation)) | 1 standard deviation) |
| 0 (before treatment) | 53.7 (10.2) | 43.5 |
| 1 | 28.2 (7.2) | 21.0 |
| 3 | 36.9 (17.8) | 19.1 |
| 6 | 20.0 (14.9) | 5.1 |
| 12 | 16.7 (16.2) | 0.5 |
| 15-20 | 40.0 (13.9) | 26.1 |
| 21-25 | 41.8 (10.3) | 31.5 |
| 26-30 | 44.9 (12.5) | 32.4 |
| 31-35 | 34.0 (6.9) | 27.1 |
| 36-40 | 38.0 (18.1) | 19.9 |
| 41-45 | 33.5 (6.4) | 27.1 |
| 46-50 | 32.3 (2.3) | 30.0 |
| 51-55 | 39.5 (20.7) | 18.8 |

By way of example, the reference level for a subject having received 1 month treatment of Natalizumab may be 21%. An expression level lower than 21% may be considered “different” and indicate a risk for PML.

When one of the above indications is observed, for example, when there is a lack of CD62L expression, or when low expression of CD62L persists for an extended period of time, the physician should consider, combined with other information available, stopping or temporarily withholding the treatment, adjusting the dosage, performing plasma exchange, or the like until the expression level increases or recovers. It may be possible to resume the treatment after the expression level of the biomarkers in the present invention has recovered or increased.

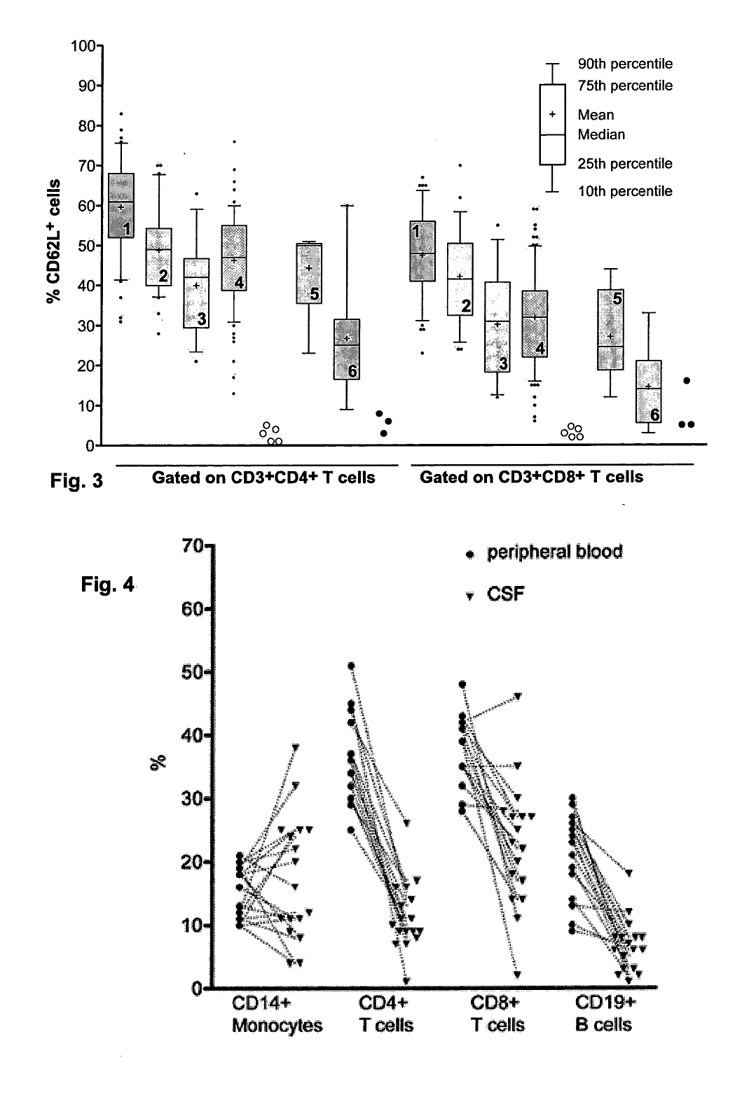

As can be taken from FIG. 3 and FIG. 8, levels of CD62L on T cells tend, with the exception of about the initial 12 months of treatment at all, to remain within a relatively stable range during treatment of relapsing remitting multiple sclerosis with a VLA-4 blocking agent. A drop of CD62L on T cells can typically be observed after onset of PML (cf. also FIG. 8). It is thus in some embodiments helpful to monitor the time course of CD62L levels on T cells of an individual. In this way any unexpected alteration of CD62L levels can be detected. Such alteration is an indication of an elevated risk of PML.

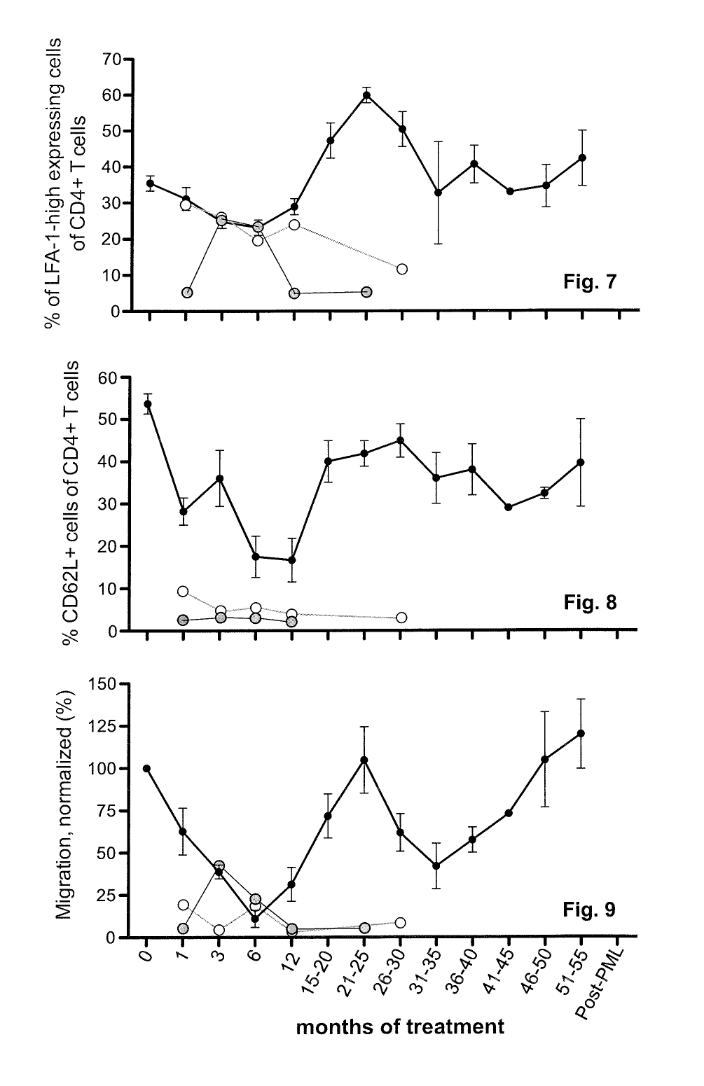

As explained above, in some embodiments of a method or use of the invention the expression level of LFA-1 in the sample is determined. In some embodiments the expression level of LFA-1 is determined at a plurality of time points, for example by determining the expression level of LFA-1 in a plurality of samples, which have been obtained from the same subject at particular time points over a period of time. If the expression level of LFA-1 observed is persistently different from a threshold value over an extended period of time, the subject is more susceptible to PML. As an example in this regard, FIG. 7 shows that the LFA-1 levels of two patients who later developed PML were persistently lower than that from control patients after month 6 and 12.

For instance, a reference level of LFA-1 as indicated in the following can be used to set a threshold value:

TABLE 2
Exemplary reference values for LFA-1 for individuals receiving
Natalizumab

| | % of CD4⁺ T cells positive | reference level (mean % of |
|---|---|---|
| | for LFA-1 (mean (standard | CD4+LFA-1⁺ T cells minus 1 |
| Month | deviation)) | standard deviation) |
| 0 (before | 35.5 (13.6) | 21.9 |
| treatment) | | |
| 1 | 31.2 (12.7) | 18.5 |
| 3 | 25.4 (7.6) | 17.8 |
| 6 | 23.8 (9.8) | 14.0 |
| 12 | 28.9 (10.2) | 18.7 |
| 15-20 | 47.3 (16.3) | 31.0 |
| 21-25 | 59.9 (7.5) | 52.4 |
| 26-30 | 50.4 (16.8) | 33.6 |
| 31-35 | 26.0 (19.8) | 6.2 |
| 36-40 | 40.6 (18.9) | 21.7 |
| 41-45 | 37.0 (5.7) | 31.3 |
| 46-50 | 34.5 (11.8) | 22.7 |
| 51-55 | 42.2 (17.2) | 25.0 |

For the purpose of the present invention, the detection of LFA-1 expression can also include detecting the protein or mRNA of CD11a and Runx3. In this case, the determination of susceptibility may be carried out using generally the same approach for LFA-1 protein.

As should be apparent from the above, if for example a level of T cells that have CD62L and/or both CD62L and LFA-1 is detected that is above or below a (e.g. predetermined) threshold value, this may indicate a risk that the subject will have PML, generally at a later point of time. In embodiments where the sample is from an HIV positive subject, if a level of T cells that have both CD62L and LFA-1 is detected that is above or below a predetermined threshold value, this may indicate the need to change therapy. A level of T cells that have both CD62L and LFA-1 above or below a predetermined threshold value may also indicate a condition where the subject is suffering from PML. In case it is suspected that a subject is suffering from PML the practitioner will usually carry out MRI imaging. It may for example be analysed whether lesions in subcortical white matter exist. The presenting PML symptoms most commonly include changes in cognition, behaviour, and personality, but in some cases seizures may be the first clinical event. Such symptoms may occur either alone or associated with motor, language, or visual symptoms.

If the expression level of CD62L on T cells from a subject is detected that is above or below a threshold value, this may also indicate a risk that the subject will have PML. If the expression level of CD62L observed is for instance persistently different from a threshold value over an extended period of time, the subject is diagnosed to be at an elevated risk to develop PML.

In some embodiments of a method according to the invention prior to a planned treatment the level of CD62L on T cells from a subject is determined as detailed above. If an increased or a decreased level of CD62L present on T cells, relative to a threshold value, is determined, an increased risk of PML occurrence may be diagnosed. In embodiments where the subject is HIV positive the planned therapy may be adjusted in order to achieve a particularly fast and effective immune restoration and/or in order to assist the subject's organism to provide JCV specific T cell responses. In some embodiments it may be considered to include a HT₂ₐ antagonist into a planned therapy. As indicated above, in some embodiments the level of CD62L on T cells from a subject may be monitored over time. For this purpose frozen samples that were obtained from the subject at different time points may for instance be analysed within the same measurement. The level of CD62L on T cells may for instance be measured at time intervals of one or more months such as about every 6 months, about every 8 months, about every 10 months, about every 12 months or about every 14 months during a treatment, for instance with a VLA-4 blocking agent, or as long as the subject is diagnosed to suffer from a disease such as HIV or multiple sclerosis. A change of the level of CD62L on T cells may indicate that the subject is at a risk of developing PML. Depending on further diagnosis results, a change of the level of CD62L on T cells may also indicate that the subject is developing PML. In some embodiments where the subject is undergoing treatment with a VLA-4 blocking agent the level of CD62L on T cells may be determined before a treatment with a VLA-4 blocking agent is begun. Thereafter a further analysis of the level of CD62L on T cells may for instance be carried out about 1.5 years after the start of treatment. Subsequently the level of CD62L on T cells from the subject may be analysed about every 6 months.

Methods according to the present invention can be used to predict whether a subject is likely to develop PML. This is of particular importance since no PML therapy is currently available and overall mortality is above 50%, as explained above. In addition, once PML is diagnosed in a subject undergoing treatment with a VLA-4 blocking agent, plasma exchange or immunoadsorption is required in order to more rapidly remove the VLA-4 blocking agent from plasma and to speed up the reconstitution of immune surveillance. In this regard immunoadsorption is only established as a medical procedure in Europe and Japan, but not in North America. The reconstitution of immune function following removal of e.g. a monoclonal immunoglobulin with plasma exchange procedures, or immune reconstitution with HAART, is often accompanied by an exaggerated pathological inflammatory response termed immune reconstitution inflammatory syndrome (IRIS), also known as “restoration disease (IRD)”, “immune reconstitution syndrome (IRS)”, “immune recovery disease”, and “immune rebound illness”. As the immune system recovers, influx of cytotoxic and bystander lymphocytes eliminates infected oligodendrocytes and augments bystander inflammation. The immune system has been postulated to respond to a previously acquired opportunistic infection with an overwhelming inflammatory response that paradoxically renders symptoms of infection worse. Since IRIS has been found to occur in the absence of any apparent active infection, it has also been postulated to arise merely due to restoration of the previously suppressed inflammatory immune response due to reactivation of memory cells that had been previously activated by antigen exposure. IRIS typically leads to clinical deterioration, causing high disability and mortality. IRIS was first described in patients with HIV, however it is more common in MS patients treated with Natalizumab.

In HIV infected subjects IRIS typically develops within weeks or months (Post, M. J. D., et al., Am. J. Neuroradiol. (2013) 10.3174/ ajnr.A3183). IRIS significantly negatively impacts the HIV infected population on HAART by increasing the number of procedures, number of hospitalizations, and the overall morbidity in this patient cohort (ibid.). Among JCV positive HIV infected patients that have been treated with HAART, it has been reported that 18% may develop IRIS (ibid.). In HIV negative patients on immunomodulatory therapy such as natalizumab, PML-IRIS is reportedly more severe than in HIV infected patients due to the restored immune surveillance in the latter (ibid.).

IRIS is a robust inflammatory response, which may occur as a mild disease, but also as a life-threatening deterioration. A method according to the invention allows early prediction of the risk of PML occurrence and therefore provides time to adjust treatment before onset of PML. Thus occurrence of IRIS may be avoided and thereby a potential additional health/life risk be circumvented.

A method as described above may also be a method of assessing the occurrence of PML. In such an embodiment the subject from whom/which the sample originates is generally suspected to suffer from PML. An increased amount of CD62L and/or LFA-1, relative to the threshold value, indicates the presence of PML. A method as described above may further in some embodiments be a method of assessing the chances of survival from PML in a subject. The subject is generally known to have PML. An increased or decreased level of CD62L expressing T cells, relative to the threshold value, may indicate low chances of survival of PML.

The present invention further provides a method of treating a subject having a demyelinating disease or an autoimmune disease. The method includes administering a VLA-4 blocking agent to the subject. Typically the method also includes monitoring the expression of at least one biomarker on T cells, with the monitoring being carried out on a sample from the subject. A respective biomarker may be CD62L and/or LFA-1. The method may also include determining the migratory capacity of CD45⁻CD49d⁺ immune cells. The VLA-4 blocking agent can be used to treat a number of diseases and disorders, including multiple sclerosis, Crohn's disease, rheumatoid arthritis, meningitis, neuromyelitis optica, neurosarcoidosis, CNS vasculitis, chronic inflammatory demyelinating polyradiculo-neuropathy (CIDP), encephalitis, transverse myelitis, tissue or organ graft rejection or graft-versus-host disease, chronic renal disease, CNS injury, e.g., stroke or spinal cord injury; chronic renal disease; allergy, e.g., allergic asthma, type 1 diabetes, an inflammatory bowel disorder, e.g., ulcerative colitis, myasthenia gravis, fibromyalgia, arthritic disorders, e.g., rheumatoid arthritis or psoriatic arthritis, an inflammatory/immune skin disorder, e.g., psoriasis, vitiligo, dermatitis or lichen planus, systemic lupus erythematosus, Sjogren's Syndrome, a hematological cancer, e.g., multiple myeloma, leukemia or lymphoma, a solid cancer, e.g., a sarcoma or a carcinoma, e.g., of the lung, breast, prostate, brain, as well as a fibrotic disorder, e.g., pulmonary fibrosis, myelofibrosis, liver cirrhosis, mesangial proliferative glomerulonephritis, crescentic glomerulonephritis, diabetic nephropathy, and renal interstitial fibrosis. Any disease or pathological condition which has been treated or is known to be treatable by the blocking agent may be treated in the context of the present invention.

Typically the treatment of a subject having a demyelinating disease or an autoimmune disease includes administering a therapeutically effective amount of a VLA-4 blocking agent. As used herein, the term therapeutically effective amount of VLA-4 blocking agent is an amount sufficient to provide a therapeutic benefit in the treatment or management of the relevant pathological condition, or to delay or minimize one or more symptoms associated with the presence of the condition. The blocking agent may, for example, be administered intravenously. For Natalizumab, the dose may be 1 to 6 mg per kilogram of body weight. In one embodiment, a standard does of 300 mg Natalizumab diluted with 100 ml 0.9% sodium chloride is injected intravenously once every four weeks. The dose may be repeated at intervals from two to eight weeks. For example, a treatment regimen may include 3 mg Natalizumab per kg body weight repeated at about a four week interval. A skilled person in the art is capable of determining the therapeutic effective amount and the methods are also described in the references incorporated herein.

A subject may be first subjected to prior screening to determine whether the planned treatment would be suitable. For example, such a screening may be based on the patient history, previous use of immunosuppressant, Expanded Disability Status Scale (EDSS) in case of multiple sclerosis patients, anti-JCV antibody status (JCV antibody seropositivity), MRI imaging studies, pre-infusion checklist for continuously worsening neurological symptoms, and other criteria commonly used.

Typically a subject undergoing VLA-4 blocking agent treatment is tested to determine the expression level of a biomarker as disclosed herein, e.g. the expression level of CD62L on T cells in or from a sample of the subject. As a further example, the migratory capacity of CD45⁻CD49d⁺ immune cells may be determined. A method according to the invention may also include any other molecule or effect that can be used to indirectly indicate the level of such biomarker. One or more samples from the subject are collected and analyzed. In some embodiments the one or more samples are sent to a central testing facility to ensure that the analysis of phenotype and function can be carried out under standardized conditions. Samples may be taken and tested prior to the treatment and then regularly after the treatment begins, such as monthly, bimonthly, quarterly, every six months, and yearly. The routine assessment for PML provides timely information regarding the safety issues related to the treatment. In one embodiment, the samples are taken at month 1, every 3 months until the first year, and then every 6 months thereafter.

In some embodiments treating the subject undergoing treatment with a VLA-4 blocking agent includes determining, including monitoring, the expression level of CD62L and/or LFA-1 on T cells in or from a sample of the subject. If any indication is found that suggests an increased susceptibility to PML or other complications, or renders such complication more likely than in other subjects, further tests may be carried out. Subjects showing compromised immune surveillance should be clinically monitored very closely. The physician may test the patient for further biomarkers such as those provided in the present invention or known biomarkers, such as anti-JCV antibody status or other clinical or MRI criteria. Based on the information, the practitioner will assess whether to continue, restart or stop the treatment of VLA-4 blocking agents. The information provides significant information to the physician regarding the risk associated with the treatment, so that informed benefit-risk decisions can be made accordingly. As an illustrative example, the monitoring may at the beginning include only determining the level of CD62L expression. When the result indicates an alteration, including a low expression level, compared to the reference value as described above, the LFA-1 expression level and/or the migration capacity of T cells may be tested.

In some embodiments, a reference value or level can also be gathered from patients who suffered from PML as a result of VLA-4 blocking agent treatment. Expression levels of the biomarkers from the PML patients are recorded over a period of time, such as over 2-3 years. Average expression levels, standard deviation, and relative standard deviation at given times are calculated for the patients to determine a range of expression levels associated with PML patients. When test result from a patient to be evaluated is collected, it will be compared to the reference value. Statistical differences between the test result and the reference will be determined to identify significant variances in between.

Accordingly, determining the expression level of CD62L and/or LFA-1 can be used to stratify a subject undergoing or about to undergo treatment with a VLA-4 blocking agent for suspension of the treatment. The terms “stratifying” and “stratification” as used herein indicate in one aspect that individuals are assigned to groups with similar characteristics such as at a similar risk level of developing PML. As an illustrative example, individuals may be stratified into risk categories. The terms “stratifying” and “stratification” as used herein indicate in another aspect that an individual is assigned to a certain group according to characteristics matching the respective group such as a corresponding risk level of developing PML. The groups may be, for example, for testing, prescribing, suspending or abandoning any one or more of a drug, surgery, diet, exercise, or intervention. Accordingly, in some embodiments of a method or use according to the invention a subject may be stratified into a subgroup of a clinical trial of a therapy. As explained in the forgoing, in the context of the present invention CD62L and/or LFA-1 may be used for PML risk stratification.

The terms “stratifying” and “stratification” according to the invention generally include identifying subjects that require an alteration of their current or future therapy. The term includes assessing, e.g. determining, which therapy a subject likely to suffer from PML is in need of. Hence, in the context of the present invention stratification may be based on the probability (or risk) of developing PML. A method or use according to the invention may also serve in stratifying the probability of the risk of PML or the risk of any PML related condition for a subject. A method of stratifying a subject for PML therapy according to the invention includes detecting the amount of determining the expression level of CD62L and/or LFA-1 as described above, and/or assessing the migratory capacity of CD45⁺CD49d⁺ immune cells of the subject. As explained above, in some embodiments on a general basis a CD62L and/or a LFA-1 capture probe can be advantageously used to screen risk patients which have higher susceptibility to PML.

In this regard the use of biomarkers for stratification of patients is a well-established procedure in the art. This procedure includes or consists of linking one or more patient subpopulations, characterized by a certain feature, in the context of the present invention the expression level of a particular protein or migratory capacity of cells, to a particular treatment. The general aim of stratification is to match patients with therapies that are more likely to be effective and safe. In a more general context stratifying patients may include evaluation of patient history and physical assessment, combined with laboratory tests on the basis of a method of the present invention, and clinical observation. It is understood that stratifying patients is only feasible if multiple treatment options with heterogeneous responses for the disease exist. In the context of the present invention HIV therapy may be adjusted or treatment with a VLA-4 blocking agent be suspended for a certain period of time, such as one or more months. A general overview of patient stratification and stratified medicine has been given by Trusheim, M. R., et al., Nature Reviews Drug Discovery (2007) 6, 4, 287-293.

The present invention also provides a method of treating a subject. The method includes administering a VLA-4 blocking agent to the subject. The method further includes determining the expression of one or more biomarkers on one or more T cells from the subject, such as CD4⁺ T cells or CD8⁺ T cells. The biomarker is generally CD62L or LFA-1. In some embodiments the expression of one or more biomarkers on one or more T cells from the subject may be monitored. In some embodiments the method further includes determining, including monitoring, the migration of CD45⁻CD49d⁺ immune cells.

The term “administering”, as used herein, refers to any mode of transferring, delivering, introducing, or transporting matter such as a compound, e.g. a pharmaceutical compound, or other agent such as an antigen, to a subject. Modes of administration include oral administration, topical contact, intravenous, intraperitoneal, intramuscular, intranasal, or subcutaneous administration (cf. below).

A VLA-4 blocking agent or an antiviral agent can be administered to a human patient per se, or in pharmaceutical compositions where they are mixed with other active ingredients, as in combination therapy, or suitable carriers or excipient(s). Exemplary routes include, but are not limited to, oral, transdermal, and parenteral delivery.

Suitable routes of administration may, for example, include depot, oral, rectal, transmucosal, or intestinal administration; parenteral delivery, including intramuscular, subcutaneous, intravenous, intramedullary injections, as well as intrathecal, direct intraventricular, intraperitoneal, intranasal, or intraocular injections.

Alternately, one may administer the compound in a local rather than systemic manner, for example, via injection of the compound directly into a solid tumor, often in a depot or sustained release formulation. Furthermore, one may administer the drug in a targeted drug delivery system, for example, in a liposome coated with a blood-cell specific antibody. The liposomes will be targeted to and taken up selectively by the respective cells.

Pharmaceutical compositions that include the compounds of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers including excipients and auxiliaries that facilitate processing of the active compounds into preparations that can be used pharmaceutically. Proper formulation is dependent upon the route of administration chosen.

For injection, the agents of the invention may be formulated in aqueous solutions, for instance in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated.

Pharmaceutical preparations for oral use can be obtained by adding a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP).

If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

Alternatively, the active ingredient may be in powder form for constitution with a suitable vehicle, e.g., sterile pyrogen-free water, before use. The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as emulsion in an acceptable oil) or ion exchange resins, or as sparingly soluble derivatives, for example, as a sparingly soluble salt.

A pharmaceutical carrier for the hydrophobic compounds of the invention is a co-solvent system including benzyl alcohol, a non-polar surfactant, a water-miscible organic polymer, and an aqueous phase. The co-solvent system may be the VPD co-solvent system. VPD is a solution of 3% w/v benzyl alcohol, 8% w/v of the non-polar surfactant polysorbate 80, and 65% w/v polyethylene glycol 300, made up to volume in absolute ethanol. The VPD co-solvent system (VPD:D5W) consists of VPD diluted 1:1 with a 5% dextrose in water solution.

This co-solvent system dissolves hydrophobic compounds well, and itself produces low toxicity upon systemic administration. Naturally, the proportions of a co-solvent system may be varied considerably without destroying its solubility and toxicity characteristics.

Furthermore, the identity of the co-solvent components may be varied: for example, other low-toxicity non-polar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various types of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

A pharmaceutical composition also may include suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Pharmaceutical compositions suitable for use in the present invention include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

For any compound used in the methods of the invention, the therapeutically effective dose can be estimated initially from cell culture assays. For example, a dose can be formulated in animal models to achieve a circulating concentration range that includes the IC₅₀ as determined in cell culture (i.e., the concentration of the test compound which achieves a half-maximal inhibition of the desired activity). Such information can be used to more accurately determine useful doses in humans.

Toxicity and therapeutic efficacy of the compounds described herein can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio between LD₅₀ and ED₅₀. It may be desired to use compounds that exhibit high therapeutic indices. The data obtained from these cell culture assays and animal studies can be used in formulating a range of dosage for use in humans. The dosage of such compounds lies typically within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition.

Dosage amount and interval may be adjusted individually to provide plasma levels of the active moiety which are sufficient to maintain the kinase modulating effects, or minimal effective concentration (MEC). The MEC will vary for each compound but can be estimated from in vitro data; e.g., the concentration necessary to achieve 50-90% inhibition of the kinase. Dosages necessary to achieve the MEC will depend on individual characteristics and route of administration. However, HPLC assays or bioassays can be used to determine plasma concentrations.

Dosage intervals can also be determined using MEC value. Compounds should be administered using a regimen that maintains plasma levels above the MEC for 10-90% of the time, for example from about 30 to about 90%, such as from about 50 to about 90%.

In cases of local administration or selective uptake, the effective local concentration of the drug may not be related to plasma concentration. The amount of composition administered will, of course, be dependent on the subject being treated, on the subject's weight, the severity of the affliction, the manner of administration and the judgment of the prescribing physician.

A suitable composition may, if desired, be presented in a pack or dispenser device which may contain one or more unit dosage forms containing the active ingredient. The pack may for instance include metal or plastic foil, such as a blister pack. The pack or dispenser device may be accompanied by instructions for administration. The pack or dispenser may also be accompanied with a notice associated with the container in a form prescribed by a governmental agency regulating the manufacture, use, or sale of pharmaceuticals, which notice is reflective of approval by the agency of the form of the compound for human or veterinary administration. Such notice, for example, may be the labelling approved by the U. S. Food and Drug Administration or other government agency for prescription drugs, or the approved product insert.

As already noted above, in some embodiments a method according to the invention includes determining, including monitoring, the migration of immune cells expressing CD45 and CD49d. In some of these embodiments the subject of whom the sample has been obtained is undergoing treatment with a VLA-4 blocking agent. Generally, CD45d is expressed on all leukocytes, and CD49d is expressed on T cells, B cells, monocytes, eosinophils and basophils. In some embodiments the immune cell to be tested is T cell. In one embodiment the T cell is a CD4⁺ T cell. In one embodiment the T cell is a CD8⁺ T cell.

Migratory capacity of immune cells is a prerequisite for immune reactions. A respective method of the invention which can be used to evaluate a subject's immune competence and susceptibility to PML. Any technique that is suitable for determining the migratory capacity of an immune cell can be used. This can be done using any known techniques in the art. In some embodiments a chemotaxis assays is employed. Such assays are based on the functional migration of cells induced by a compound, and can be used to assess the binding and/or chemoattractant effect of e.g. ligands, inhibitors, or promoters. The use of an in vitro assay is illustrated in the Examples and also disclosed in U.S. Pat. No. 5,514,555. In some embodiment chemotaxis assay determines the migration across endothelium into a collagen gel (described in Kavanaugh et al, J. Immunol (1991) 146, 4149-4156). Such assay may involve the use of a transwell-based set-up. In some embodiments a chemoattractant is dissolved in the medium on one side of a migratory barrier such as a polymeric gel. On the other side of the migratory barrier the cells of the sample from the patient are positioned. The migratory barrier is porous to a certain extent so that the cells of interest such as T cells are able to migrate through the same. The pores of the porous migratory barrier further allow the passage of chemoattractant molecules, so that a diffusion gradient forms, which can be detected by the cells of interest. As a result the cells are attracted to migrate across the migratory barrier. Typically the cells are allowed to migrate in the experimental setup for a certain, e.g. predetermined, period of time, whereafter the number of migrated cells and/or the migration distance is being determined. For this purpose the migratory barrier may be analyzed under a microscope. The cells may also be stained before starting the chemotaxis assay and their position may be determined according to the signals obtained from the stain.

In one embodiment, the migratory capacity is compared to that obtained from the same patient prior to the treatment of VLA-4 blocking agent. The value obtained can be set to 100%. After treatment is initiated, a drop in immune cell migration can be observed and compared. The migration at a given time point can be characterized by an average (mean) value coupled with a standard deviation value. Cell migration in a subject may be considered different when it is more than one standard deviation different from the average value (supra). The reference value may be defined as the mean minus 1 standard deviation. When the difference falls below the reference value, it may be indicative of an increased risk for PML occurrence in the subject. The above said with regard to a threshold value in this regard applies mutatis mutandis.

As an illustrative example the following table provides an exemplary reference level for immune cell migration that may be used. In this instance, migration of CD3⁺ T cells over endothelium has been monitored over a period of time.

TABLE 3
Exemplary reference values for migration of CD3⁺ T cells

| | % of migrated CD3⁺ T cells in | reference level (mean % |
|---|---|---|
| | relation to untreated | of migrated CD3⁺ T cells |
| | patients (set to 100%) (mean | minus 1 standard |
| Month | (standard deviation)) | deviation) |
| 0 (before | 100.0 (none) | |
| treatment) | | |
| 1 | 62.7 (27.5) | 35.2 |
| 3 | 38.8 (7.0) | 31.8 |
| 6 | 11.1 (11.3) | 0 |
| 12 | 31.3 (22.3) | 9.0 |
| 15-20 | 71.7 (38.9) | 32.8 |
| 21-25 | 104.7 (61.8) | 42.9 |
| 26-30 | 61.8 (36.9) | 24.9 |
| 31-35 | 35.7 (22.7) | 13.0 |
| 36-40 | 57.5 (25.7) | 31.8 |
| 41-45 | 57.0 (22.6) | 34.4 |
| 46-50 | 104.6 (48.8) | 55.8 |
| 51-55 | 119.8 (45.6) | 74.2 |

Immune cells have a basic capacity to migrate over cellular barriers and permeable membranes. The inventors have found that a lack or reduced CD62L expression is associated with a strongly reduced migratory capacity. A migration assay used in a method according to the present invention may involve the use of a permeable membrane. The membrane may be any membrane commonly used in the field, such as polycarbonate (PC), polyester (PET), and collagen-coated polytetrafluoroethylene (PTFE) membrane, which are available commercially (for example Transwell® membrane). A migration assay over a blank permeable membrane, i.e. without cells, may be used for such assessment. In some embodiments a migratory assay involves the use of cells. Cells such as endothelial cell or cell lines are within the scope of the present invention. Exemplary cells are -end cell line, c-end cell line, Human umbilical vein endothelial cell (HUVEC) cell line. In some embodiments primary human choroid plexus-derived epithelial cells are employed. In some embodiments primary human brain microvasculary endothelial cells are used.

In addition, further known biomarkers can be optionally used as secondary markers in the context of the present invention to assist the assessment of the susceptibility to PML of a patient receiving VLA-4 blocking agents. Such markers include the treatment duration, pretreatment with immunosuppressants, as well as the serum-positivity of JCV antibodies. In another aspect, the present invention provides a method of treating a patient comprising administering the patient with VLA-4 blocking agents, and measuring or detecting the level of expression of CD62L from the T cell, and stopping or continuing the administration based on the level of expression. As described earlier, the level of CD62L expression can be used to indicate risk or occurrence of PML. Thus, if the level indicates that there is an elevated risk of PML, the treatment should be stopped; otherwise, the treatment may continue.

VLA-4 blocking agent can be used to treat a number of diseases and disorders, including multiple sclerosis, Crohn's disease, rheumatoid arthritis, meningitis, neuromyelitis optica, neurosarcoidosis, CNS vasculitis, chronic inflammatory demyelinating polyradiculoneuropathy (CIDP), encephalitis, transverse myelitis, tissue or organ graft rejection or graft-versus-host disease, chronic renal disease, CNS injury, e.g., stroke or spinal cord injury; chronic renal disease; allergy, e.g., allergic asthma; type 1 diabetes; inflammatory bowel disorders, e.g., ulcerative colitis; myasthenia gravis; fibromyalgia; arthritic disorders, e.g., rheumatoid arthritis, psoriatic arthritis; inflammatory/immune skin disorders, e.g., psoriasis, vitiligo, dermatitis, lichen planus; systemic lupus erythematosus; Sjogren's Syndrome; hematological cancers, e.g., multiple myeloma, leukemia, lymphoma; solid cancers, e.g., sarcomas or carcinomas, e.g., of the lung, breast, prostate, brain; and fibrotic disorders, e.g., pulmonary fibrosis, myelofibrosis, liver cirrhosis, mesangial proliferative glomerulonephritis, crescentic glomerulonephritis, diabetic nephropathy, and renal interstitial fibrosis.Any disease or pathological condition which has been treated or is known to be treatable by the blocking agent is part of the present invention. Accordingly, the treatment comprises administering a therapeutically effective amount of VLA-4 blocking agents. Patients may be first subjected to prior screening to determine whether the treatment would be suitable. For example, the screening may be based on the patient history, previous use of immunosuppressant, Expanded Disability Status Scale (EDSS) in case of multiple sclerosis patients, anti-JCV antibody status (JCV antibody seropositivity), MRI imaging studies, pre-infusion checklist for continuously worsening neurological symptoms, and other criteria commonly used.

Patient undergoing VLA-4 blocking agent treatment is tested to determine the expression level of the biomarkers as disclosed herein. Samples from the patient are collected and analyzed. Preferably, they are sent to a central testing facility to ensure that the analysis of phenotype and function can be carried out under standardized conditions. In a preferred embodiment, the samples are taken at month 1, every 3 months until the first year, and then every 6 months thereafter. In one embodiment, the treatment should be stopped if there is a lack of CD62L expression on T cells since this indicates an increased risk occurrence of PML.

The determination whether to stop the treatment or not can be based on comparing the level of expression with a reference. As disclosed herein, the reference may be derived from one or more patients known to have suffered from PML or other complications, or one or more patients known to have not suffered from PML or other complications. For example, reference value or level can be gathered from control patients. Expression levels of the biomarkers from the control patients using any suitable method may be recorded over a period of time, such as over 2-3 years. Average expression levels, standard deviation, and relative standard deviation at given times can be calculated for the control patients to determine a range of expression levels associated with the control patients. When test result from a patient to be evaluated is collected, it will be compared to the reference value. Statistical differences between the test result and the reference will be determined to identify significant variances In between.

may be provided in the Based on CD62L expression, the physician is able assess whether to continue, restart or stop the treatment of VLA-4 blocking agents. The information provides significant information to the physician regarding the risk associated with the treatment, so that informed benefit-risk decisions can be made accordingly.

It is to be understood that the methods of the present invention cannot provide a 100% prediction whether or not a patient will develop PML or other complications, since individual factors such as other preexisting condition, general health, drug interaction and the like may have an influence as to whether or not a patient will be susceptible to PML

In addition, if a CD62L expression level is detected that indicates that a subject is at an elevated risk of PML occurrence, diagnosis with regard to PML may be intensified. As further explained below, MRI imaging may be employed to identify any area of demyelination. Further, cerebrospinal fluid may be analyzed for the presence of JCV DNA, or blood or a brain sample may be analyzed with regard to the presence of TNFR1 or TNF-α. If any of these diagnostic measures have previously been carried out on the subject, including carried out on a regular basis, if on the basis of CD62L expression levels, a subject is found to be at an elevated risk of developing PML, one or more such means of diagnosing PML may be carried out on a regular basis, including on a more frequent basis than previously done. As an illustrative example, it may be decided by the physician that every three months MRI imaging is carried out on the subject's brain.

In some embodiments the treatment includes administering a VLA-4 blocking agent to the subject. The method further includes measuring or detecting the level of expression of CD62L on T cells of the subject. Based on the level of expression of CD62L on the subject's T cells the administration of the VLA-4 blocking agent is stopped or continued. A threshold value may be used as a decision threshold (supra). If a CD62L expression level is detected that indicates that there is no elevated risk of PML occurrence, the administration of the VLA-4 blocking agent may be continued. If a CD62L expression level is detected that indicates that there is an elevated risk of PML occurrence, the administration of the VLA-4 blocking agent should be stopped. In some embodiments measures are taken to remove the VLA-4 blocking agent from the subject's plasma if an elevated risk of PML occurrence has been determined. As explained above plasma exchange or immunoadsorption may be carried out in this regard. In some embodiments stopping the administration of a VLA-4 blocking agent means that therapy with a VLA-4 blocking agent is entirely stopped, i.e. no alternative VLA-4 blocking agent is administered instead of the previously administered VLA-4 blocking agent. Entirely ending or adjourning therapy with a VLA-4 blocking agent may assist reconstitution of the subject's immune surveillance. It is further noted in this regard that a subject suffering from MS and under therapy with a VLA-4 blocking agent is often a subject that/who did not respond to a first-line therapy such as interferon-β or glatiramer acetate. Beginning such a therapy as a substitute of VLA-4 blocking agent therapy may therefore only have a low chance of improving the subject's condition.

In another aspect the present invention provides a kit. Such kit includes an assay kit for detecting one or more biomarkers as provided in the present invention. Means for detecting a biomarker are known in the art, and include, for example, the use of a capture probe such as an immunoglobulin or a proteinaceous binding molecule with immunoglobulin-like functions, which optionally is detectably labeled. As explained above the capture probe may be used together with a detection agent that binds to the biomarker and/or the capture probe. A capture probe may be, for example and not by limitation, an antibody, a portion of an antibody such as a Fab or Fab2 fragment, a single chain antibody, a receptor for the biomarker or a portion thereof or a ligand for the biomarker or a portion thereof. Likewise, a detection agent may be, for example and not by limitation, an antibody, a portion of an antibody such as a Fab or Fab2 fragment, a single chain antibody, a receptor for the biomarker or capture agent or a portion thereof or a ligand for the biomarker or capture probe or a portion thereof. The capture probe and/or detection agent may be detectably labeled using a radioactive label, a fluorescent label, a chemical label, an oligonucleotide label, an enzymatic label, or a protein label (e.g. a fluorescent protein such as Green Fluorescent Protein). It is to be understood that the method may also include any molecules which can be used to indirectly indicate the level of the biomarkers. A “capture probe” may be a molecule that binds an mRNA or protein through an interaction that is sufficient to permit the binding to the mRNA or protein.

In one embodiment the kit may include a CD62L specific capture probe, and optionally a LFA-1 capture probe. The kit may further include a CD3 specific capture probe. In some embodiments the kit may further include a CD4 specific capture probe and/or a CD8 specific capture probe. In some embodiments the kit may include a multi-specific capture probe directed to CD3, CD62L and LFA-1, optionally together with a detection agent. In one embodiment the kit includes components for setting up an assay for CD3 and CD62L. In some embodiments the kit includes an immunoglobulin or a proteinaceous binding molecule with immunoglobulin-like functions, or any other capture probe directed to the protein or mRNA of CD11a, and a capture probe directed to the protein or mRNA of CD18. Such a kit may also include a capture probe directed to the protein or mRNA of Runx3.

A respective kit may furthermore include means for immobilising the capture probe to a surface. As explained above, a nucleic acid capture probe included in the kit may have a moiety that allows for, or facilitates, an immobilisation on a surface.

The kit may further include instructions and/or imprint indicating that a patient is to be stratified by a method described herein; and/or instructions regarding how to carry out a method as defined herein. It may also include positive and/or negative controls which allow a comparison to the control. The kit shall enable the assessment of a patient's treatment progress and the susceptibility to PML. A respective kit may be used to carry out a method according to the present invention. It may include one or more devices for accommodating the above components before, while carrying out a method of the invention, and thereafter.

As indicated above, any number of steps of a method according to the invention, including the entire method, may be performed in an automated way—also repeatedly, using for instance commercially available robots. As an illustrative example, the method may be an in vitro screening method, for example carried out in multiple-well microplates (e.g. conventional 48-, 96-, 384- or 1536 well plates) using automated work stations. The method may also be carried out using a kit of parts, for instance designed for performing the present method.

The listing or discussion of a previously published document in this specification should not necessarily be taken as an acknowledgement that the document is part of the state of the art or is common general knowledge.

The invention illustratively described herein may suitably be practiced in the absence of any element or elements, limitation or limitations, not specifically disclosed herein. Thus, for example, the terms “comprising”, “including,” containing”, etc. shall be read expansively and without limitation. Singular forms such as “a”, “an” or “the” include plural references unless the context clearly indicates otherwise. Unless otherwise indicated, the term “at least” preceding a series of elements is to be understood to refer to every element in the series. The terms “at least one” and “at least one of” include for example, one, two, three, four, or five or more elements. Slight variations above and below the stated ranges can be used to achieve substantially the same results as values within the ranges. Also, unless indicated otherwise, the disclosure of the ranges is intended as a continuous range including every value between the minimum and maximum values. In a further aspect, the invention also relates to the use of a kit comprising CD62L and optionally LFA-1 binding assay to determine the immune competence of a patient undergoing a treatment comprising VLA-4 blocking agent. The binding assay preferably comprises a CD62L and optionally a LFA-1 capture probe as described earlier. The kit enables the assessment of the risk for PML during the course of the treatment. Put it differently, CD62L can be seen as dynamic biomarkers which can assist the physician to determine whether to stop, continue, or resume the treatment of VLA-4 blocking agent, or to make any other suitable adjustment of the treatment regimen.

The practice of the present invention will employ conventional techniques of molecular biology, microbiology, and immunology, which are within the skill of those working in the art. Such techniques are explained fully in the literature. Examples of particularly suitable texts for consultation include the following: Sambrook et al. (1989) Molecular Cloning; A Laboratory Manual (2d ed.); D. N Glover, ed. (1985) DNA Cloning, Volumes I and II; M. J. Gait, ed. (1984) Oligonucleotide Synthesis; B. D. Hames & S J. Higgins, eds. (1984) Nucleic Acid Hybridization; B. D. Hames & S. J. Higgins, eds. (1984) Transcription and Translation; R. I. Freshney, ed. (1986) Animal Cell Culture; Immobilized Cells and Enzymes (IRL Press, 1986); Immunochemical Methods in Cell and Molecular Biology (Academic Press, London); Scopes (1987) Protein Purification: Principles and Practice (2d ed.; Springer Verlag, N.Y.); and D. M. Weir and C. C. Blackwell, eds. (1986) Handbook of Experimental Immunology, Volumes I-IV.

Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by exemplary embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the appending claims. In addition, where features or aspects of the invention are described in terms of Markush groups, those skilled in the art will recognize that the invention is also thereby described in terms of any individual member or subgroup of members of the Markush group.

In order that the invention may be readily understood and put into practical effect, particular embodiments will now be described by way of the following non-limiting examples.

### EXAMPLES

The examples illustrate techniques that can be used in methods according to the invention as well as exemplary embodiments of determining the level of T cells that express L-CD62L and/or LFA-1. Studies in recent years have come up with three statistical observations of predicting an MS patient's overall risk to later develop PML when treated with Natalizumab, but there is no possibility yet to measure an individual's PML risk. This, however, is urgently needed to facilitate the treatment decisions of clinicians and patients alike, as many patients opt for continuing treatment with Natalizumab even when their statistical risk to develop PML rises up to roughly 1:120 with all three risk factors present. As compromised immune surveillance has long been a hypothesis to explain the occurrence of an opportunistic infection, the inventors designed a study to include a variety of adhesion molecules on the surface of immune cell subpopulations. Patients analysed included inter alia MS patients under long-term therapy with Natalizumab, MS patients before escalation to Natalizumab, but with diverse prior immunemodulatory treatments such as e.g. Glatiramer Acetate, Interferon-β, Azathioprine, or Methotrexate.

The obtained data show that a surface molecule was strongly downregulated on T cells of patients who either would later develop PML. This molecule was CD62L, a member of the selectin family. In addition, LFA-1 was likewise strongly downregulated on T cells of MS patients under Natalizumab therapy before occurrence of PML. These markers are therefore risk predictors, because their expression pattern during Natalizumab therapy differed.

As the expression of L-Selectin was usually higher on CD4⁻ T cells (because in contrast to CD8⁺ T cells, there are no CD4⁺CD62L-CD45RA⁺ cells), it may in some cases be advantageous to use CD4⁺ T cells to determine risk when using L-Selectin.

Subjects Treated with Natalizumab

The status and longitudinal development, as well as function of major peripheral and CSF immune populations in patients under long-term treatment with Natalizumab were assessed. Focus of the following experiments underlying these examples was finding changes in the immune status of patients to possibly predict the occurrence of PML by assessing the impact of Natalizumab on T-cell function in combining immune phenotyping with functional in vitro and ex vivo assays.

Natalizumab, a humanized IgG4 antibody against the α-chain of VLA-4 (α4, CD49d), has been approved for the treatment of active relapsing-remitting Multiple Sclerosis (RRMS) since 2006. Long-term treatment with Natalizumab is associated with severe side effects, above all the development of progressive multifocal leukencephalopathy (PML). In addition to duration of treatment, previous immunosuppressive therapy (Panzara, M. A., et al. Multiple Sclerosis (2009) 15, 9, S132-S133) as well as the presence of JC virus, as ascertained by the presence of anti-JCV antibodies in serum, contribute to the risk of developing Natalizumab-associated PML (Bloomgren, G., et al. The New England Journal of Medicine (2012) 366, 20, 1870-1880). When all these risk factors are present, the statistical risk of PML can be as high as 1:120 (Clifford, D. B., et al. Lancet Neurology (2010) 9, 4, 438-446; Bloomgren et al., 2012, supra). While it is still unclear why and how (long-term) treatment with anti-CD49d contributes to the development of PML (Tan and Koralnik, 2010, supra), a multifactorial scenario is likely, including impaired immune surveillance and active JC virus replication (Schwab, N., et al. Multiple sclerosis [Houndmills, Basingstoke, England] (2012) 18, 3, 335-344; Schwab, N., et al. Neurology (2012) 78, 7, 458-467).

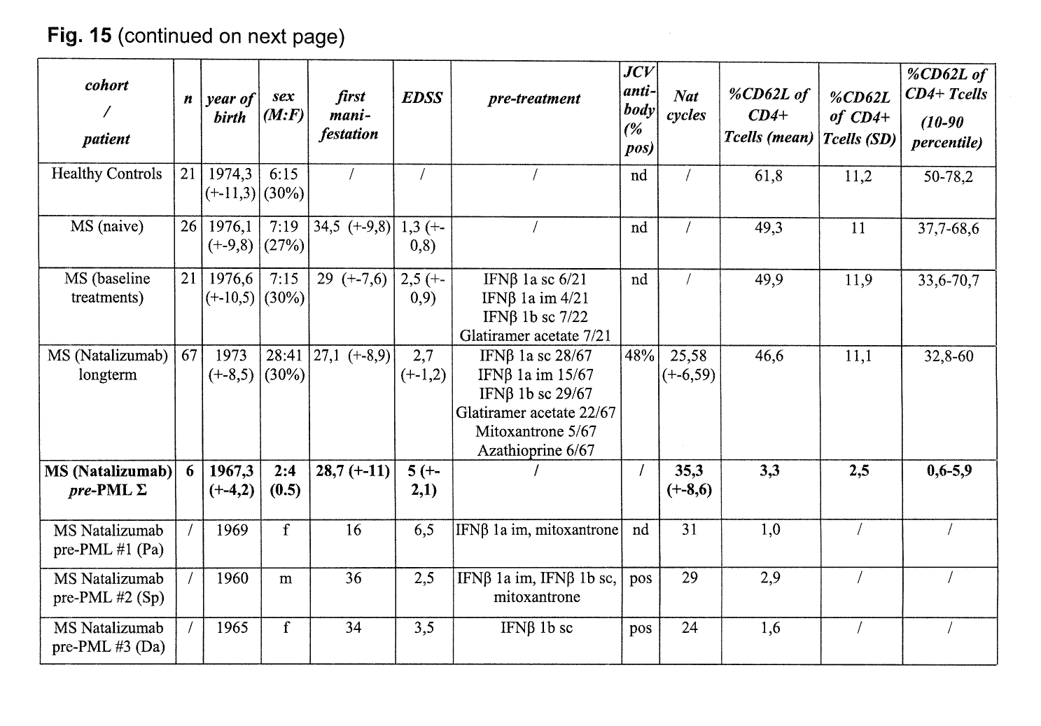

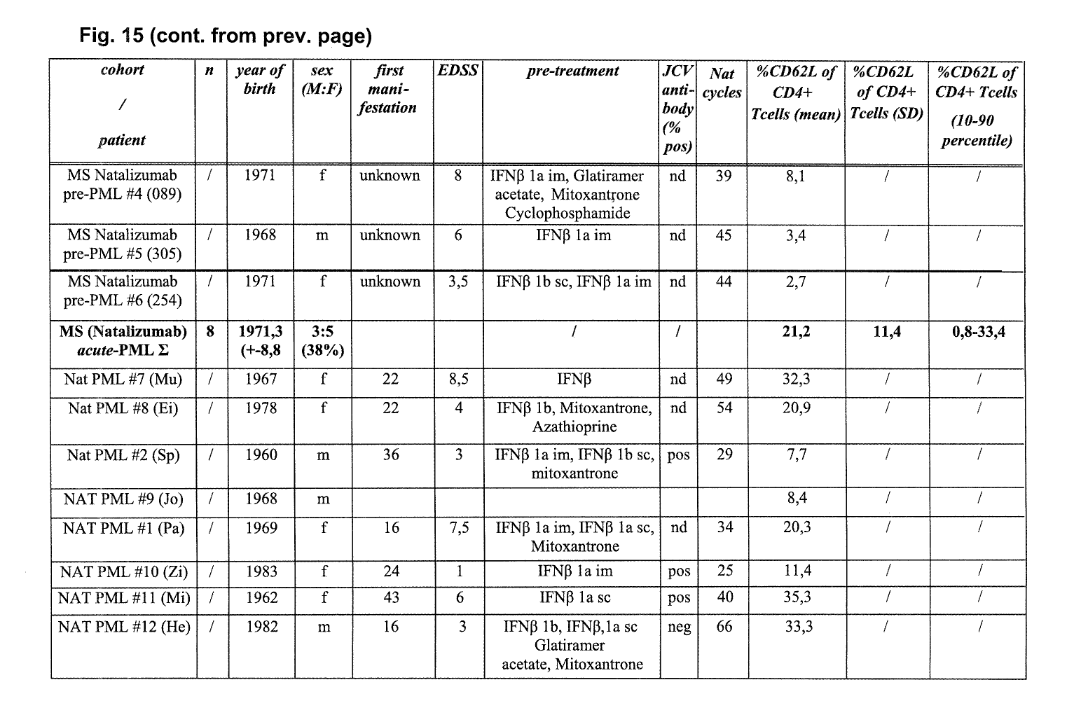

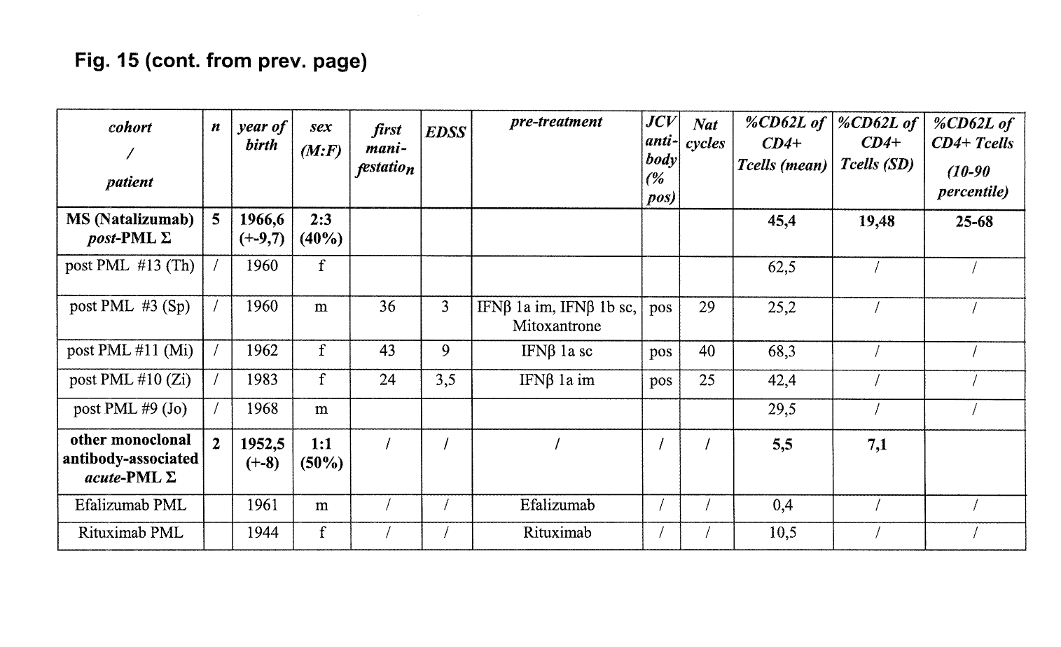

114 patients with the diagnosis of clinically definite active RRMS according to the 2005 revised McDonald diagnostic criteria (Polman, C. H. et al., Ann Neurol (2005) 58, 840-846) were enrolled in this study. 67 MS patients had continuously been treated with Natalizumab for 18-66 months, 21 MS patients received baseline immune-modulatory treatments (Interferons, Glatiramer acetate) and 26 MS patients were untreated and clinically as well as MRI-stable (cf. also Table 1, supra, for 22 of these patients). Age and sex-matched healthy donors (HD) with no previous history of any neurological or immune-mediated diseases served as controls. Furthermore, samples were available from different therapy-associated PML conditions: Natalizumab-associated (n=13), Rituximab-associated (n=1) and Efalizumab-associated (n=1). Six cases of HIV-associated PML served as additional controls. In 6 of the 13 Natalizumab-associated PML cases pre-PML samples were available (FIG. 15). The Table depicts average+standard deviation (if applicable).

The study was approved by the local ethics committee (Ethik-Kommission der medizinischen Fakultät der Universität Würzburg, registration number 155/06; Ethik-Kommission der Ärztekammer Westfalen-Lippe and der Medizinischen Fakultät der Wesfälischen Wilhelms-Universität, registration number: 2010-245-f-S) and informed written consent was obtained from all participants. This study was performed according to the Declaration of Helsinki.

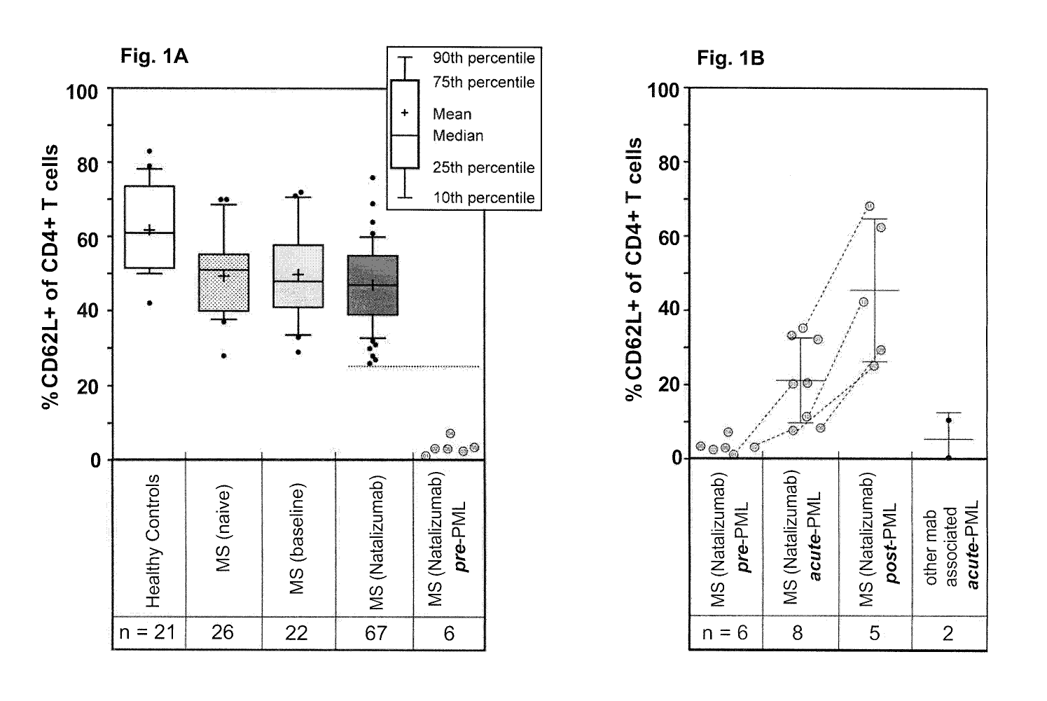

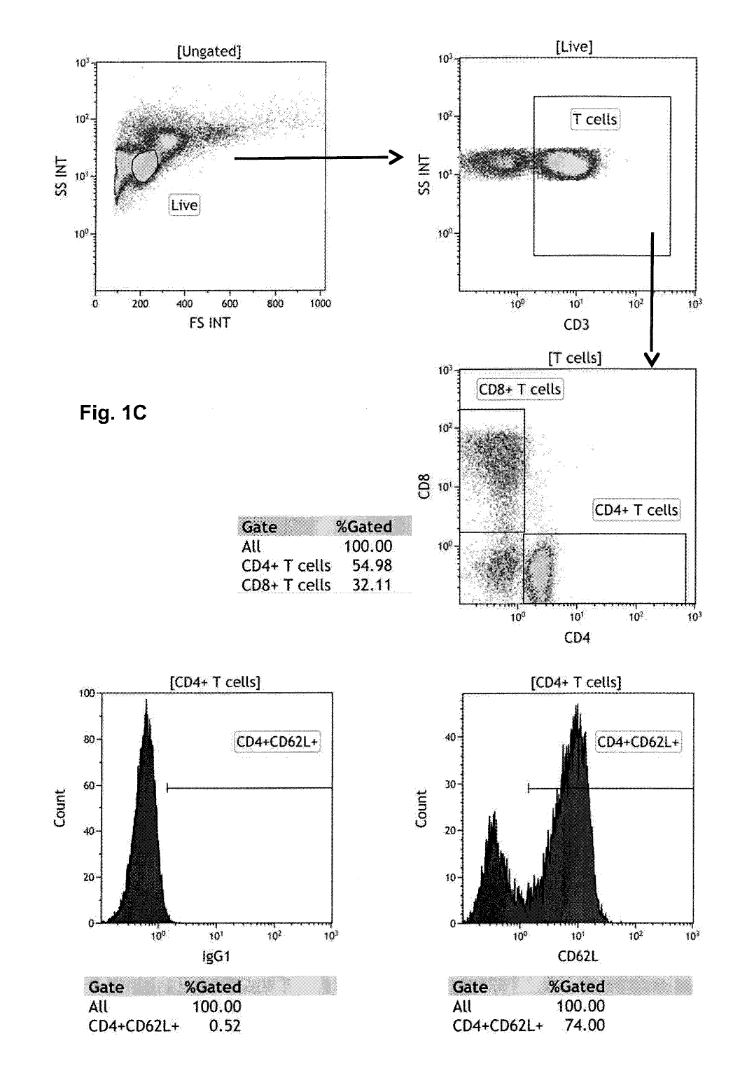

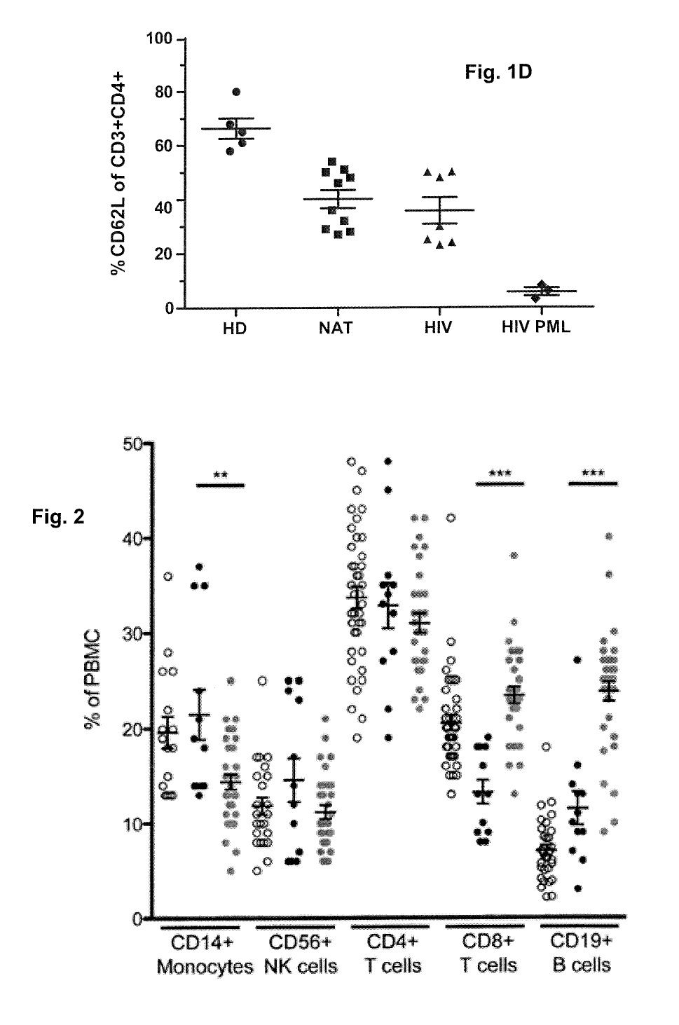

Data shown in FIG. 1D, FIG. 7, FIG. 8 and FIG. 9 are based on a smaller group of patients. 52 patients with the diagnosis of clinically definite active RRMS according to the 2005 revised McDonald diagnostic criteria were enrolled. Analyzed MS patients had been treated continuously with Natalizumab for 18-50 months and were stable by assessment of clinical and MRI parameters. 18 patients among this cohort underwent analysis of CSF in parallel to assessment of peripheral blood. 39 patients were followed longitudinally from treatment initiation. Two patients developed PML after 26 or 29 months, respectively. 45 age and sex-matched healthy donors (HD) with no previous history of neurological or immune mediated diseases served as controls. Furthermore, 22 untreated MS patients served as controls (Table 1, supra). PBMC from patients suffering from PML (HIV⁺ (n=4), Natalizumab-associated (n=3), Rituximab-associated (n=1), Efalizumab-associated (n=1)) were used as additional controls.

Peripheral blood (n=52) and cerebrospinal fluid (n=18) from patients under Natalizumab therapy (≧18 months) were analyzed using flow cytometry and in vitro transendothelial migration assays.

Data shown in FIG. 3 are based on a group of patients of yet different size. 78 patients with the diagnosis of clinically definite active RRMS were included. Analyzed MS patients had been treated continuously with Natalizumab for 18 to 60 months and were stable by assessment of clinical and MRI parameters. Five patients developed PML. In addition, samples were obtained from 30 patients with the diagnosis of clinically definite active RRMS before treatment with Natalizumab. 73 age and sex-matched healthy donors with no previous history of neurological or immune mediated diseases served as controls.

### Isolation of PBMC and Flow Cytometric Analysis

Peripheral blood mononuclear cells (PBMC) freshly isolated from EDTA blood were isolated by density gradient centrifugation using lymphocyte separation medium (PAA Laboratories, Pasching, Austria) as described previously in Schwab et al J Immunol. (2010) 184, 9, 5368-5374, incorporated herein by reference in its entirety. Flow cytometry analysis of CSF was performed as described in Schwab et al. Multiple Sclerosis (2009) 15, S275-S275, incorporated herein by reference in its entirety. Cells were then typically cryopreserved in freezing medium (50% RPMI 40% FCS 10% DMSO).

Ex vivo isolated, cultured or thawed cells were washed with FACS®-buffer (phosphatebuffered saline (PBS) supplemented with 0.1% bovine serum albumine (BSA) and 0.1% NaN₃) or staining buffer (phosphate-buffered saline (PBS) supplemented with 0.1% bovine serum albumine (BSA) and 200 mM EDTA). Cells were subsequently stained with fluorescence-labeled monoclonal antibodies (Mab) together with blocking mouse IgG (Sigma-Aldrich, Hamburg, Germany) at 4° C. for 30 min or at room temperature for 15 min. After washing once with staining buffer, cells were immediately measured on a FACSCalibur (BD Biosciences, Heidelberg, Germany) and Gallios™ Flow Cytometer (Beckman Coulter, Krefeld, Germany) and analyzed using FlowJo (Tree Star, Ashland, Oreg., USA) and Kaluza (Beckman Coulter) software. It should be noted that the presence of CD62L on the cell surface tends to be unstable, so that the staining buffer cannot contain sodium azide and measurement needs to take place immediately after the staining procedure.

In particular, LFA-1 protein was stained for CD11a, the α-chain of LFA-1. VLA-4 was stained for CD49d (the α-chain of VLA-4), as CD49d is the precise molecule blocked by Natalizumab.

Monoclonal immunoglobulins used in these examples were anti-CD62L (DREG-56, BioLegend), anti-CD3 (UCHT1, Beckman Coulter), anti-CD4 (13B8.2, Beckman Coulter), anti-CD8 (B9.11, Beckman Coulter), anti-CD11a (HI111, BD Pharmingen), anti-CD14, (MoP9, BD Biosciences), anti-CD19 (HIB19, BD Biosciences), anti-CD45 (J33, Beckman Coulter), anti-CD45RA (HI100, Beckman Coulter), anti-CD56 (NCAM 16.2, BD Biosciences), anti-CD49d (9F10, Biolegend) and anti-CD197 (3D12, BD Biosciences).

### Immunohistochemistry

Retrospectively investigated were 2 chordoid plexus tissue samples (autopsies) from 2 multiple sclerosis patients (both female, 31 and 72 years), and 15 tissue samples from patients without neurological diseases (11 men, 4 women; between 34 and 81 years, mean 60 years). The study was approved by the Ethics Committee of the University of Muenster. For histological analysis the paraffin embedded tissue samples were cut in 4 μm thick sections and stained with haematoxylin and eosin (HE). Immunohistochemistry for mouse anti-human CD3 (1:25) (Dako, Denmark) was performed using an automated immunostainer (autostainer Link48, Dako) and the avidin-biotin technique. Steamer pretreatment (citrate buffer pH6.1) for better antigen retrieval was performed.

### In Vitro Migration Assays

Primary human brain microvascular endothelial cells (HBMEC) and primary human choroid plexus epithelial cells (HCPEpiC) were purchased from ScienCell Research Laboratories (San Diego, Calif., USA). Cells were cultured on filter membrane of Transwells (3 μm pore size; Corning, N.Y., USA) for three days until reaching confluence.

Transmigration assays were performed essentially as described in Schneider-Hohendorf et al. Eur J Immunol. (2010) 40, 12, 3581-3590, incorporated herein by reference in its entirety. Briefly, PBMC in 100 μl of pre-warmed RPMI medium (RPMI, Penicilline/Streptamycine (1%), B27 supplement (2%) [Invitrogen, Darmstadt, Germany]) were added to the top of the HBMEC monolayers, and 600 μl of medium were added to the outer chamber of the inserts. The cells were allowed to migrate for six hours in a humidified cell culture incubator at 37° C. and 5% CO₂. Absolute counts of T cells were measured with Flow-Count Fluorospheres following the manufacturer's instructions (Beckman Coulter) to normalize the migration rates to standardized bead concentrations.

### Statistical Analysis

Statistical significance of differences between two groups was determined using unpaired Student's t-test except for comparisons between peripheral blood and CSF of the same patient, where the paired Student's t-test was used. Differences were considered statistically significant with p* values<0.05, with p**<0.01 and p***<0.001. Software for statistical and correlation assessment was Prism 5 (GraphPad, La Jolla, Calif., USA).

### Changes in the Composition of Major Immune Subsets Under Long-Term Natalizumab Therapy

Characterization of the major peripheral immune cell subpopulations in patients under long-term treatment with Natalizumab (n=34, treatment≧18 months) (FIG. 2). The percentage of CD4⁺ T cells did not deviate significantly from healthy donors and untreated MS patients. The CSF compartment of these patients (n=18) was characterized by reduced percentages of B cells and CD4⁺ T cells compared to peripheral blood. The CD4/CD8 ratio in the CSF was reduced to 0.54 (11.8:21.8), indicating a stronger effect of Natalizumab on CD4 than CD8 T cells (FIG. 4).

### Impact of Long-Term Natalizumab Treatment on T-Cell Function and Phenotype

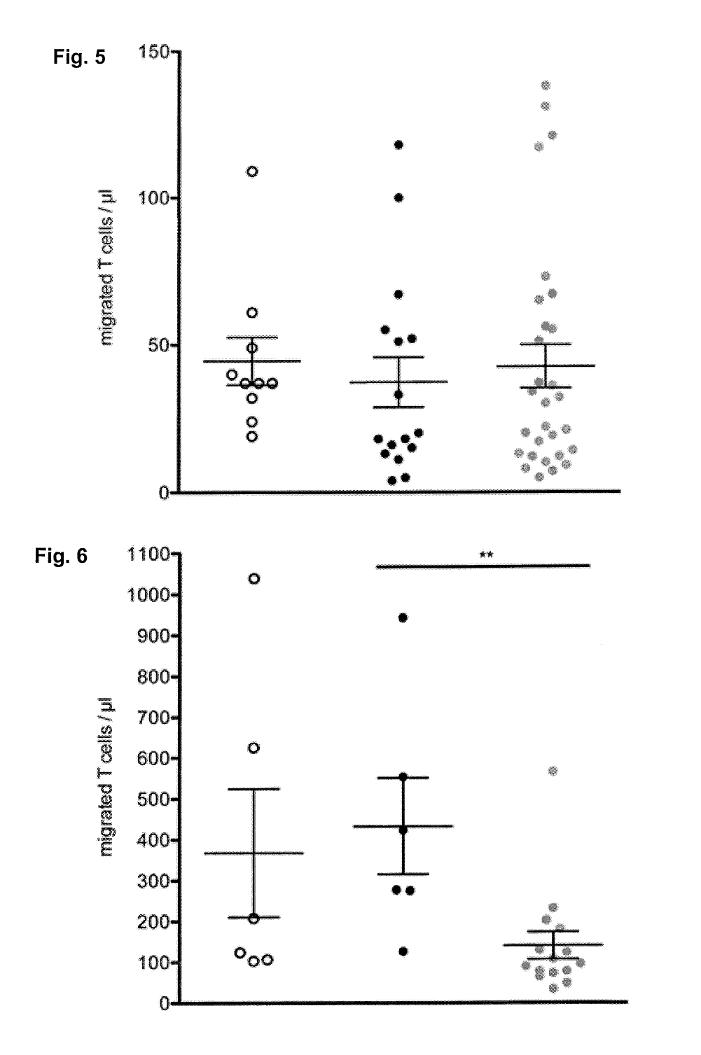

As published previously (Defer, G., et al., J Neurol Sci (2012) 314, 1-2, 138-142; Harrer, A., et al., J Neuroimmunol (2011) 234, 1-2, 148-154), treatment with Natalizumab influenced the expression of CD49d on patients' peripheral CD4⁻ T cells over time. However, it could be observed that after a decrease of surface expression to a minimum at month six of treatment, the CD49d levels surprisingly recovered. Of note, it could be shown that a patient, who developed antibodies against Natalizumab, did not downregulate CD49d on CD4⁺ T cells, which might easily be used as an early marker for the detection of patients who will not benefit from Natalizumab due to production of antibodies, as previously suggested by (Defer et al., 2012, supra). Never-the-less, CSF flow cytometry showed that CD49d levels on CD4⁺ T cells were undetectable in these patients compared to their peripheral counterparts, independent of the peripheral recovery (FIG. 2B), whereas it has been shown repeatedly that control MS patients usually show a strongly enhanced CD49d expression on CSF T cells when compared to the periphery (data not shown and (Barrau, M. A. et al., J Neuroimmunol (2000) 111, 215-223). Additionally, CSF CD4⁺ T cells in patients under long-term treatment were characterized by missing expression of CD45RA and CCR7 (indicating an effector memory phenotype). This stands in contrast to the central-memory-like phenotype (CD45RA-CCR7⁺), which has been published previously for MS patients (Kivisäkk, P., et al., Ann Neurol (2004) 55, 627-638). Similar results were obtained for CD8⁺ T cells (data not shown). Effector memorycompartments (as determined by CCR7 expression) in the periphery were not significantly affected by Natalizumab long-term therapy (data not shown)(Planas, R., et al., Eur J Immunol. (2011) doi: 10.1002/eji.201142108). CSF is generated in the choroid plexus (CP), which has also been shown in animal models to be the main entry site for leukocytes during CNS immune surveillance (Carrithers, M. D., et al., Brain (2000) 123 (Pt 6), 1092-1101) as well as inflammation (Reboldi, A., et al., Nat Immunol (2009) 10, 514-523). The inventors could show that this route is a possible entry site for T cells in the human system during homeostatic as well as pathological conditions. In both MS tissue samples as well as in 7 out of 15 controls we detected CD3 positive cells in the choroid plexus. The majority of T cells was located perivascularly, however we observed also single T cells in close proximity to the epithelium. As administration of Natalizumab is assumed to reduce CNS-invasion of leukocytes by inhibiting immune cell adhesion to endothelial cells of the blood-brain barrier (BBB), it was unexpected that quantitative comparison of individual migration through primary human brain-derived microvascular endothelium revealed a strong heterogeneity among Natalizumab-treated patients compared to healthy controls or untreated MS patients (FIG. 5), even though all treated patients were considered clinically stable. In contrast to this, diapedesis through primary choroid plexus-derived epithelium (simulating the blood-CSF barrier) revealed a significant and homogeneous reduction in long-term Natalizumab-treated patients (FIG. 6). As the inventors had observed a relation between the expression of CD49d and treatment duration, they decided to analyze the apparent heterogeneity of transendothelial migration in relation to the months of Natalizumab treatment in more detail.

### Longitudinal Assessment of T-Cell Function Under Natalizumab Treatment: Implications for the Development of PML

Therapy-associated PML has developed as a significant challenge in a number of medical specialties over the past several years (Vinhas de Souza, M., et al., Clinical Pharmacology and Therapeutics (2012) 91, 4, 747-750). Natalizumab-associated PML has attracted considerable attention, since anti-CD49d treatment has been associated with a particularly large number of PML cases in a population, which is traditionally not at risk. Three factors have been identified that can be used as risk stratification tools. Two, namely prior immunosuppressant use and duration of therapy, are based on statistical observations, while one, presence of anti-JCV antibodies, is based on a patient's specific biologic parameter. However, even JCV seropositivity is relatively non-specific, since it simply identifies patients who have had or currently have a JCV infection and therefore the theoretical possibility of developing PML, which is JCV-mediated (Panzara et al., 2009, supra; Clifford et al., 2010, supra; Gorelik, L., et al. Annals of Neurology (2010) 68, 3, 295-303; Bloomgren et al., 2012, supra). A method to measure an individual's biological response to treatment as a way to monitor for PML risk is urgently needed. We used the following groups of blood donors to differentiate between effects of MS, pre-treatments, and Natalizumab: 1) healthy controls, 2) treatment-naive MS patients, 3) MS patients before treatment with Natalizumab and 4) MS patients under long-term therapy with Natalizumab (18-66 months). The Natalizumab-treated subjects were recruited from five separate cohorts (Würzburg, Münster, Osnabrück (Germany), French Cohort Study (France) and Brascia (Italy)). In part among these five cohorts, we had access to samples from 13 PML patients. Importantly, six of these patients had given blood before the diagnosis of PML (19, 26, 4, 15, 21, 20 months before PML diagnosis). As additional controls, we analyzed samples from non-Natalizumab patients who developed PML (both therapy-associated and HIV-associated; see study design and Table 1). Surprisingly, our results showed that the percentage of CD62L expressing cells was consitently much lower (by more than tenfold) on CD4 T cells of patients who would later on develop PML with a mean of 3.3% compared with a mean of 46.6% from non-PML Natalizumab patients (FIG. 1A and FIG. 15 for individual dot plots).

Furthermore, samples from patients suffering from acute PML also showed a reduction or lack of CD62L expression, indicating a persistent dysregulation at least up to the point of PML diagnosis. CD62L expression showed a more diverse pattern in PML patients post diagnosis, perhaps due to the acute treatments administered for management of the PML. After PML (recovery phase, post immune reconstitution syndrome, IRIS), the percentage of CD4⁺ cells expressing CD62L returned to a more normal range (45.4%) (FIG. 1B). Surface expression of CD62L on CD4⁺ T cells was higher than on CD8⁻ T cells. Therefore, the detection of CD62L levels on CD4⁺ T cells allowed for the most accurate discrimination of patients who eventually developed PML (data not shown). Of note, the present inventors found that using the percentage of positive cells against the isotype (in contrast to the MFI) gave the most reproducible results on different flow cytometers. The detailed gating is sketched in FIG. 15.

Expression levels of CD62L and LFA-1 were followed longitudinally in 39 Natalizumab patients in relation to transendothelial migration. Notably, levels of peripheral LFA-1 (FIG. 7) and CD62L (FIG. 8) showed a pronounced decrease within the first months, with a minimum at 6 months of therapy, followed by a subsequent gradual recovery. Functionally, this shift (reduced levels of CD49d, LFA-1, and CD62L) lead to a pronounced reduction of T-cell migration until 6 months of therapy and a subsequent recovery (FIG. 9). Between months 3 and 12 of treatment, transendothelial migration of T cells in vitro is severely reduced, which coincided with the reduced expression of CD62L and LFA-1.

Two patients in the cohort on which the data of FIG. 1D, FIG. 7 and FIG. 8 are based developed PML after 26 (FIG. 9, grey circles) and 29 (FIG. 9, white circles) months of therapy. Analysis of these patients' samples (time point 0 was not available) revealed that, in contrast to the normal development, levels of LFA-1 on CD4⁺ T cells further decreased after 12 months of therapy instead of the expected recovery (FIG. 7). Additionally, CD62L expression was completely absent during the investigated time frame for one of the patient who later developed PML (FIG. 8) and the migration of T cells was already very low at month 1. Migratory function did not recover over time (FIG. 9). Notably, analysis of one of these patients more than one year after PML revealed a restored transendothelial migration/CD11a expression with poor recovery of CD62L expression.

Compared to the control patients (patients who did not develop PML), PML patients showed a lack of LFA-1 recovery, (FIG. 7), a lack or reduced of CD62L expression and a lack of CD62L recovery (FIG. 8), and reduced transendothelial migration (FIG. 9).

Patients suffering from PML (n=8) associated with HIV infection or treatment with monoclonal antibodies (Natalizumab, Rituximab, Efalizumab) showed a similar lack of CD62L expression on the surface of CD4⁺ T cells at the beginning of their PML. This was again not associated with a shift towards effector memory T cells as delineated by CD45RA/CCR7 stainings (data not shown).

Perhaps importantly, the effector-memory distribution (assessed by CCR7) (Schwab et al., Multiple Sclerosis, 2012, supra; Sottini, A., et al. PLoS ONE (2012) 7, 4, e34493) of two of these patients was also altered, whereas the other four were comparable to HDs (data not shown). This may define a group with inherent risk of PML development under specific conditions.

While more research is needed, the inventors' results suggest a possible treatment paradigm where, after more than 18 Natalizumab infusions (months of therapy), the percentage of CD62L positive CD4⁺ cells is assessed. If the CD62L level drops below a defined threshold, which in this study could be set to approximately 25%, (FIG. 1A, dotted line: defined as two times the standard deviation (SD) from the mean (m) of the control cohort (mean=46.6; SD=11.1; threshold=24.5)) an early re-assessment (e.g. one month later) of the percentage of CD62L expressing T cells may be advisable. Continuous lack of CD62L could indicate a higher risk of PML and warrant either very close clinical monitoring or a potential change in treatment regimens (Natalizumab cessation). As acute PML appears to exert variable, but not well understood effects on the immune system, CD62L should not be used as a method of PML diagnosis per se, but rather as a prospective risk factor for developing PML in the future.

Taken together, the present cell-based assay for PML risk prediction may provide an immensely valuable tool for patients and practitioners in the field of MS treatment, albeit it needs to be further validated in larger, multicenter cohorts, as well as using more patient samples collected before development of PML.

### Real Time PCR Analysis

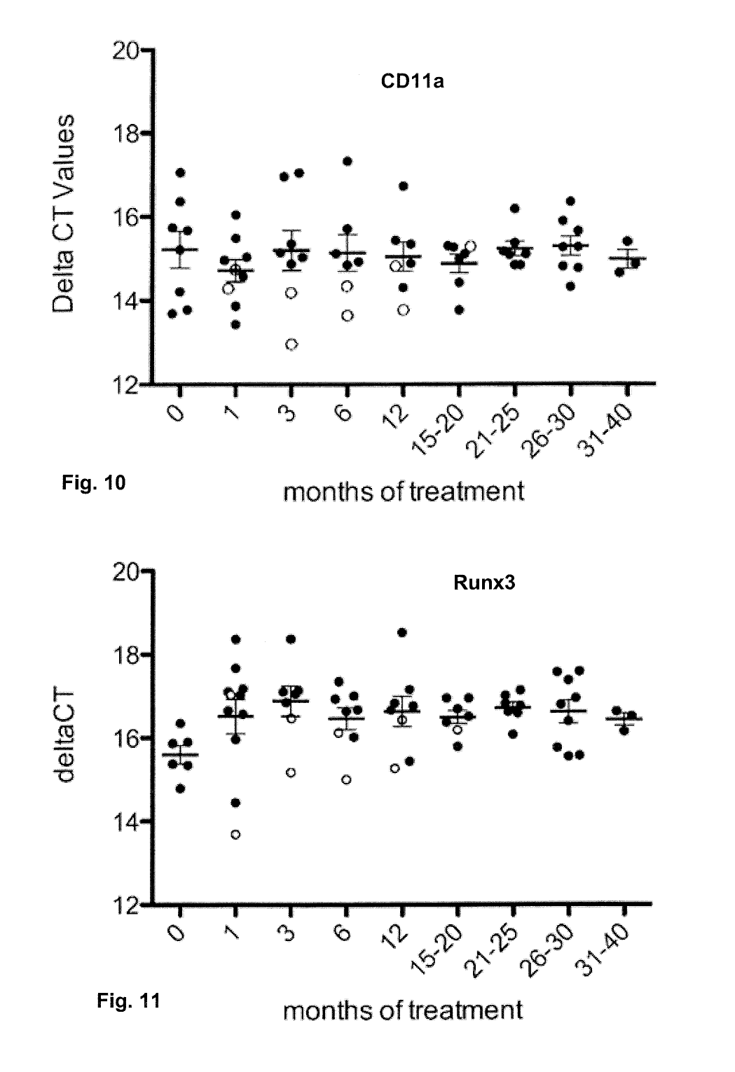

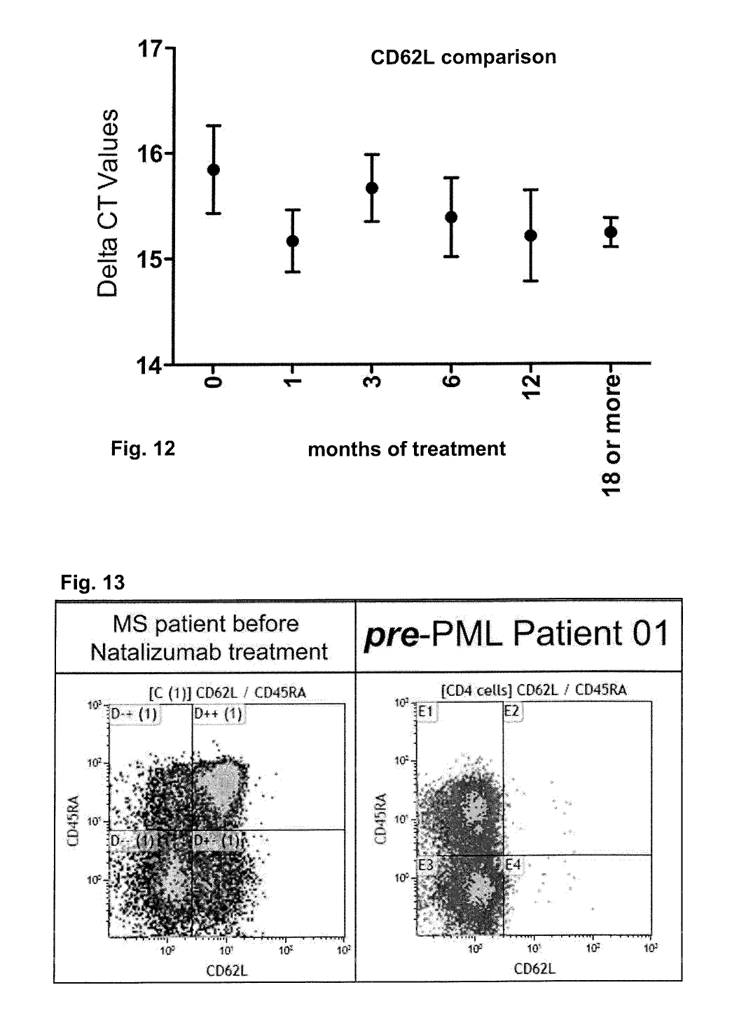

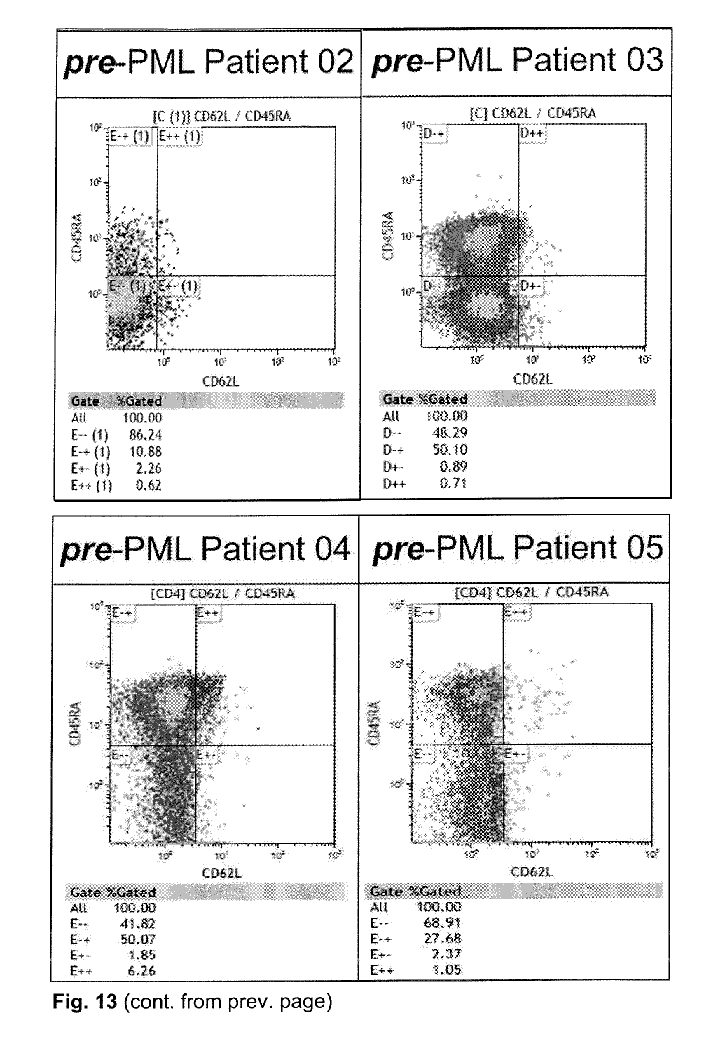

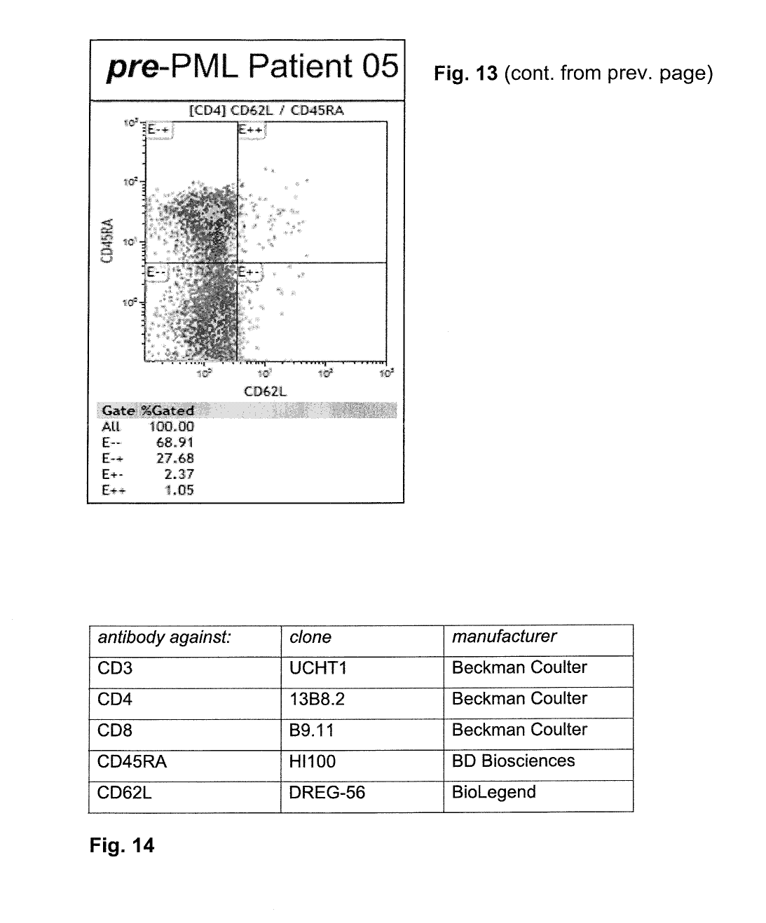

RNA isolation was performed using Trizol® (Invitrogen, Karlsruhe, Germany) following the manufacturer's instructions. mRNA was transcribed using random hexamers and MuLV reverse transcriptase (all reagents supplied by Applied Biosystems, Foster City, USA). Gene expression assays for the detection and quantification of CD11a, Runx3 and CD62L and the housekeeping gene hS 18 were purchased from Applied Biosystems and used according to the manufacturer's protocol. The Applied Biosystems Step-One Plus real-time PCR system was used, all samples were run in duplicates and each run contained several controls (healthy donor samples, wells without cDNA). There were no significant differences in cycle threshold neither within nor between the experiments. Quantification of gene expression was performed by comparing the amplification efficiencies of targets and housekeeping gene. All samples were normalized to hS18. Therefore, a lower CT value equals a higher expression of mRNA of the specific target. FIGS. 10-12 show the relative quantification of CD11a, Runx3, and CD62L as compared to hS18 on thawed PBMC from MS patients before (month 0) and in the time course of therapy (months 1, 3, 6, 12, 15-20, 21-25, 26-30, 31-40, 41-50; n=27 patients) as assessed by real-time PCR.

Long-term treatment with Natalizumab leads to changes in the peripheral immune subset distribution, which is in accordance to previous reports (increased numbers of peripheral B cells, attributed to the recruitment of precursor B cells (Krumbholz, M., et al., Neurology (2008) 71, 1350-1354) and decreased numbers of peripheral CD14⁺ monocytes (Skarica, M., et al., J Neuroimmunol (2011) 235, 1-2, 70-76). The increase in peripheral CD8⁺ T cells with no significant changes in the CD4 compartment might possibly contribute to the reversed CD4/CD8 ratio in the CSF, as observed in the cohort of these Examples and previously published (Stüve, O, et al., Arch Neurol (2006) 63, 1383-1387). Not mutually exclusive, CD4 cells might also undergo apoptosis upon encountering the antibody for a prolonged period of time, which has been published for short-term exposure in vitro (Kivisäkk, P., et al., Neurology (2009) 72, 1922-1930). The alterations in CD62L and LFA-1 expression on T cells, which have previously been shown for their CD34⁺ stem cells (Jing, D., et al., Bone Marrow Transplantation (2010) 45, 1489-1496), might also be due to the co-localization of CD49d with CD62L on cell surface microvilli (Wedepohl, S., et al., Eur J Cell Biol. (2012) 91, 4, 257-264). In contrast to CD62L, LFA-1 is solely expressed on the planar cell body (ibid.), suggesting that the expression of LFA-1 is regulated on the gene expression level, as the connection between CD49d and LFA-1 has been shown in the inverted setting, where the blockade of CD11a increased the percentage of CD49d⁺ T cells (Harper, E. G., et al., J Invest Dermatol (2008) 128, 1173-1181).

LFA-1 and CD62L have previously been used together with CD45RA as markers to distinguish naïve, central-memory, and effector-memory T cells (Maldonado, A., et al., Arthritis Res Ther (2003) 5, R91-R96; Okumura, M., et al., J Immunol (1993) 150, 429-437). These subpopulations differ in their functional tasks with central-memory cells conferring immunity against viruses and cancer cells and effector-memory cells producing cytokines like IFN-γ and IL-4 (reviewed in (Wherry, E., et al., Nat Immunol. (2003) 4, 3, 225-234). The CSF of MS patients has been shown to mainly consist of central memory cells (Giunti, D., et al., J Leukoc Biol (2003) 73, 584-590; Kivisäkk et al., 2004, supra). This population is known to be involved in immune-mediated CNS damage during EAE (Grewal, I.S., et al., Immunity (2001) 14, 291-302) invading the CNS via the choroid plexus (Reboldi, A., et al., Nat Immunol (2009) 10, 514-523). The CSF of patients under long-term treatment with Natalizumab, however, almost exclusively contains effector-memory-like T cells. Furthermore, transepithelial migration of long-term treated Natalizumab patients is permanently reduced while transendothelial migration recovers during long-term therapy. The choroid plexus divides blood and CSF consisting of two barriers, one endothelial barrier on the blood side and one epithelial on the CSF side (Engelhardt, B., et al., Microsc Res Tech (2001) 52, 112-129) and reviewed by (Wilson, E. H., et al., J Clin Invest (2010) 120, 1368-1379). In line with previous findings in the murine system, showing that CD49d is mandatory for adhesion to the epithelial-, but not to the endothelial barrier (Steffen, B. J., et al., Am J Pathol (1996) 148, 6, 1819-1838) of the choroid plexus, it is conceivable that Natalizumab efficiently impairs this route to the CSF, resulting in a low cell count in the CSF of patients and the clinical anti-inflammatory effects. Immune surveillance, which can be accomplished using alternative routes e.g. via the subarachnoid space or directly through the blood brain barrier (reviewed by Hickey, W. F., Semin Immunol (1999) 11, 125-137), should still be functional in patients under long-term treatment with Natalizumab, as they only require crossing an endothelial barrier. In line with this, the T cells in the CSF of Natalizumab patients do not express CD49d, indicating that these cells did not use the choroid plexus as entry site into the CNS. It was shown very recently that Th17 cells in EAE migrate into the spinal cord independently of a4 integrin, whereas Th1 cells, which are supposed to be mainly responsiblefor MS pathology, use a4 integrin for the migration into the brain (Rothhammer, V., et al., J Exp Med. (2011) 21, 208, 12, 2465-2476). The invasion of these putatively pathogenic Th1 cells would therefore be inhibited by Natalizumab.

Between months 3 and 12 of treatment, transendothelial migration of T cells in vitro is severely reduced. This coincides with a reduced expression of LFA-1 and CD62L, both being molecules imperative for endothelial migration (reviewed by (Ransohoff et al., Nat Rev Immunol. (2003) 3, 7, 569-581). Interestingly, this fits to previously published data, showing peaking JCV-, but also Epstein-Bar-, Cytomegalo- and MOBP-specific T-cell responses in the same time frame indicating that the majority of primed effector T cells are efficiently trapped in the periphery (Jilek, S., et al., Lancet Neurol. (2010) 9, 3, 264-272). As a side note, the observed modulation of LFA-1 should have major implications for T-cell function besides migration, such as formation of the immunological synapse together with CD49d, cytotoxicity and antigen-specific restimulation (Mittelbrunn, M., et al., Proc Natl Acad Sci U.S.A. (2004) 27, 101(30):11058-63; Rutigliano, J. A., et al., 2004, J Virol. (2004) 78, 6, 3014-3023; Yarovinsky, T. O., et al., Am J Respir Cell Mol Biol. (2003) 28, 5, 607-615). Admittedly, the applied migration paradigms can only partly reflect the in vivo situation, as especially the inflammatory milieu at stages of a possible MS relapse cannot be simulated properly in vitro to date. Nevertheless, a non-inflamed cellular barrier lacking attracting stimuli on the basolateral side most likely reflects the conditions of basic immune surveillance which we consider as more important in terms of controlling a JCV reactivation event. Furthermore, the in vitro paradigms were designed to identify individuals at risk of PML on a large scale and therefore were kept as basic as possible to enable maximum experimental reproducibility.

Five patients in the inventors' cohort developed PML. One of these patients has previously been described in a case report, mainly focusing on the immune response during PML and subsequent IRIS (Schwab, N., et al., Mult Scler. (2012) 18, 335-344). Strikingly, all 5 PML patients shared three remarkable differences to the rest of the investigated cohort: 1) reduced transendothelial migration over the complete time frame, 2) missing LFA-1 recovery after 12 months, 3) missing CD62L expression and recovery. Data from Natalizumab-associated PML patients after plasma exchange revealed that migration rates normalized after stopping the Natalizumab treatment, while CD62L expression only recovered to some extent. This might hint towards a possible pre-existing condition in some patients, possibly associated with a predisposed shift in effector/memory T-cell compartments (Schwab et al., 2012, supra). All patient samples at the beginning of the PML showed the very characteristic absence of CD62L while leaving the effector-memory percentages intact (assessed by CCR7). It should be noted that especially naive (CD45RA⁻CCR7⁻) CD4 T cells lacking the expression of CD62L do not exist in controls. CD62L might therefore be the first dynamic biomarker linking all different types of PML (antibody-associated concerning treatment with Natalizumab, Efalizumab, and Rituximab, as well as HIV-associated). Further studies need to be conducted to find out if the loss of CD62L contributes to the development of PML or whether it is not functionally associated, but rather symptomatic.

Taken together, the above data support the assumption that part of the clinical efficacy of Natalizumab is due to a selective inhibition of the T-cell trafficking route through the choroid plexus into the CNS responsible for the entry of effector cells during inflammatory events i.e. MS relapse. Absent recovery of transendothelial migration could result in impaired basic CNS immune surveillance, thereby increasing the risk for PML development. It cannot be excluded that other biomarkers might also be important in patients at enhanced risk for PML. Therefore, the inventors' hypothesis ought to be evaluated and expanded in larger cohorts. However, the inventors would suggest testing patients under long-term treatment for their capacity for transendothelial migration, their peripheral levels of LFA-1, and especially CD62L to assess basic immune competence. Patients showing compromised immune surveillance should be clinically monitored very closely.

1. A method of assessing the risk of occurrence of progressive multifocal leukoencephalopathy (PML) in a subject, the method comprising detecting the level of T cells expressing L-selectin (CD62L) in a sample from the subject, wherein a decreased level of CD62L expressing T cells, relative to a threshold value, indicates a risk of occurrence of PML.

2. The method of claim 1, wherein the subject is undergoing VLA-4 blocking agent treatment.

3. The method of claim 2, wherein the VLA-4 blocking agent is an immunoglobulin or a proteinaceous binding molecule with immunoglobulin-like functions.

4. The method of claim 1, further comprising detecting the level of T cells expressing lymphocyte function-associated antigen-1 (LFA-1) in the sample, wherein a decreased level of LFA-1 expressing T cells, relative to a threshold value, indicates a risk of occurrence of PML.

5. The method of claim 1, comprising detecting the level of T cells expressing CD62L , wherein a decreased level of CD62L, relative to the threshold value, indicates a risk of occurrence of PML.

6. The method of claim 1, wherein the method further comprises determining the migration of CD45⁺CD49⁺ immune cells.

7. The method according to claim 6, wherein the immune cells are T cells.

8. A method of stratifying a subject undergoing VLA-4 blocking agent treatment for suspension of the VLA-4 blocking agent treatment, the method comprising detecting the level of T cells expressing at least one of CD62L and LFA-1 in a sample from the subject, wherein a decreased level of CD62L and/or LFA-1 expressing T cells, relative to a threshold value, indicates that the subject is in need of a suspension of VLA-4 blocking agent treatment.

9. The method of claim 1, wherein the sample is one of a blood sample, a blood cell sample, a lymph sample and a sample of cerebrospinal fluid.

10. The method of claim 1, wherein the threshold value is based on the level of CD62L expressing T cells in a control sample

11. The method of claim 4, wherein the threshold value is based on the level of LFA-1 expressing T cells, as applicable, in a control sample.

12. The method of claim 1, wherein detecting the level of CD62L expressing T cells comprises detecting at least one of:
(i) the number of T cells in the sample from the subject that have CD62Lon the cell surface,
(ii) the amount of CD62L present on T cells of the sample from the subject, and
(iii) the amount of nucleic acid formation from the SELL gene encoding CD62Lin T cells of the sample from the subject.

13. The method of claim 4, wherein detecting the level of LFA-1 expressing T cells comprises detecting at least one of:
(i) the number of T cells in the sample from the subject that have LFA-1 on the cell surface,
(ii) the amount of LFA-1 present on T cells of the sample from the subject, and
(iii) the amount of nucleic acid formation from the ITGAL gene encoding CD11A and the ITGB2 gene encoding CD18 in T cells of the sample from the subject.

14. The method of claim 1, wherein the T cells are at least one of CD4+ T cells and CD8+ T cells.

15. The method of claim 1, comprising repeatedly detecting the level of at least one of CD62L expressing T cells and LFA-1 expressing T cells in a sample from the subject.

16. The method of claim 11, wherein (i) detecting the number of T cells in the sample that have CD62L on the cell surface and/or (ii) detecting the amount of CD62L present on T cells of the sample comprises contacting T cells in/of the sample with a capture probe, the capture probe being specific for CD62L, and detecting the amount of the capture probe binding to CD62L.

17. The method of claim 13, wherein (i) detecting the number of T cells in the sample that have LFA-1 on the cell surface and/or (ii) detecting the amount of LFA-1 present on T cells of the sample comprises contacting T cells in/of the sample with a capture probe, the capture probe being specific for LFA-1, and detecting the amount of the capture probe binding to LFA-1.

18. The method of claim 11, wherein detecting the number of T cells in the sample from the subject that have CD62L on the cell surface comprises determining the number of T cells in the sample that do not have CD62Lon the cell surface.

19. The method of claim 13, wherein detecting the number of T cells in the sample from the subject that have LFA-1 on the cell surface comprises determining the number of T cells in the sample that do not have LFA-1 on the cell surface.

20. The in vitro use of a capture probe specific for CD62L for at least one of (i) assessing the risk of occurrence of progressive multifocal leukoencephalopathy (PML) in a subject, and (ii) stratifying a subject undergoing VLA-4 blocking agent treatment for suspension of said VLA-4 blocking agent treatment.

21. The in vitro use of an antibody or a proteinaceous binding molecule with antibody-like functions specific for at least one of CD11A, CD18 and LFA-1 for assessing the risk of occurrence of PML in a subject or stratifying a subject undergoing VLA-4 blocking agent treatment for suspension of said VLA-4 blocking agent treatment.

22. A VLA-4 blocking agent for use in the treatment of a pathologic inflammatory disease within the CNS and/or an autoimmune disease so as to avoid the occurrence of PML, wherein the use comprises administration of the VLA-4 blocking agent to a subject over a period of time, followed by a discontinuation of the administration for a period of time.

23. The VLA-4 blocking agent of claim 22, wherein discontinuation of the administration of the VLA-4 blocking agent is effected after detection of a decreased or an increased level of CD62L and optionally LFA-1 expressing T cells in the subject, relative to a threshold value.


SEQUENCE LISTING (6 sequences)

SEQ ID NO: 1 - protein, 9 aa, Unknown
  Description of Unknown FLAG-peptide sequence
DYKDDDDKG

SEQ ID NO: 2 - protein, 10 aa, Unknown
  Description of Unknown T7 epitope sequence
ASMTGGQQMG

SEQ ID NO: 3 - protein, 11 aa, Herpes simplex virus
QPELAPEDPED

SEQ ID NO: 4 - protein, 11 aa, Vesicular stomatitis virus
YTDIEMNRLGK

SEQ ID NO: 5 - protein, 9 aa, Unknown
  Description of Unknown Hemagglutinin (HA) epitope sequence
YPYDVPDYA

SEQ ID NO: 6 - protein, 10 aa, Unknown
  Description of Unknown Myc epitope sequence
EQKLISEEDL